(19) United States
(12) Patent Application Publication
(10) Pub. No.: US 2017/0165259 A1
(43) Pub. Date: Jun. 15, 2017

(54) COMBINED MODULATION OF IRE1

(71) Applicants: THE REGENTS OF THE UNIVERSITY OF CALIFORNIA, Oakland, CA (US); UNIVERSITY OF WASHINGTON THROUGH ITS CENTER FOR COMMERCIALIZATION, Seattle, WA (US)

(72) Inventors: Dustin J. MALY, Seattle, WA (US); Bradley J. Backes, San Francisco, CA (US); Scott A. Oakes, San Francisco, CA (US); Feroz R. Papa, San Francisco, CA (US); Rajarshi Ghosh, Pacifica, CA (US); Likun Wang, San Francisco, CA (US)

(21) Appl. No.: 15/387,463

(22) Filed: Dec. 21, 2016

Related U.S. Application Data
(63) Continuation of application No. PCT/US2015/038906, filed on Jul. 1, 2015.
(60) Provisional application No. 62/019,827, filed on Jul. 1, 2014.

(51) Int. Cl. A61K 31/4985; A61K 45/06; A61K 31/381

(52) U.S. Cl. CPC A61K 31/4985; A61K 31/381; A61K 45/06

(57) ABSTRACT

Described herein, inter alia, are combined compositions of an Ire1 kinase modulating compound and an Ire1 ribonuclease modulating compound and methods of using the same.

### CROSS-REFERENCES TO RELATED APPLICATIONS

This application is a continuation of International Application No. PCT/US2015/038906, filed Jul. 1, 2015, which claims the benefit of U.S. Provisional Patent Application No. 62/019,827, filed Jul. 1, 2014, each of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant nos. OD001925, DK080955, GM086858, OD001926, awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 48536-554C01US_ST25.TXT, created on Dec. 15, 2016, 35,938 bytes, machine format IBM-PC, MS-Windows operating system, is hereby incorporated by reference.

### BACKGROUND

Cells often experience conditions during which the workload on the endoplasmic reticulum (“ER”) protein folding machinery exceeds its capability. Such cells are said to be experiencing “ER stress.” ER stress can result from secretory work overload, expression of folding-defective secretory proteins, deprivation of nutrients or oxygen, changes in luminal calcium concentration, and deviation from resting redox state. Sophisticated cellular surveillance and quality control systems work to maintain ER homeostasis under such perturbations. Under ER stress, secretory proteins accumulate in unfolded forms within the organelle to trigger a set of intracellular signaling pathways called the unfolded protein response (UPR). UPR signaling increases transcription of genes encoding chaperones, oxidoreductases, lipid-biosynthetic enzymes, and ER-associated degradation (ERAD) components (Travers, K. J. et al. Cell 101, 249-258 (2000)).

In some instances, the ER stressed state remains too great, and cannot be remedied through the UPR's homeostatic outputs. In these situations, the UPR switches strategies and actively triggers apoptosis (Zhang, K. & Kaufman, R. J. Neurology 66, S102-109 (2006)); we have named this destructive signaling state the Terminal UPR (signature events of the Terminal UPR are described herein). Apoptosis of irremediably stressed cells is an extreme, yet definitive, quality control strategy that protects multicellular organisms from exposure to immature and damaged secretory proteins. So at the cost of losing some cells, multicellular organisms may benefit temporarily from Terminal UPR-induced apoptosis. However, many deadly human diseases occur if too many cells die through this process. Conversely, many human diseases such as diabetes mellitus and retinopathies proceed from unchecked cell degeneration under ER stress (Merksamer, P. I., and Papa, F. R., J Cell Sci 123, 1003-1006 (2010); Papa, F. R. Cold Spring Harbor perspectives in medicine 2, a007666 (2012); Shore, G. C., Papa, F. R., and Oakes, S. A., Curr Opin Cell Biol 23, 143-149 (2011)). Type 2 diabetes may be a prototype of cell degenerative diseases caused by UPR-mediated apoptosis under irremediable ER stress. These same principles appear to be at play in type 1 diabetes, wherein immune attack on islet β-cells elevates ER workload and causes ER stress in remaining cells. A deeper fundamental and mechanistic understanding of the etiology and pathogenesis of diabetes mellitus may lead to increasing opportunities for the development of novel and effective therapies. Terminal UPR signaling is central to these conditions as shown through experimental data and uses of proprietary compounds that defeat the consequences of terminal UPR signaling in ER stress-challenged β-cells to afford significant cytoprotection.

IRE1α and IRE1β are ER-transmembrane proteins that become activated when unfolded proteins accumulate within the organelle. IRE1α is the more widely expressed and well-studied family member. The bifunctional kinase/endoribonuclease IRE1α controls entry into the terminal UPR. IRE1α senses unfolded proteins through an ER lumenal domain that becomes oligomerized during stress (Zhou, J. et al. Proceedings of the National Academy of Sciences of the United States of America 103, 14343-14348 (2006); Credle, J. J. et al. Proc Natl Acad Sci USA 102, 18773-18784 (2005); Aragon, T. et al. Nature (2008); Aragon, T. et al. Nature 457, 736-740 (2009)). On its cytosolic face, IRE1α possesses bifunctional kinase/RNase activities. Oligomerization juxtaposes IRE1α's kinase domains, which consequently trans-autophosphorylate. Kinase autophosphorylation activates the RNase activity, which cleaves XBP1 mRNA at specific sites to excise an intron. Religation of IRE1α-cleaved XBP1 mRNA shifts the open reading frame; translation of spliced XBP1 mRNA produces a transcription factor called XBP1s (s=spliced) (Calfon, M. et al. Nature 415, 92-96, (2002); Yoshida, H. Cell 107, 881-891 (2001)). XBP1s's target genes encode products that enhance ER protein folding and quality control (Lee, A. H. et al., Molecular and cellular biology 23, 7448-7459 (2003)). Thus, IRE1α promotes adaptation via XBP1s.

Under irremediable ER stress, positive feedback signals emanate from the UPR and become integrated and amplified at key nodes to trigger apoptosis. IRE1α is a key initiator of these pro-apoptotic signals. IRE1α employs auto-phosphorylation as a “timer.” Remediable ER stress causes low-level, transient auto-phosphorylation that confines RNase activity to XBP1 mRNA splicing. However, sustained kinase autophosphorylation causes IRE1α's RNase to acquire relaxed specificity, causing it to endonucleolytically degrade thousands of ER-localized mRNAs in close proximity to IRE1α (Han, D. et al. Cell 138, 562-575, (2009); Hollien, J. et al. Journal of Cell Biology. These mRNAs encode secretory proteins being co-translationally translocated (e.g., insulin in β cells). As mRNA degradation continues, transcripts encoding ER-resident enzymes also become depleted, thus destabilizing the entire ER protein-folding machinery. Once IRE1α's RNase becomes hyperactive, adaptive signaling through XBP1 splicing becomes eclipsed by ER mRNA destruction, which pushes cells into apoptosis.

A terminal UPR signature tightly controlled by IRE1α's hyperactive RNase activity causes (1) widespread mRNA degradation at the ER membrane that leads to mitochondrial apoptosis (Han, D. et al. Cell 138, 562-575, (2009)), (2) induction of the pro-oxidant thioredoxin-interacting protein (TXNIP), which activates the NLRP3 inflammasome to produce maturation and secretion of interleukin-1β, and consequent sterile inflammation in pancreatic islets leading to diabetes (Lerner, A. G. et al. Cell metabolism 16, 250-264, (2012)), and (3) degradation of pre-miRNA 17, leading to translational upregulation and cleavage of pre-mitochondrial caspase 2 (Upton, J. P. et al. Science 338, 818-822, (2012)) and stabilization of the mRNA encoding TXNIP (Lemer, A. G. et al. Cell metabolism 16, 250-264, (2012)).

Retinitis pigmentosa (RP) is a clinically and genetically heterogeneous group of inherited retinal disorders characterized by diffuse progressive dysfunction and loss of rod and cone photoreceptors, and retinal pigment epithelium. There are no approved therapies to offer the over 100,000 Americans who currently suffer from RP. As RP is a leading cause of irreversible vision loss, new therapeutic approaches for this condition would be expected to have significant cost-saving benefits for health care systems.

A great deal of evidence suggests that the accumulation of misfolded proteins within the ER is a central causative mechanism in many forms of RP. When the protein-folding capacity of the ER is overwhelmed, cells experience “ER stress” and actively commit programmed cell death. For example, mutations in rhodopsin are the most common cause of RP in the US and lead to a defective rhodopsin protein that misfolds and accumulates in the ER to cause high levels ER stress. Here we show that multiple Terminal UPR outputs, including cell proliferation blocks, sterile inflammation, and apoptosis result from kinase-driven increases in the oligomerization state of IRE1α's cytosolic domains that hyperactivate its RNase. These destructive events are prevented by breaking IRE1α oligomerization through mutations that are either rationally designed or occur somatically in the Ire1α gene of human cancers. To test physiological effects of pharmacologically inhibiting IRE1α, we developed small molecule kinase inhibitors that prevent oligomerization and allosterically inhibit its RNase. One such IRE1α kinase inhibitor preserves viability and function in ER-stressed cells, pancreatic islet explants, and rodent models of ER stress-induced retinitis pigmentosa and diabetes.

Disclosed herein, inter alia, are solutions to these and other problems in the art.

### BRIEF SUMMARY

Provided herein, inter alia, are novel ATP-competitive small molecule kinase inhibitors of IRE1α that prevent oligomerization and/or allosterically inhibit its RNase activity.

In an aspect is provided a method of treating a disease in a patient in need of such treatment, the method including administering a first amount of an Ire1 (e.g., Ire1α) kinase modulating compound, or a pharmaceutically acceptable salt thereof, and a second amount of an Ire1 (e.g., Ire1α) ribonuclease modulating compound, or a pharmaceutically acceptable salt thereof, to the patient, wherein the disease is a neurodegenerative disease, demyelinating disease, cancer, eye disease, fibrotic disease, or diabetes, wherein the first amount and the second amount are together a combined synergistic amount.

In another aspect is provided a method of modulating the activity of an Ire1 (e.g., Ire1α) protein, the method including contacting the Ire1 (e.g., Ire1α) protein with an Ire1 (e.g., Ire1α) kinase modulating compound, or a pharmaceutically acceptable salt thereof, and an Ire1 (e.g., Ire1α) ribonuclease modulating compound, or a pharmaceutically acceptable salt thereof.

In an aspect is provided a pharmaceutical combination including a first amount of an Ire1 (e.g., Ire1α) kinase modulating compound and a second amount of an Ire1 (e.g., Ire1α) ribonuclease modulating compound, wherein the first amount and the second amount are together a combined synergistic amount.

### BRIEF DESCRIPTION OF THE DRAWINGS

FIGS. 1A-1C. Interaction of ATP-competitive inhibitors with the bifunctional kinase/RNase, IRE1α. (FIG. 1A) XBP1 RNA minisubstrate assay used for screening IRE1α modulators; the recombinant human IRE1α-IRE1α*-used in the assay spans residues 469-977, which includes the cytosolic kinase and RNase domains; cleavage of the 5′FAM-3′BHQ-labeled XBP1 minisubstrate by IRE1α* results in FRET-dequenching. (FIG. 1B) Endpoint fluorescence of IRE1α* catalyzed cleavage reaction of XBP1 minisubstrate in the presence of varying concentrations of inhibitors, or DMSO; STF-083010 is an imine-based compound that covalently inhibits the RNase domain; relative fluorescence intensity is scaled to the signal observed with IRE1α* (1.0), or without IRE1α* (0). (mean±SD, n=3). (FIG. 1C) Structures of type II kinase inhibitors that inhibit the RNase activity of IRE1α* (GP118/KIRA1, GP117/KIRA2, GP146/KIRA3). Numbering of amino acid residues from Ire1α in figures uses amino acid numbering of human Ire1α (e.g. including numbering used for human IRE1α (469-977) sequence in SEQ ID NO:2 as numbered in the Examples section herein).

FIGS. 2A-2E. APY29 and GP146 (KIRA3) divergently modulate the RNase activity and oligomerization state of IRE1α*. (FIG. 2A) Inhibition of IRE1α* autophosphorylation in vitro by APY29 and GP146; top panels show autoradiograms of autophosphorylation levels under serial two-fold dilutions of the respective inhibitors (from 80 μM to 0.0098 μM); the lower panel shows normalized autophosphorylation levels and IC₅₀ values for both compounds. (FIG. 2B) X-PPase treatment of IRE1α* produces dephosphorylated IRE1α* (dP-IRE1α*); immunoblots using anti-IRE1α and anti-phospho IRE1α antibodies are shown. (FIG. 2C) RNase activities of IRE1α* and dP-IRE1α* under varying [APY29] or [GP146] (KIRA3) per the assay of FIG. 1b; EC₅₀ values were determined by fitting normalized fluorescence intensities (mean±SD, n=3). (FIG. 2D) Urea PAGE of XBP1 mini-substrate cleavage by IRE1α* and dP-IRE1α* with and without GP146 (KIRA3) or APY29. (FIG. 2E) RNase competition assays between APY29 and GP146 (KIRA3); the line marked with circles shows IRE1α* RNAse activity under fixed GP146 (KIRA3) and varying [APY29]; the line marked with squares shows IRE1α* RNAse activity under fixed APY29 and varying [GP146] (KIRA3); the line marked with triangles shows IRE1α* RNAse activity under fixed STF-083010 and varying [APY29] (mean±SD, n=3).

FIGS. 3A-3D. Characterization of GP146's interaction with the ATP-binding site of IRE1α. (FIG. 3A) A crystal structure of the kinase domain of human IRE1α bound to ADP; the native cysteine residues that were monitored using the ICAT footprinting method are labeled and shown as thick rods and the DFG-motif is shown as thin bars; cys715 is part of the hinge region of IRE1α and its side chain partially occupies the ATP-binding site; cys645 is in the activation loop, two residues away from the DFG-motif; cys572 is located on the top of the N-terminal lobe of the catalytic domain and is distant from the ATP-binding site. (FIG. 3B) Results of the ICAT footprinting experiments with IRE1α*; alkylation rates were measured in the presence of DMSO (circle), APY29 (square) (20 μM), or GP146 (triangle) (20 M) (mean±SD, n=3). (FIG. 3C) A molecular model of GP146's interaction with the ATP-binding site of IRE1α; IRE1α is in the DFG-out inactive conformation; the imidazopyrazine ring of GP146 occupies the adenine pocket and the 3-trifluoromethylurea occupies the DFG-out pocket; no favorable poses for GP146 bound to the DFG-in conformation of IRE1α could be determined. (FIG. 3D) Control compounds that were generated to test the docked structure of GP146 bound to the DFG-out conformation of IRE1α; GP146(NMe) contains a methyl group that is predicted to disrupt a critical hydrogen bond to the hinge region of IRE1α GP146(Am) contains an amide rather than a urea group linker between the naphthyl ring and the trifluormethylpheny group; the amide linker is predicted to lead to a less favorable interaction with the DFG-out pocket; the IC₅₀s for each compound against IRE1α* is listed below their structure.

FIGS. 4A-4B APY29 and GP146 differentially affect oligomerization state of IRE1α*. (FIG. 4A) Left panels shows immunoblots of IRE1α* after treatment with the crosslinker DSS (250 μM); increasing concentrations of IRE1α* were incubated with DMSO, APY29 (200 μM), or GP146 (200 μM); the right panel shows quantitation of the ratios of oligomeric to monomeric IRE1α*. (FIG. 4B) Model of how type I and type II kinase inhibitors affect the RNase activities and oligomeric states of IRE1α* and dP-IRE1α*.

FIGS. 5A-5E. Divergent modulation of endogenous IRE1α RNase activity under ER stress with types I and II kinase inhibitors. (FIG. 5A) EtBr-stained agarose gel of XBP1 complementary DNA (cDNA) amplicons from INS-1 cells pre-treated for 1 hr with GP146 or APY29 at indicated concentrations, followed by thapsigargin (Tg) (6 nM) for 4 hrs; ratios of spliced XBP1 (XBP1S) over (spliced+unspliced (XBP1U)) are plotted (mean±SD, n=3). (FIG. 5B) Anti-total and anti-phospho IRE1α immunoblots using extracts from INS-1 cells pre-treated for 1 hr with GP146, sunitinib, or STF-083010 at indicated concentrations, followed by Tg (6 nM) for 2 hrs. (FIG. 5C) EtBr-stained agarose gel of XBP1 complementary DNA (cDNA) amplicons from the INS-1 cells described in (B). (FIG. 5D) EtBr-stained agarose gel of XBP1 complementary DNA (cDNA) amplicons from INS-1 cells pre-treated for 1 hr with GP146(NMe) at indicated concentrations, followed by thapsigargin (Tg) (6 nM) for 4 hrs. (FIG. 5E) Model of how type I kinase inhibitors (APY29), type II kinase inhibitors (GP146), and RNase inhibtors (STF-083010) modulate the enzymatic activities of WT IRE1α. APY29 inhibits IRE1α trans-autophosphorylation but promotes oligomerization and activates the RNase domain; STF-083010 inhibits the RNase activity of IRE1α but does not affect kinase activity or the overall oligomerization state. GP146 inhibits both the kinase and RNase domains of IRE1α and stabilizes the monomeric form; cartoons are not meant to differentiate between the relative orientations of monomer subunits in IRE1α.

FIG. 6 illustrates analogs of GP146 that demonstrate the ability to modulate and/or inhibit IRE1α RNase activity.

FIGS. 7A-7I. Direct inhibition of IRE1α RNase prevents IRE1 dependent ER-localized mRNA degradation and ER stress-induced apoptosis. (FIG. 7A) Model of inhibition of IRE1α RNase activity by STF-083010 (STF). (FIG. 7B) Percent XBP1 splicing in INS-1 IRE1 WT stable cells treated with 5 ng/mL Dox and 50 μM STF for indicated times as shown (upper panel). EtBr-stained agarose gel of XBP1 cDNA amplicons is shown for the same samples above (lower panel). (FIG. 7C) Q-PCR for Insulin1 mRNA (normalized to GAPDH) in INS-1 IRE1 WT stable cells treated Dox and STF for 12, 24, 48 and 72 h. (FIG. 7D) Anti-Phospho-IRE1α and Anti-Total IRE1α immunoblots of INS-1 IRE1 WT stable cells treated for 48 h with 5 ng/mL Dox and 50 μM STF. (FIG. 7E) Anti-Phospho and Total JNK immunoblots of same samples. (FIG. 7F) Anti-Pro Caspase and Cleaved Caspase-3 immunoblots of same samples. (FIG. 7G) Percent of INS-1 IRE1 WT stable cells staining positive for Annexin V after treatment for 72 h with Dox and STF as indicated. (FIG. 7H) Percent of INS-1 cells staining positive for Annexin V after treatment for 72 h with increasing doses of Tunicamycin (Tm) and 50 μM STF as indicated. (FIG. 7I) Immunofluorescence staining on islets from 10 week old C57BL6 mice treated with 0.5 μg/mL Tm and 50 μM STF for 16 h as indicated; co-stained for DAPI (left column), insulin (second column from left), and TUNEL (third column from left); merged image is also shown. (FIG. 7J) Quantification of TUNEL positive β-cells normalized to DAPI-positive cells in (I).

FIGS. 8A-8E. (FIG. 8A) Percent of INS-1 cells staining positive for Annexin-V 72 hrs after treatment of 500 ng/ml Tm+/−GP165. (FIG. 8B) EtBr-stained agarose gel of XBP1 cDNA amplicons from INS-1 cells 8 hrs after treatment of 200 ng/ml Tm+/−GP165. XBP1U, unspliced XBP1; XBP1S, spliced XBP1; the lower panel shows the ratios of spliced XBP1 (XBP1 S) over (spliced+unspliced (XBP1U)). (FIG. 8C) Immunoblots for CASP3 cleavage in INS-1 cells 72 hrs after treatment of 500 ng/ml Tm+/−GP165 or 50 μM STF-083010 as positive control. (FIG. 8D) qPCR of insulin mRNA in INS-1 cells 8 hrs after treatment of 500 ng/ml Tm+/−GP165. (FIG. 8E) A schematic model of how GP165 blocks terminal UPR by inhibiting IRE1α activation under ER stress; as a type II kinase inhibitor, GP165 binds to the adenosine binding pocket and inhibits both the kinase and RNase domains of IRE1α and stabilizes the monomeric form; similar results are obtained using GP146. GP165 is KIRA6.

FIG. 9. Coomassie blue-stained PAGE of purified IRE1α*; M, protein marker.

FIG. 10. Structures of several type II kinase inhibitors screened against IRE1α* in the XBP1 RNA minisubstrate assay; the relative endpoint fluorescence intensities for the IRE1α*-catalyzed cleavage reaction of XBP1 minisubstrate in the presence of varying concentrations of inhibitors are shown.

FIGS. 11A-11C. Sunitinib inhibits IRE1α* autophosphorylation but activates the RNase domain. (FIG. 11A) Autoradiograms of IRE1α* autophosphorylation levels under serial two-fold dilutions of sunitinib (from 80 M to 0.0098 M). (FIG. 11B) Urea PAGE analysis of XBP1 minisubstrate cleavage by IRE1α* and dP-IRE1α* with and without sunitinib. (FIG. 11C) Urea PAGE analysis of XBP1 minisubstrate cleavage by IRE1α* with fixed GP146 (10 μM) and varying sunitinib concentrations.

FIG. 12. Synthetic strategy for generating KIRAs.

FIG. 13. Representative KIRAs synthesized and tested.

FIGS. 14A-14B. (FIG. 14A) General structure of irreversible KIRAs that target a cysteine residue located in the activation loop of IRE1; representative electrophiles are shown. (FIG. 14B) A close-up of the ATP-binding site of IRE1α.

FIGS. 15A-15K. KIRA6 inhibits IRE1α autophosphorylation, breaks oligomers, reduces RNase activity, and protects cells from entry into apoptosis. (FIG. 15A) Structure of KIRA6. (FIG. 15B) RNase activities of IRE1α* under varying [KIRA6]; half-maximum effective concentration (EC₅₀) values were determined by fitting normalized fluorescence intensities (mean±s.d., n=3). (FIG. 15C) Inhibition of IRE1α* kinase activity in vitro by KIRA6; IC₅₀ values were determined by fitting percent phosphorylation. (FIG. 15D) In vivo, KIRA6 inhibits endogenous IRE1α auto-phosphorylation in a dose-dependent manner; in contrast the aldehyde-based IRE1α RNase-inhibitor, STF, does not inhibit IRE1α auto-phosphorylation, nor does a control compound KIRA6(in). (FIG. 15E) Immunoblots of IRE1α* (WT) incubated with DMSO or KIRA6 (200 μM) followed by treatment with the crosslinker DSS (250 μM); quantification is on the right. (FIG. 15F) Agarose gel of XBP1 cDNA amplicons from INS-1 cells pre-treated with indicated concentrations of KIRA6 for 1 h, followed by 0.5 μg/ml Tm for 8 h. (FIG. 15G) KIRA6 inhibits ER-localized endonucleolytic decay of Ins1 mRNA at lower doses of the drug than needed to inhibit XBP1 mRNA splicing. (FIG. 15H) KIRAs reduce entry of INS1-1 cells into apoptosis. (FIG. 15I) Immunofluorescence of islets from 10 wk old C57BL/6 mice treated with 0.5 μg/mL Tm plus/minus 0.5 M KIRA6 for 16 hr. Co-stained for DAPI, insulin, and TUNEL; Quantification of TUNEL positive β-cells (white arrows) normalized to DAPI-positive cells is shown. (FIG. 15J) KIRA6 cytoprotective effects are dependent on IRE1α because they are absent in Ire1α^{-/-} mouse embryonic fibroblasts (MEFs), but still demonstrable in WT and Xbp1^{-/-} MEFs. (FIG. 15K) Model of how KIRA6 prevents the terminal UPR by inhibiting IRE1α.

FIGS. 16A-16C. Divergent modulation of IRE1α RNase activity using distinct classes of kinase inhibitors. (FIG. 16A) KIRA6 inhibition of IRE1α* kinase activity. IC₅₀ values determined by fitting percent kinase activity (n=3); Urea PAGE of competition cleavage by IRE1α* of XBP1 RNA mini-substrate (FIG. 16B) and α³²P-labeled Ins2 RNA (FIG. 16C), under indicated [KIRA6]; IC₅₀s determined by fitting in-gel fluorescence intensities (XBP1) and phosphorimager (Ins2).

FIGS. 17A-17C. Systemic KIRA6 attenuates β-cell functional loss, increases insulin levels, and ameliorates hyperglycemia in the Akita mouse. (FIGS. 17A-17B) Random insulin and C-peptide levels in Ins2^{+/Akita} mice on Day 58 (21 d post injections). KIRA6 (5 mg/kg)(n=5) and vehicle (n=4). (FIG. 17C) Total β-cell area as a percentage of total pancreas area on Day 55 (18 d post injections). KIRA6 (5 mg/kg)(n=6) and vehicle (n=3).

FIG. 18. KIRA6 and 1NM-PP1 have opposing effects on IRE1α (I642G); Table showing IC₅₀ values of kinase inhibitory activity of KIRA6 against a panel of 7 indicated kinases in vitro.

FIGS. 19A-19D. KIRA6 inhibits Terminal UPR outputs of IRE1α to protect against ER stress-induced apoptosis. (FIG. 19A) Structure of STF-083010 and cartoon showing that it directly inhibits the RNase of IRE1α (through covalent modification). (FIG. 19B) Q-PCR for Ins1 mRNA in INS-1 IRE1α (WT) cells treated with Dox (5 ng/ml)−/+STF-083010 (50 M) over the indicated timecourse. (FIG. 19C) Annexin V staining of INS-1 cells after 72 hr with indicated [Tm]−/+STF-083010 (50 M). (FIG. 19D) Annexin V staining of INS-1 cells after 72 hr with Tm (0.2 μg/ml), STF-083010 (1 or 5 M), and KIRA6 (0.05 M) as indicated. Data plotted as mean+/−SD. P-values: *<0.05, **<0.01, ***<0.005.

### DETAILED DESCRIPTION

Activation of IRE1α's RNase is normally dependent on kinase autophosphorylation (Tirasophon, W. et al. Genes Dev 12, 1812-1824 (1998)), but an allosteric relationship between these two domains exists, which allows nucleotides (ADP and ATP) and small molecule inhibitors that stabilize an active ATP-binding site conformation to directly activate the RNase without autophosphorylation (Papa, F. R. et al. Science 302, 1533-1537 (2003); Han, D. et al. Biochemical and biophysical research communications 365, 777-783, (2008); Korennykh, A. V. et al. BMC biology 9, 48, (2011)). Furthermore, a particular class of kinase inhibitors (called type II) stabilize an inactive ATP-binding site conformation of IRE1α and are able to potently inhibit its RNase activity by breaking high-order oligomerization state (Wang, L. et al. Nature chemical biology 8, 982-989, (2012)). These compounds are herein labeled—KIRAs—for kinase-inhibiting RNase-attenuators.

Distinct classes of ATP-competitive kinase inhibitors divergently modulate the RNase activity of IRE1α. A co-crystal structure of yeast IRE1 bound with APY29—a predicted type I kinase inhibitor—shows that the kinase catalytic domain is in an active conformation, which is a conformation typically adopted by protein kinases when bound to ATP and other type I inhibitors (Korennykh, A. V. et al. Nature 457, 687-693 (2009); Korennykh, A. V. et al. BMC Biol. 9, 48 (2011)). Moreover, two additional co-crystal structures of yeast IRE1 and human IRE1α bound with ADP show that the kinase domain is similarly in an active conformation (Ali, M. M. et al. EMBO J. 30, 894-905 (2011); Lee, K. P. et al. Cell 132, 89-100 (2008)). By stabilizing IRE1α's kinase in the active conformation, these type I inhibitors act as ligands that allosterically activate its adjacent RNase domain. It might be possible to stabilize IRE1α's kinase domain in an alternative conformation, and in so doing disable its RNase activity. Use of a class of small molecule kinase inhibitors that have been described to selectively stabilize the inactive conformation of the ATP-binding site (type II inhibitors) for a variety of kinases; examples include the clinically-approved drugs imatinib and sorafenib (Liu, Y. & Gray, N. S. Nat. Chem. Biol. 2, 358-364 (2006); Wan, P. T. et al. Cell 116, 855-867 (2004); Schindler, T. et al. Science 289, 1938-1942 (2000)), provides support for this approach. The inactive ATP-binding site conformation stabilized by type II inhibitors is characterized by outward movement of the catalytically-important Asp-Phe-Gly (DFG) motif, and is therefore called the DFG-out conformation (Liu, Y. & Gray, N. S. Nat. Chem. Biol. 2, 358-364 (2006); Ranjitkar, P. et al. Chem. Biol. 17, 195-206 (2010)). In contrast, in all three co-crystal structures of IRE1 in an active conformation mentioned previously, the kinase domain adopts the DFG-in conformation (Korennykh, A. V. et al. Nature 457, 687-693 (2009); Ali, M. M. et al. EMBO J. 30, 894-905 (2011); Lee, K. P. et al. Cell 132, 89-100 (2008)).

Under high endoplasmic reticulum (ER) stress, hyperactivation of intracellular signaling pathways termed the unfolded protein response (UPR) triggers cell death. Signature events of this “Terminal UPR” are controlled by IRE1α, an ER bifunctional kinase/endoribonuclease (RNase), which, when oligomerized, endonucleolytically degrades ER-localized mRNAs and repressive micro-RNA precursors to trigger apoptosis. Ire1α somatic mutations found in human cancers disable oligomerization and apoptotic function of its RNase. Using these instructive results from human biology, ATP-competitive kinase inhibitors were developed—termed KIRAs (Kinase Inhibiting RNase Attenuators)—that allosterically reduce IRE1α oligomerization and RNase activity. One such kinase inhibitor, KIRA6, inhibits all IRE1α outputs, and preserves cell viability and function under ER stress. In rat models of retinal degeneration caused by ER stress, intravitreal KIRA6 prevents photoreceptor loss.

Artificial ER stress agents are widely used to acutely activate the UPR, but saturating doses that have no natural pathophysiological correlate are often employed. To test cytoprotection later in the work, dose-response regimes were established using three ER stress agents that dose-dependently push rat insulinoma (INS-1) cells, which have a well-developed ER and secrete insulin, past a stress threshold and ultimately, in switch-like manner, into apoptosis. The percentage of INS-1 cells entering apoptosis upon exposure to the ER Ca²⁺ pump inhibitor thapsigargin (Tg) depends aggregately on two variables: concentration and duration of exposure. Similar results hold for the glycosylation inhibitor tunicamycin (Tm) and the anterograde trafficking inhibitor brefeldin A (BFA). Preceding apoptosis, increasing levels of ER stress agents progressively increase IRE1α phosphorylation, XBP1 mRNA splicing, endonucleolytic decay of the ER-localized mRNA, Ins1 mRNA (which encodes proinsulin), induction of thioredoxin-interacting protein (TXNIP) mRNA (whose product activates the NLRP3 inflammasome), and downstream c-Jun terminal kinase phosphorylation (JNKs). All these signature events, culminating in apoptosis, can be simulated, without imposing ER stress, by conditionally over-expressing wild-type (WT) IRE1α in INS-1 stable cell lines (Han et al., 2009). Induced with doxycycline (Dox), the transgenic IRE1α (WT) proteins spontaneously self-associate, trans-autophosphorylate, and trigger XBP1 mRNA splicing (Han et al., 2009). Increasing [Dox] causes progressive decay of Ins1 mRNA, elevation of TXNIP mRNA, and apoptosis. Thus, as with ER stress agents, dose escalation of IRE1α (WT) transgene expression allows graduated control over the Terminal UPR and is sufficient to push cells, in switch-like manner, past an apoptotic threshold. It was previously shown that pre-emptively producing XBP1s, 1NM-PP1-activated IRE1α (I642G) provides a metastable degree of cytoprotection against subsequent ER stress (Han et al., 2009; Han et al., 2008), as does forced expression of XBP1s, shown here. However, without a window of sufficient time to permit adaptive pre-conditioning, simultaneous provision of 1NM-PP1 and ER stress agents rescues Ins1 mRNA decay and apoptosis, in 1NM-PP1 dose-dependent manner. Systemically, KIRA6 preserves pancreatic β-cells, increases insulin, and reduces hyperglycemia in Akita diabetic mice. Thus, IRE1α powerfully controls cell fate, but can itself be controlled with small molecules to reduce cell degeneration.

### A. DEFINITIONS

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—. Terms used herein may be preceded and/or followed by a single dash, “ ”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents having a superscript “d” (e.g. R^{d}) are intended to be read “left to right” unless a dash indicates otherwise. For example, C₁C₆alkoxycarbonyloxy and OC(O)C₁C₆alkyl indicate the same functionality; similarly arylalkyl and -alkylaryl indicate the same functionality.

The term “saturated” as used herein means the referenced chemical structure does not contain any multiple carbon carbon bonds. For example, a saturated cycloalkyl group as defined herein includes cyclohexyl, cyclopropyl, and the like.

The term “alkyl,” by itself or as part of another substituent, means, unless otherwise stated, a non-cyclic straight (i.e., unbranched) or non-cyclic branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). In embodiments, an alkyl is a straight or branched chain hydrocarbon containing from 1 to 10 carbon atoms, unless otherwise specified. In embodiments, an alkyl is an alkenyl, wherein the term “alkenyl” is used in accordance with its plain ordinary meaning. In embodiments, an alkenyl is a straight or branched chain hydrocarbon containing from 2 to 10 carbons, unless otherwise specified, and containing at least one carbon carbon double bond. Examples of alkenyl include, but are not limited to, ethenyl, 2 propenyl, 2 methyl 2 propenyl, 3 butenyl, 4 pentenyl, 5 hexenyl, 2 heptenyl, 2 methyl 1 heptenyl, 3 decenyl, and 3,7 dimethylocta 2,6 dienyl. In embodiments, an alkyl is an alkynyl, wherein the term “alkynyl” is used in accordance with its plain ordinary meaning. In embodiments, an alkynyl is a straight or branched chain hydrocarbon group containing from 2 to 10 carbon atoms and containing at least one carbon carbon triple bond. Examples of alkynyl include, but are not limited, to acetylenyl, 1 propynyl, 2 propynyl, 3 butynyl, 2 pentynyl, and 1 butynyl.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable non-cyclic straight or non-cyclic branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively, wherein the carbons making up the ring or rings do not necessarily need to be bonded to a hydrogen due to all carbon valencies participating in bonds with non-hydrogen atoms. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂), where w is 1, 2, or 3). Representative examples of bicyclic ring systems include, but are not limited to, bicyclo[3.1.1]heptane, bicyclo[2.2.1]heptane, bicyclo[2.2.2] octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)w, where w is 1, 2, or 3). Representative examples of bicyclic cycloalkenyls include, but are not limited to, norbomenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3 dioxanyl, 1,3 dioxolanyl, 1,3 dithiolanyl, 1,3 dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1 dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3 dihydrobenzofuran 2 yl, 2,3 dihydrobenzofuran 3 yl, indolin 1 yl, indolin 2 yl, indolin 3 yl, 2,3 dihydrobenzothien 2 yl, decahydroquinolinyl, decahydroisoquinolinyl, octahydro 1H indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo [b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively, such as for example a divalent radical of indoline. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

In embodiments, an aryl is a phenyl (i.e., monocyclic aryl), a bicyclic ring system containing at least one phenyl ring or an aromatic bicyclic ring containing only carbon atoms in the aromatic bicyclic ring system or a multicyclic aryl ring system, provided that the bicyclic or multicyclic aryl ring system does not contain a heteroaryl ring when fully aromatic. In embodiments, the bicyclic aryl can be azulenyl, naphthyl, or a phenyl fused to a monocyclic cycloalkyl, a monocyclic cycloalkenyl, or a monocyclic heterocyclyl. In embodiments, the bicyclic aryl may be attached to the parent molecular moiety through any carbon atom contained within the phenyl portion of the bicyclic system, or any carbon atom with the napthyl or azulenyl ring. In embodiments, the fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden 1 yl, dihydroinden 2 yl, dihydroinden 3 yl, dihydroinden 4 yl, 2,3 dihydroindol 4 yl, 2,3 dihydroindol 5 yl, 2,3 dihydroindol 6 yl, 2,3 dihydroindol 7 yl, inden 1 yl, inden 2 yl, inden 3 yl, inden 4 yl, dihydronaphthalen 2 yl, dihydronaphthalen 3 yl, dihydronaphthalen 4 yl, dihydronaphthalen 1 yl, 5,6,7,8 tetrahydronaphthalen 1 yl, 5,6,7,8 tetrahydronaphthalen 2 yl, 2,3 dihydrobenzofuran 4 yl, 2,3 dihydrobenzofuran 5 yl, 2,3 dihydrobenzofuran 6 yl, 2,3 dihydrobenzofuran 7 yl, benzo[d][1,3]dioxol 4 yl, benzo[d][1,3]dioxol 5 yl, 2H chromen 2 on 5 yl, 2H chromen 2 on 6 yl, 2H chromen 2 on 7 yl, 2H chromen 2 on 8 yl, isoindoline 1,3 dion 4 yl, isoindoline 1,3 dion 5 yl, inden 1 on 4 yl, inden 1 on 5 yl, inden 1 on 6 yl, inden 1 on 7 yl, 2,3 dihydrobenzo[b][1,4]dioxan 5 yl, 2,3 dihydrobenzo[b][1,4]dioxan 6 yl, 2H benzo[b][1,4]oxazin3(4H) on 5 yl, 2H benzo[b][1,4]oxazin3(4H) on 6 yl, 2H benzo[b][1,4]oxazin3(4H) on 7 yl, 2H benzo[b][1,4]oxazin3(4H) on 8 yl, benzo[d]oxazin 2(3H) on 5 yl, benzo[d]oxazin 2(3H) on 6 yl, benzo[d]oxazin 2(3H) on 7 yl, benzo[d]oxazin 2(3H) on 8 yl, quinazolin 4(3H) on 5 yl, quinazolin 4(3H) on 6 yl, quinazolin 4(3H) on 7 yl, quinazolin 4(3H) on 8 yl, quinoxalin 2(1H) on 5 yl, quinoxalin 2(1H) on 6 yl, quinoxalin 2(1H) on 7 yl, quinoxalin 2(1H) on 8 yl, benzo[d]thiazol 2(3H) on 4 yl, benzo[d]thiazol 2(3H) on 5 yl, benzo[d]thiazol 2(3H) on 6 yl, and, benzo[d]thiazol 2(3H) on 7 yl. In embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia. In embodiments, multicyclic aryl groups are a phenyl ring (base ring) fused to either (i) one ring system selected from the group consisting of a bicyclic aryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic cycloalkyl, a monocyclic or bicyclic cycloalkenyl, and a monocyclic or bicyclic heterocyclyl, provided that when the base ring is fused to a bicyclic cycloalkyl, bicyclic cycloalkenyl, or bicyclic heterocyclyl, then the base ring is fused to the base ring of the bicyclic cycloalkyl, bicyclic cycloalkenyl, or bicyclic heterocyclyl. In embodiments, the multicyclic aryl may be attached to the parent molecular moiety through any carbon atom contained within the base ring. In certain embodiments, multicyclic aryl groups are a phenyl ring (base ring) fused to either (i) one ring system selected from the group consisting of a bicyclic aryl, a bicyclic cycloalkyl, a bicyclic cycloalkenyl, and a bicyclic heterocyclyl; or (ii) two other ring systems independently selected from the group consisting of a phenyl, a monocyclic cycloalkyl, a monocyclic cycloalkenyl, and a monocyclic heterocyclyl, provided that when the base ring is fused to a bicyclic cycloalkyl, bicyclic cycloalkenyl, or bicyclic heterocyclyl, then the base ring is fused to the base ring of the bicyclic cycloalkyl, bicyclic cycloalkenyl, or bicyclic heterocyclyl. Examples of multicyclic aryl groups include but are not limited to anthracen-9-yl and phenanthren-9-yl.

In embodiments, the term “heteroaryl,” as used herein, means a monocyclic, bicyclic, or a multicyclic heteroaryl ring system. In embodiments, the monocyclic heteroaryl can be a 5 or 6 membered ring. In embodiments, the 5 membered ring consists of two double bonds and one, two, three or four nitrogen atoms and optionally one oxygen or sulfur atom. In embodiments, the 6 membered ring consists of three double bonds and one, two, three or four nitrogen atoms. In embodiments, the 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. In embodiments, the bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. In embodiments, the fused cycloalkyl or heterocyclyl portion of the bicyclic heteroaryl group is optionally substituted with one or two groups which are independently oxo or thia. In embodiments, when the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. In embodiments, when the bicyclic heteroaryl is a monocyclic heteroaryl fused to a phenyl ring or a monocyclic heteroaryl, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6 dihydroquinolin 2 yl, 5,6 dihydroisoquinolin 1 yl, furopyridinyl, indazolyl, indolyl, isoquinolinyl, naphthyridinyl, quinolinyl, purinyl, 5,6,7,8 tetrahydroquinolin 2 yl, 5,6,7,8 tetrahydroquinolin 3 yl, 5,6,7,8 tetrahydroquinolin 4 yl, 5,6,7,8 tetrahydroisoquinolin 1 yl, thienopyridinyl, 4,5,6,7 tetrahydrobenzo[c][1,2,5]oxadiazolyl, and 6,7 dihydrobenzo[c][1,2,5]oxadiazol 4(5H) onyl. In embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia. In embodiments, the multicyclic heteroaryl group is a monocyclic heteroaryl ring (base ring) fused to either (i) one ring system selected from the group consisting of a bicyclic aryl, a bicyclic heteroaryl, a bicyclic heterocyclyl, a bicyclic cycloalkenyl, and a bicyclic cycloalkyl; or (ii) two ring systems selected from the group consisting of a phenyl, a bicyclic aryl, a monocyclic or bicyclic heteroaryl, a monocyclic or bicyclic heterocyclyl, a monocyclic or bicyclic cycloalkenyl, and a monocyclic or bicyclic cycloalkyl. In embodiments, the multicyclic heteroaryl group is connected to the parent molecular moiety through any carbon atom or nitrogen atom contained within the base ring. In embodiments, multicyclic heteroaryl groups are a monocyclic heteroaryl ring (base ring) fused to either (i) one ring system selected from the group consisting of a bicyclic aryl, a bicyclic heteroaryl, a bicyclic heterocyclyl, a bicyclic cycloalkenyl, and a bicyclic cycloalkyl; or (ii) two ring systems selected from the group consisting of a phenyl, a monocyclic heteroaryl, a monocyclic heterocyclyl, a monocyclic cycloalkenyl, and a monocyclic cycloalkyl. Examples of multicyclic heteroaryls include, but are not limited to 5H-[1,2,4]triazino[5,6-b]indol-5-yl, 2,3,4,9-tetrahydro-1H-carbazol-9-yl, 9H-pyrido[3,4-b]indol-9-yl, 9H-carbazol-9-yl, acridin-9-yl,

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “thia” as used herein means a ═S group.

The term “alkylsulfonyl,” as used herein, means a moiety having the formula —S(O)₂—R′, where R′ is a substituted or unsubstituted alkyl group as defined above. R′ may have a specified number of carbons (e.g., “C₁-C₄ alkylsulfonyl”).

The term “arylalkyl” and “-alkylaryl” as used herein, means an aryl group, as defined herein, appended to the parent molecular moiety through an alkyl group, as defined herein. Representative examples of arylalkyl include, but are not limited to, benzyl, 2 phenylethyl, 3 phenylpropyl, and 2 naphth 2 ylethyl.

The term “heteroarylalkyl” and “-alkylheteroaryl” as used herein, means a heteroaryl, as defined herein, appended to the parent molecular moiety through an alkyl group, as defined herein. Representative examples of heteroarylalkyl include, but are not limited to, fur 3 ylmethyl, 1H imidazol 2 ylmethyl, 1H imidazol 4 ylmethyl, 1 (pyridin 4 yl)ethyl, pyridin 3 ylmethyl, pyridin 4 ylmethyl, pyrimidin 5 ylmethyl, 2 (pyrimidin 2 yl)propyl, thien 2 ylmethyl, and thien 3 ylmethyl.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR′R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section below.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)-for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat cancer (e.g. multiple myeloma, cancers of secretory cells), neurodegenerative diseases, demyelinating diseases, eye diseases, fibrotic diseases, or diabetes (type I or type II). For example certain methods herein treat cancer by decreasing or reducing or preventing the occurrence, growth, metastasis, or progression of cancer; treat neurodegeneration by improving mental wellbeing, increasing mental function, slowing the decrease of mental function, decreasing dementia, delaying the onset of dementia, improving cognitive skills, decreasing the loss of cognitive skills, improving memory, decreasing the degradation of memory, or extending survival; treat demyelinating diseases by reducing a symptom of demyelinating diseases or reducing the loss of myelin or increasing the amount of myelin or increasing the level of myelin; treat diabetes by decreasing a symptom of diabetes or decreasing loss of insulin producing cells or decreasing loss of pancreatic cells or reducing insulin insensitivity; treat cancer by decreasing a symptom of cancer, or treat neurodegeneration by treating a symptom of neurodegeneration. Symptoms of cancer (e.g. multiple myeloma, cancers of secretory cells), neurodegenerative diseases, demyelinating diseases, eye diseases, fibrotic diseases, or diabetes would be known or may be determined by a person of ordinary skill in the art. The term “treating” and conjugations thereof, include prevention of an injury, pathology, condition, or disease (e.g. preventing the development of one or more symptoms of cancer, neurodegenerative diseases, demyelinating diseases, and/or diabetes).

An “effective amount” is an amount sufficient to accomplish a stated purpose (e.g. achieve the effect for which it is administered, treat a disease, reduce enzyme activity, increase enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein relative to the absence of the antagonist. An “activity increasing amount,” as used herein, refers to an amount of agonist (activator) required to increase the activity of an enzyme or protein relative to the absence of the agonist. A “function disrupting amount,” as used herein, refers to the amount of antagonist (inhibitor) required to disrupt the function of an enzyme or protein relative to the absence of the antagonist. A “function increasing amount,” as used herein, refers to the amount of agonist (activator) required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g cancer (e.g. multiple myeloma, cancers of secretory cells), neurodegenerative diseases, demyelinating diseases, eye diseases, fibrotic diseases, or diabetes) means that the disease (e.g cancer (e.g. multiple myeloma, cancers of secretory cells), neurodegenerative diseases, demyelinating diseases, eye diseases, fibrotic diseases, or diabetes) is caused by (in whole or in part), or a symptom of the disease is caused by (in whole or in part) the substance or substance activity or function. For example, a symptom of a disease or condition associated with an increase in Ire1 (e.g. Ire1α) activity may be a symptom that results (entirely or partially) from an increase in Ire1 (e.g. Ire1α) activity (e.g increase in Ire1 (e.g. Ire1α) phosphorylation or activity of phosphorylated Ire1 (e.g. Ire1α) or activity of Ire1 (e.g. Ire1α) or increase in activity of an Ire1 (e.g. Ire1α) signal transduction or signalling pathway, Ire1 (e.g. Ire1α) RNase activity). As used herein, what is described as being associated with a disease, if a causative agent, could be a target for treatment of the disease. For example, a disease associated with increased Ire1 (e.g. Ire1α) activity or Ire1 (e.g. Ire1α) pathway activity (e.g. phosphorylated Ire1 (e.g. Ire1α) activity or pathway), may be treated with an agent (e.g. compound as described herein) effective for decreasing the level of activity of Ire1 (e.g. Ire1α) activity or Ire1 (e.g. Ire1α) pathway or phosphorylated Ire1 (e.g. Ire1α) activity or pathway. For example, a disease associated with phosphorylated Ire1 (e.g. Ire1α), may be treated with an agent (e.g. compound as described herein) effective for decreasing the level of activity of phosphorylated Ire1 (e.g. Ire1α) or a downstream component or effector of phosphorylated Ire1 (e.g. Ire1α). For example, a disease associated with Ire1 (e.g. Ire1α), may be treated with an agent (e.g. compound as described herein) effective for decreasing the level of activity of Ire1 (e.g. Ire1α) or a downstream component or effector of Ire1 (e.g. Ire1α).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. Ire1 (e.g. Ire1α) or phosphorylated Ire1 (e.g. Ire1α) or component of Ire1 (e.g. Ire1α) pathway or component of phosphorylated Ire1 (e.g. Ire1α) pathway). In some embodiments contacting includes allowing a compound described herein to interact with a protein or enzyme that is involved in a signaling pathway (e.g. Ire1 (e.g. Ire1α) protein or Ire1 (e.g. Ire1α) pathway).

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a signal transduction pathway or signaling pathway (e.g. Ire1 (e.g. Ire1α) or phosphorylated Ire1 (e.g. Ire1α) or Ire1 (e.g. Ire1α) pathway or phosphorylated Ire1 (e.g. Ire1α) pathway or pathway activated by Ire1 (e.g. Ire1α) phosphorylation). Thus, inhibition may include, at least in part, partially or totally decreasing stimulation, decreasing or reducing activation, or inactivating, desensitizing, or down-regulating signal transduction or enzymatic activity or the amount of a protein increased in a disease (e.g. level of Ire1 (e.g. Ire1α) activity or protein or level or activity of a component of an Ire1 (e.g. Ire1α) pathway or level of phosphorylated Ire1 (e.g. Ire1α) activity or protein or level or activity of a component of a phosphorylated Ire1 (e.g. Ire1α) pathway, wherein each is associated with cancer (e.g. multiple myeloma, or cancers of secretory cells), neurodegenerative diseases, demyelinating diseases, eye diseases, fibrotic diseases, or diabetes). Inhibition may include, at least in part, partially or totally decreasing stimulation, decreasing or reducing activation, or deactivating, desensitizing, or down-regulating signal transduction or enzymatic activity or the amount of a protein (e.g. Ire1 (e.g. Ire1α), phosphorylated Ire1 (e.g. Ire1α), protein downstream in a pathway from Ire1 (e.g. Ire1α), protein downstream in a pathway activated by phosphorylated Ire1 (e.g. Ire1α)) that may modulate the level of another protein or increase cell survival (e.g. decrease in phosphorylated Ire1 (e.g. Ire1α) pathway activity may increase cell survival in cells that may or may not have an increase in phosphorylated Ire1 (e.g. Ire1α) pathway activity relative to a non-disease control or decrease in Ire1 (e.g. Ire1α) pathway activity may increase cell survival in cells that may or may not have a increase in Ire1 (e.g. Ire1α) pathway activity relative to a non-disease control).

As defined herein, the term “activation”, “activate”, “activating” and the like in reference to a protein-activator (e.g. agonist) interaction means positively affecting (e.g. increasing) the activity or function of the protein (e.g. Ire1 (e.g. Ire1α), phosphorylated Ire1 (e.g. Ire1α), component of pathway including Ire1 (e.g. Ire1α), or component of pathway including phosphorylated Ire1 (e.g. Ire1α)) relative to the activity or function of the protein in the absence of the activator (e.g. compound described herein). In some embodiments, activation refers to an increase in the activity of a signal transduction pathway or signaling pathway (e.g. Ire1 (e.g. Ire1α) or phosphorylated Ire1 (e.g. Ire1α) pathway). Thus, activation may include, at least in part, partially or totally increasing stimulation, increasing or enabling activation, or activating, sensitizing, or up-regulating signal transduction or enzymatic activity or the amount of a protein decreased in a disease (e.g. level of Ire1 (e.g. Ire1α) activity or level of protein or activity decreased by phosphorylation of Ire1 (e.g. Ire1α) or protein associated with cancer (e.g. multiple myeloma, or cancers of secretory cells), neurodegenerative diseases, demyelinating diseases, eye diseases, fibrotic diseases, or diabetes). Activation may include, at least in part, partially or totally increasing stimulation, increasing or enabling activation, or activating, sensitizing, or up-regulating signal transduction or enzymatic activity or the amount of a protein (e.g. Ire1 (e.g. Ire1α), protein downstream of Ire1 (e.g. Ire1α), protein activated or upregulated by Ire1 (e.g. Ire1α), protein activated or upregulated by phosphorylation of Ire1 (e.g. Ire1α)) that may modulate the level of another protein or increase cell survival (e.g. increase in Ire1 (e.g. Ire1α) activity may increase cell survival in cells that may or may not have a reduction in Ire1 (e.g. Ire1α) activity relative to a non-disease control).

The term “modulator” refers to a composition that increases or decreases the level of a target molecule or the function of a target molecule. In some embodiments, a modulator of Ire1 (e.g. Ire1α) or Ire1 (e.g. Ire1α) pathway or phosphorylation of Ire1 (e.g. Ire1α) or pathway activated by phorphorylation of Ire1 (e.g. Ire1α) is a compound that reduces the severity of one or more symptoms of a disease associated with Ire1 (e.g. Ire1α) or Ire1 (e.g. Ire1α) pathway (e.g. disease associated with an increase in the level of Ire1 (e.g. Ire1α) activity or protein or Ire1 (e.g. Ire1α) pathway activity or protein or Ire1 (e.g. Ire1α) phorphorylation or pathway activated by Ire1 (e.g. Ire1α) phosphorylation, for example cancer (e.g. multiple myeloma, or cancers of secretory cells), neurodegenerative diseases, demylelinating diseases, eye diseases, fibrotic diseases, or diabetes) or a disease that is not caused by Ire1 (e.g. Ire1α) or Ire1 (e.g. Ire1α) pathway but may benefit from modulation of Ire1 (e.g. Ire1α) or Ire1 (e.g. Ire1α) pathway activity (e.g. decreasing in level or level of activity of Ire1 (e.g. Ire1α) or Ire1 (e.g. Ire1α) pathway). In embodiments, a modulator of Ire1 (e.g. Ire1α) or Ire1 (e.g. Ire1α) pathway (e.g. phosphorylated Ire1 (e.g. Ire1α) or phosphorylated Ire1 (e.g. Ire1α) pathway) is an anti-cancer agent. In embodiments, a modulator of Ire1 (e.g. Ire1α) or Ire1 (e.g. Ire1α) pathway (e.g. phosphorylated Ire1 (e.g. Ire1α) or phosphorylated Ire1 (e.g. Ire1α) pathway) is a neuroprotectant. In embodiments, a modulator of Ire1 (e.g. Ire1α) or Ire1 (e.g. Ire1α) pathway (e.g. phosphorylated Ire1 (e.g. Ire1α) or phosphorylated Ire1 (e.g. Ire1α) pathway) is an anti-demyelinating agent. In embodiments, a modulator of Ire1 (e.g. Ire1α) or Ire1 (e.g. Ire1α) pathway is a memory enhancing agent. In embodiments, a modulator of Ire1 (e.g. Ire1α) or Ire1 (e.g. Ire1α) pathway (e.g. phosphorylated Ire1 (e.g. Ire1α) or phosphorylated Ire1 (e.g. Ire1α) pathway) is an anti-diabetic agent. In embodiments, a modulator of Ire1 (e.g. Ire1α) or Ire1 (e.g. Ire1α) pathway (e.g. phosphorylated Ire1 (e.g. Ire1α) or phosphorylated Ire1 (e.g. Ire1α) pathway) is an anti-eye disease agent. In embodiments, a modulator of Ire1 (e.g. Ire1α) or Ire1 (e.g. Ire1α) pathway (e.g. phosphorylated Ire1 (e.g. Ire1α) or phosphorylated Ire1 (e.g. Ire1α) pathway) is an anti-fibrosis agent.

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a test animal. In some embodiments, a patient is a newborn animal. In some embodiments, a patient is a newborn human. In some embodiments, a patient is a juvenile animal. In some embodiments, a patient is a juvenile human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

“Disease” or “condition” refer to a state of being or health status of a patient or subject capable of being treated with a compound, pharmaceutical composition, or method provided herein. In some embodiments, the disease is a disease related to (e.g. caused by) an increase in the level of Ire1 (e.g. Ire1α), Ire1 (e.g. Ire1α) phosphorylation, Ire1 (e.g. Ire1α) RNase activity, or Ire1 (e.g. Ire1α) pathway activity, or pathway activated by phosphorylation of Ire1 (e.g. Ire1α). In some embodiments, the disease is a disease related to (e.g. caused by) neurodegeneration. In some embodiments, the disease is a disease related to (e.g. caused by) neural cell death. In some embodiments, the disease is a disease related to (e.g. caused by) cell death. In some embodiments, the disease is a disease related to (e.g. caused by) pancreatic cell death. In some embodiments, the disease is a disease related to (e.g. caused by) insulin-producing cell death. In some embodiments, the disease is a disease related to (e.g. caused by) loss of myelin. In some embodiments, the disease is a disease related to (e.g. caused by) reduction in myelin. In some embodiments, the disease is a disease related to (e.g. caused by) an increase in the level of Ire1 (e.g. Ire1α) activity (e.g. RNase activity), Ire1 (e.g. Ire1α) phosphorylation, Ire1 (e.g. Ire1α) pathway activity, or phosphorylated Ire1 (e.g. Ire1α) pathway activity. In some embodiments, the disease is cancer (e.g. multiple myeloma or cancers of secretory cells). In some embodiments, the disease is a neurodegenerative disease. In some embodiments, the disease is a demyelinating disease. In some embodiments, the disease is diabetes. In some embodiments, the disease is an interstitial lung disease (ILD). In some embodiments, the disease is idiopathic pulmonary fibrosis (IPF). In some embodiments, the disease is a fibrotic disease. In some embodiments, the disease is an eye disease (e.g., disease causing vision impairment).

Examples of diseases, disorders, or conditions include, but are not limited to, cancer (e.g. multiple myeloma or cancers of secretory cells), neurodegenerative diseases, demyelinating diseases, eye diseases, fibrotic diseases, and diabetes. In some instances, “disease” or “condition” refers to cancer. In some further instances, “cancer” refers to human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma.

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include multiple myeloma, blood cancers, lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g. hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, or melanoma. Additional examples include, cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the term “neurodegenerative disease” refers to a disease or condition in which the function of a subject's nervous system becomes impaired (e.g. relative to a control subject who does not have the neurodegenerative disease). Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Wolfram Syndrome, transverse myelitis, Charcot-Marie-Tooth (CMT) disease, or Tabes dorsalis. Examples of neurodegenerative diseases that may be treated with a compound, pharmaceutical composition, or method described herein include retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru.

As used herein, the term “demyelinating disease” refers to a disease or condition is which the myelin sheath of a subject's neurons is or becomes impaired (e.g. relative to a control subject who does not have the demyelinating disease). Examples of demyelinating disease that may be treated with a compound, pharmaceutical composition, or method described herein include Wolfram Syndrome, Pelizaeus-Merzbacher Disease, Transverse Myelitis, Charcot-Marie-Tooth Disease, and Multiple Sclerosis.

As used herein, the term “diabetes” or “diabetes mellitus” refers to a disease or condition is which a subject has high blood sugar. Examples of diabetes that may be treated with a compound, pharmaceutical composition, or method described herein include type I diabetes (type I diabetes mellitus), which is characterized by the subject's failure to produce insulin or failure to produce sufficient insulin for the subject's metabolic needs; type II diabetes (type II diabetes mellitus), which is characterized by insulin resistance (i.e. the failure of the subject (e.g. subject's cells) to use insulin properly; and gestational diabetes, which is high blood sugar during pregnancy. In embodiments, diabetes is type I diabetes. In embodiments, diabetes is type II diabetes. In embodiments, diabetes is gestational diabetes. In embodiments, diabetes is a disease or condition in which a subject has high blood sugar as determined by an A1C test (e.g. 6.5% or greater), fasting plasma glucose test (e.g. 126 mg/dL or greater), or oral glucose tolerance test (e.g. 200 mg/dL or greater). In embodiments, the diabetes is associated with Wolfram Syndrome.

As used herein, the term “eye disease” or “disease causing vision impairment” refers to a disease or condition is which the function of a subject's eye or eyes is impaired (e.g. relative to a subject without the disease). Examples of eye diseases that may be treated with a compound, pharmaceutical composition, or method described herein include retinitis pigmentosa, retinal degeneration, macular degeneration, and Wolfram Syndrome.

As used herein, the term “fibrosis” refers to the formation of excess fibrous connective tissue. The term “fibrotic disease” refers to a disease or condition caused by aberrant fibrosis or a disease or condition in which a symptom is aberrant fibrosis (e.g. relative to a control subject without the disease). Examples of fibrotic diseases that may be treated with a compound, pharmaceutical composition, or method described herein include idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), and hepatic fibrosis.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, mannitol, gum acacia, calcium phosphate, alginates, tragacanth, calcium silicate, microcrystalline cellulose, cellulose, syrup, and methyl cellulose, colors, and the like. The formulations can additionally include: lubricating agents such as talc, magnesium stearate, and mineral oil; wetting agents; emulsifying and suspending agents; preserving agents such as methyl and propylhydroxy benzoates; sweetening agents; and flavoring agents. The compositions described herein can be formulated so as to provide quick, sustained or delayed release of the active ingredient after administration to the patient by employing procedures known in the art. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means administration by any route, including systemic, local, oral administration, administration as a suppository, topical contact, intravenous, parenteral, intraperitoneal, intramuscular, intralesional, intrathecal, intracranial, intranasal or subcutaneous administration, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), transmucosal (e.g., buccal, sublingual, palatal, gingival, nasal, vaginal, rectal, or transdermal), ocular, or the implantation of a slow-release device, e.g., a mini-osmotic pump, to a subject. Parenteral administration includes, e.g., intravenous, intraarterial, intramuscular, intra-arteriole, intradermal, subcutaneous, intraperitoneal, intrathecal, intraventricular, and intracranial. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Methods for ocular delivery can include topical administration (eye drops), subconjunctival, periocular or intravitreal injection or introduction by balloon catheter or ophthalmic inserts surgically placed in the conjunctival sac. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, treatment for an eye disease, treatment for fibrosis, treatment for a demyelinating disease, diabetes treatment, or treatment for a neurodegenerative disease). The compound of the invention can be administered alone or can be coadministered to the patient. The compositions (e.g. compounds) described herein can also be formulated in combination with one or more additional active ingredients which can include any pharmaceutical agent such as anti viral agents, vaccines, antibodies, immune enhancers, immune suppressants, anti inflammatory agents and the like. Coadministration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. Ire1 (e.g. Ire1α) or component of Ire1 (e.g. Ire1α) signal transduction pathway or component of phosphorylated Ire1 (e.g. Ire1α) pathway), and/or reducing, eliminating, or slowing the progression of disease symptoms (e.g. symptoms of cancer (e.g. multiple myeloma or cancers of secretory cells), neurodegenerative diseases, demyelinating diseases, eye diseases, fibrotic diseases, or diabetes). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer (e.g. multiple myeloma or cancers of secretory cells), neurodegenerative diseases, demyelinating diseases, eye diseases, fibrotic diseases, or diabetes), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating cancer (e.g. multiple myeloma or cancers of secretory cells), neurodegenerative diseases, demyelinating diseases, eye diseases, fibrotic diseases, or diabetes, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for cancer (e.g. multiple myeloma or cancers of secretory cells), neurodegenerative diseases, demyelinating diseases, or diabetes, such as surgery.

The term “Ire1” or “Ire1α” or “ERN1” refers to the protein “Serine/threonine-protein kinase/endoribonuclease IRE1” a.k.a. “Endoplasmic reticulum to nucleus signaling 1”. In embodiments, “Ire1” or “Ire1α” or “ERN1” refers to the human protein. Included in the term “Ire1” or “Ire1α” or “ERN1” are the wildtype and mutant forms of the protein. In embodiments, “Ire1” or “Ire1α” or “ERN1” refers to the protein associated with Entrez Gene 2081, OMIM 604033, UniProt 075460, and/or RefSeq (protein) NM_001433. In embodiments, the reference numbers immediately above refer to the protein, and associated nucleic acids, known as of the date of filing of this application. In embodiments, “Ire1” or “Ire1α” or “ERN1” refers to the wildtype human protein. In embodiments, “Ire1” or “Ire1α” or “ERN1” refers to the wildtype human nucleic acid. In embodiments, “Ire1” or “Ire1α” or “ERN1” refers to the protein or nucleic acid corresponding to GI:153946420. In embodiments, “Ire1” or “Ire1α” or “ERN1” refers to the protein or nucleic acid corresponding to NM_001433.3 (SEQ ID NO:25). In embodiments, “Ire1” or “Ire1α” or “ERN1” refers to the protein or nucleic acid corresponding to GI:153946421. In embodiments, “Ire1” or “Ire1α” or “ERN1” refers to the protein or nucleic acid corresponding to NP_001424.3 (SEQ ID NO:26).

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK inhibitors, alkylating agents, anti-metabolites, plant alkaloids, topoisomerase inhibitors, antitumor antibiotics, platinum-based compounds, adrenocortical suppressants, epipodophyllotoxins, antibiotics, enzymes, inhibitors of mitogen-activated protein kinase signaling, antibodies, doxorubicin, vincristine, etoposide, gemcitabine, imatinib (Gleevec®), agents that arrest cells in the G2-M phases and/or modulate the formation or stability of microtubules (e.g. Taxol™ (i.e. paclitaxel), steroids, aromatase inhibitors, gonadotropin-releasing hormone agonists (GnRH), adrenocorticosteroids, progestins, estrogens, antiestrogens, androgens, antiandrogens, immunotoxins, radioimmunotherapy, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Gudrin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

“Anti-diabetic agent” or “antidiabetic agent” is used in accordance with its plain ordinary meaning and refers to a composition (e.g. compound, drug, antagonist, inhibitor, modulator) having the ability to lower blood glucose levels in a subject. In some embodiments, an anti-diabetic agent is an agent identified herein having utility in methods of treating diabetes. In some embodiments, an anti-diabetic agent is an agent approved by the FDA or similar regulatory agency of a country other than the USA, for treating diabetes. Examples of anti-diabetic agents include, but are not limited to, insulin, insulin sensitizers (e.g. biguanides (e.g. metformin, phenformin, or buformin), thiazolidinediones (e.g. rosiglitazone, pioglitazone, troglitazone)), secretagogues (e.g. sulfonylureas (e.g. tolbutamide, acetohexamide, tolazamide, chlorpropamide, glipizide, glyburide, glibenclamide, glimepiride, gliclazide, glycopyramide, gliquidone), meglitinides (e.g. repaglinide, nateglinide)), alpha-glucosidase inhibitors (e.g. miglitol, acarbose, voglibose), peptide analog antidiabetic agents (e.g. incretins (glucagon-like peptide-1, gastric inhibitory peptide), glucagon-like peptide agonists (e.g. exenatide, liraglutide, taspoglutide), gastric inhibitoty peptide analogs, or dipeptidyl peptidase-4 inhibitors (e.g. vildagliptin, sitagliptin, saxagliptin, linagliptin, allogliptin, septagliptin), amylin agonist analogues (e.g. pramlintide).

“Agent for reducing Ire1 RNase activity” or “Ire1 RNase inhibitor” or “Ire1 endoribonuclease inhibitor” are equivalent and are used in accordance with their plain ordinary meaning and refer to a composition (e.g. compound, drug, antagonist, inhibitor, modulator, Ire1 (e.g., Ire1α) ribonuclease modulating compound) having the ability to lower the ribonuclease activity of Ire1. In some embodiments, an agent for reducing Ire1 RNase activity is an agent approved by the FDA or similar regulatory agency of a country other than the USA, for reducing the ribonuclease activity of Ire1 (e.g., Ire1α, human Ire1, human Ire1α). In some embodiments, an agent for reducing Ire1 (e.g. Ire1α) RNase activity is an agent approved by the FDA or similar regulatory agency of a country other than the USA, for treating a disease mediated by (e.g., caused by or associated with) the ribonuclease activity of Ire1 (e.g., Ire1α, human Ire1, human Ire1α). In embodiments an agent for reducing Ire1 (e.g. Ire1α) RNase activity is an agent that inhibits a pathway activated by Ire1 (e.g. Ire1α) RNase activity. In embodiments, an agent for reducing Ire1 (e.g. Ire1α) RNase activity is STF-083010. In embodiments, an agent for reducing Ire1 (e.g. Ire1α) RNase activity is a derivative of STF-083010. In embodiments, an agent for reducing Ire1 (e.g. Ire1α) RNase activity is MKC-3946. In embodiments, an agent for reducing Ire1 (e.g. Ire1α) RNase activity is a derivative of MKC-3946. In embodiments, an agent for reducing Ire1 (e.g. Ire1α) RNase activity is 4μ8C. In embodiments, an agent for reducing Ire1 (e.g. Ire1α) RNase activity is a derivative of 4μ8C. In embodiments, an agent for reducing Ire1 (e.g. Ire1α) RNase activity is a salicylaldehyde. In embodiments, an agent for reducing Ire1 (e.g. Ire1α) RNase activity is 3-methoxy-6-bromosalicylaldehyde. In embodiments, an agent for reducing Ire1 (e.g. Ire1α) RNase activity is a derivative of 3-methoxy-6-bromosalicylaldehyde. In embodiments, an agent for reducing Ire1 (e.g. Ire1α) RNase activity is toyocamycin. In embodiments, an agent for reducing Ire1 (e.g. Ire1α) RNase activity is a prodrug of a compound described above. In embodiments, an agent for reducing Ire1 (e.g. Ire1α) RNase activity is an analog of a compound described above. In embodiments, an agent for reducing Ire1 (e.g. Ire1α) RNase activity is a derivative of toyocamycin. In embodiments, an agent for reducing Ire1 (e.g. Ire1α) RNase activity is a compound described in WO2012064774, US20130303599, 61/412,207, WO2011127070, US20130116247, WO2007101224, WO2008154484, or 7,858,666. The entire contents of these patents are incorporated herein by reference in their entirety for all purposes.

“Synergy,” “synergistic” and the like when referring to a combined effect of a plurality of compounds (e.g., modulators, inhibitors, kinase inhibitor, ribonuclease inhibitor, Ire1 inhibitor, Ire1 kinase inhibitor, Ire1 ribonuclease inhibitor, Ire1 (e.g., Ire1α) ribonuclease modulating compound, Ire1 (e.g., Ire1α) kinase modulating compound) is used in accordance with its plain ordinary meaning and refers to an interaction of a plurality of compounds to produce an effect greater than the sum of their individual effects. Synergy between a Ire1 (e.g., Ire1α) kinase modulating compound and Ire1 (e.g., Ire1α) ribonuclease modulating compound on the activity of Ire1 (e.g., Ire1 RNase activity) results in a greater than additive modulation (e.g., reduction) in Ire1 activity (e.g., Ire1 RNase activity). Synergy between a kinase inhibitor and ribonuclease inhibitor on the activity of Ire1 (e.g., Ire1 RNase activity) results in a greater than additive reduction in Ire1 activity. In embodiments, synergy of inhibition between a plurality of compounds may result in about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater inhibition of Ire1 activity than the sum of the inhibition of the plurality of compounds when used individually and separately. In embodiments, synergy of inhibition between a plurality of compounds may result in 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater inhibition of Ire1 activity than the sum of the inhibition of the plurality of compounds when used individually and separately. In embodiments, synergy of inhibition between a plurality of compounds may result in about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9 fold inhibition of Ire1 activity compared to the sum of the inhibition of the plurality of compounds when used individually and separately. In embodiments, synergy of inhibition between a plurality of compounds may result in 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9 fold inhibition of Ire1 activity compared to the sum of the inhibition of the plurality of compounds when used individually and separately.

The term “synergistic amount” is used in accordance with its plain ordinary meaning and refers to an amount of a composition (e.g. compound, drug, antagonist, inhibitor, modulator) when the composition is a component in a synergistic combination of compositions. The synergistic amount is an amount of the component composition that is reduced compared to the amount to the composition when not a component of a synergistic combination of compositions (i.e., when the composition is used separately from the other active components of the synergistic combination of compositions). The synergistic amount of the composition produces an effect (e.g., inhibition of Ire1 activity, inhibition of Ire1 RNase activity) equal to the effect of a larger amount of the composition when used separately from the other active components of the synergistic combination of compositions. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the component composition when used separately from the other active components of the synergistic combination of compositions. In embodiments, a synergistic amount may be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the component composition when used separately from the other active components of the synergistic combination of compositions.

A “combined synergistic amount” as used here refers to the sum of a first amount and a second amount that results in a synergistic effect (i.e. an effect greater than an additive effect). The synergistic effect may be an Ire1 activity decreasing effect. The synergistic effect may be an Ire1 ribonuclease activity decreasing effect. In vivo, the synergistic effect may be a disease-treating effect such as for a neurodegenerative disease (i.e. a neurodegenerative disease-treating synergistic effect), demyelinating disease (i.e. a demyelinating disease-treating synergistic effect), cancer (i.e. an anti-cancer effect), or diabetes (i.e. a diabetes-treating synergistic effect). Other diseases include retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, Kuru, Wolfram Syndrome, Pelizaeus-Merzbacher Disease, Transverse Myelitis, Charcot-Marie-Tooth Disease, Multiple Sclerosis, multiple myeloma, type I diabetes, type II diabetes, idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

The term “Ire1 kinase modulating compound” is used in accordance with its plain ordinary meaning and refers to a compound (e.g., small molecule compound) capable of modulating the kinase activity of Ire1 (e.g., Ire1α). In embodiments, an Ire1 kinase modulating compound is an Ire1 kinase inhibiting compound. In embodiments, an Ire1 kinase modulating compound binds the Ire1 kinase domain. In embodiments, an Ire1 kinase modulating compound does not bind the Ire1 ribonuclease domain. In embodiments, an Ire1 kinase modulating compound does not directly modulate (e.g., inhibit) the Ire1 ribonuclease activity. In embodiments, an Ire1 kinase modulating compound is a compound as described herein, including in an aspect, embodiment, table, example, figure, or claim (e.g., compound of formula I and embodiments thereof).

The term “Ire1 ribonuclease modulating compound” is used in accordance with its plain ordinary meaning and refers to a compound (e.g., small molecule compound) capable of modulating the ribonuclease activity of Ire1 (e.g., Ire1α). In embodiments, an Ire1 ribonuclease modulating compound is an Ire1 ribonuclease inhibiting compound. In embodiments, an Ire1 ribonuclease modulating compound binds the Ire1 ribonuclease domain. In embodiments, an Ire1 ribonuclease modulating compound does not bind the Ire1 kinase domain. In embodiments, an Ire1 ribonuclease modulating compound does not directly modulate (e.g., inhibit) the Ire1 kinase activity. In embodiments, an Ire1 ribonuclease modulating compound is an “agent for reducing Ire1 (e.g. Ire1α) RNase activity” or “Ire1 RNase inhibitor” or “Ire1 endoribonuclease inhibitor”. An Ire1 ribonuclease modulating compound may be STF-083010, MKC-3946, 4μ8C, 3-methoxy-6-bromosalicylaldehyde, a salicylaldehyde, or toyocamycin. An Ire1 ribonuclease modulating compound may be STF-083010, MKC-3946, 4μ8C, 3-methoxy-6-bromosalicylaldehyde, a salicylaldehyde containing compound, toyocamycin or a 3-hydroxybenzaldehyde containing compound or a 4-hydroxybenzaldehyde containing compound.

The term “STF-083010” is used in accordance with its common meaning and refers to the Ire1 ribonuclease modulator commonly known by that name. The term “STF-083010” is N-[(2-Hydroxy-1-naphthalenyl)methylene]-2-thiophenesulfonamide. The term “MKC-3946” is used in accordance with its common meaning and refers to the Ire1 ribonuclease modulator commonly known by that name. The term “MKC-3946” is

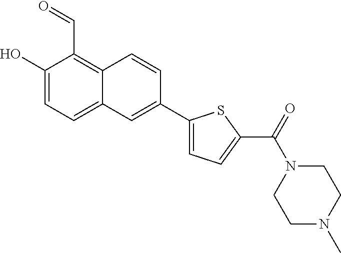 [SMILES: CN1CCN(C(=O)c2ccc(-c3ccc4c(C=O)c(O)ccc4c3)s2)CC1]

(2-hydroxy-6-(5-(4-methylpiperazine-1-carbonyl)thiophen-2-yl)-1-naphthaldehyde). The term “4μ8C” is used in accordance with its common meaning and refers to the Ire1 ribonuclease modulator commonly known by that name. The term “4μ8C” is 7-Hydroxy-4-methyl-2-oxo-2H-1-benzopyran-8-carboxaldehyde. The term “a salicylaldehyde” is used in accordance with its common meaning and refers to compounds including the salicylaldehyde chemical moiety. The term “a salicylaldehyde” is a compound including a 2-hydroxybenzaldehyde moiety. The term “toyocamycin” is used in accordance with its common meaning and refers to the Ire1 ribonuclease modulator commonly known by that name. The term “toyocamycin” is 4-Aminopyrrolo[2,3-d] pyrimidine-5-carbonitrile 7-(β-D-ribofuranoside), 7-Deaza-7-cyanoadenosine or 4-Amino-7-β-D-ribofuranosyl-7H-pyrrolo[2,3-d]pyrimidine-5-carbonitrile.

### B. COMPOSITIONS

In an aspect is provided a compound, or a pharmaceutically acceptable salt thereof, having the formula:

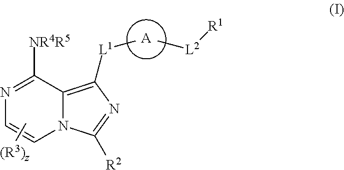

wherein, ring A is substituted or unsubstituted cycloalkylene, substituted or unsubstituted heterocycloalkylene, substituted or unsubstituted arylene, or substituted or unsubstituted heteroarylene; L¹ is a bond or unsubstituted C₁-C₅ alkylene; L² is a bond, —NR^{6a}—, —O—, —S—, —C(O)—, —S(O)—, —S(O)₂—, —NR^{6a}C(O)—, —C(O)(CH₂)_{z2}—, —C(O)NR^{6b}—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹ is hydrogen, oxo, halogen, —CX₃, —CN, —SO₂Cl, —SO₁R¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, oxo, halogen, —CX^{b}₃, —CN, —SO₂Cl, —SOₙ₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, —NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ and R⁵ are independently hydrogen or unsubstituted C₁-C₆ alkyl; R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl; each occurrence of the symbols n, n1, and n2 is independently an integer from 0 to 4; each occurrence of the symbols m, m1, m2, v, v1, and v2 is independently an integer from 1 to 2; the symbol z is an integer from 0 to 2; the symbol z2 is an integer from 1 to 4; and each occurrence of the symbols X, X^{a}, and X^{b} is independently a halogen.

In embodiments, ring A is substituted or unsubstituted monocyclic cycloalkylene, substituted or unsubstituted monocyclic heterocycloalkylene, substituted or unsubstituted monocyclic arylene, or substituted or unsubstituted monocyclic heteroarylene. In embodiments, ring A is substituted monocyclic cycloalkylene, substituted monocyclic heterocycloalkylene, substituted monocyclic arylene, or substituted monocyclic heteroarylene. In embodiments, ring A is unsubstituted monocyclic cycloalkylene, unsubstituted monocyclic heterocycloalkylene, unsubstituted monocyclic arylene, or unsubstituted monocyclic heteroarylene.

In embodiments, ring A is substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, ring A is substituted C₃-C₈ cycloalkylene, substituted 3 to 8 membered heterocycloalkylene, substituted C₆-C₁₀ arylene, or substituted 5 to 10 membered heteroarylene. In embodiments, ring A is unsubstituted C₃-C₈ cycloalkylene, unsubstituted 3 to 8 membered heterocycloalkylene, unsubstituted C₆-C₁₀ arylene, or unsubstituted 5 to 10 membered heteroarylene. In embodiments, ring A is substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 9 membered heteroarylene. In embodiments, ring A is substituted C₃-C₆ cycloalkylene, substituted 3 to 6 membered heterocycloalkylene, substituted C₆-C₁₀ arylene, or substituted 5 to 9 membered heteroarylene. In embodiments, ring A is unsubstituted C₃-C₆ cycloalkylene, unsubstituted 3 to 6 membered heterocycloalkylene, unsubstituted C₆-C₁₀ arylene, or unsubstituted 5 to 9 membered heteroarylene.

In embodiments, ring A is substituted or unsubstituted arylene or substituted or unsubstituted heteroarylene. In embodiments, ring A is substituted or unsubstituted C₆-C₁₀ arylene. In embodiments, ring A is unsubstituted naphthalenyl (i.e. divalent naphthalene moiety). In embodiments, ring A is substituted naphthalenyl. In embodiments, ring A is unsubstituted phenylene (divalent benzene moiety or benzene-di-yl). In embodiments, ring A is substituted phenylene (divalent benzene moiety or benzene-di-yl).

In embodiments, ring A is R⁴¹-substituted or unsubstituted cycloalkylene, R⁴¹-substituted or unsubstituted heterocycloalkylene, R⁴¹-substituted or unsubstituted arylene, or R⁴¹-substituted or unsubstituted heteroarylene. In embodiments, ring A is substituted with 1 to 6 optionally different R⁴¹ substituents. In embodiments, ring A is substituted with 1 R⁴¹ substituent. In embodiments, ring A is substituted with 2 optionally different R⁴¹ substituents. In embodiments, ring A is substituted with 3 optionally different R⁴¹ substituents. In embodiments, ring A is substituted with 4 optionally different R⁴¹ substituents. In embodiments, ring A is substituted with 5 optionally different R⁴¹ substituents. In embodiments, ring A is substituted with 6 optionally different R⁴¹ substituents.

R⁴¹ is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴²-substituted or unsubstituted alkyl, R⁴²-substituted or unsubstituted heteroalkyl, R⁴²-substituted or unsubstituted cycloalkyl, R⁴²-substituted or unsubstituted heterocycloalkyl, R⁴²-substituted or unsubstituted aryl, or R⁴²-substituted or unsubstituted heteroaryl. In embodiments, R⁴¹ is —OCH₃.

R⁴² is independently oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴³-substituted or unsubstituted alkyl, R⁴³-substituted or unsubstituted heteroalkyl, R⁴³-substituted or unsubstituted cycloalkyl, R⁴³-substituted or unsubstituted heterocycloalkyl, R⁴³-substituted or unsubstituted aryl, or R⁴³-substituted or unsubstituted heteroaryl.

In embodiments, R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)m, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, or —OCHX₂.

In embodiments, R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹ is hydrogen, substituted alkyl, substituted heteroalkyl, substituted cycloalkyl, substituted heterocycloalkyl, substituted aryl, or substituted heteroaryl. In embodiments, R¹ is hydrogen, unsubstituted alkyl, unsubstituted heteroalkyl, unsubstituted cycloalkyl, unsubstituted heterocycloalkyl, unsubstituted aryl, or unsubstituted heteroaryl. In embodiments, R¹ is hydrogen. In embodiments, R¹ is hydrogen and L² is —NHC(O)—.

In embodiments, R¹ is hydrogen, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R¹ is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 9 membered heteroaryl.

In embodiments, R¹ is substituted or unsubstituted cycloalkyl, substituted or unsubstituted heterocycloalkyl, substituted or unsubstituted aryl, or substituted or unsubstituted heteroaryl. In embodiments, R¹ is substituted or unsubstituted aryl or substituted or unsubstituted heteroaryl. In embodiments, R¹ is substituted phenyl. In embodiments, R¹ is unsubstituted phenyl. In embodiments, R¹ is phenyl substituted with —CF₃ or halogen. In embodiments, R¹ is phenyl meta-substituted with —CF₃. In embodiments, R¹ is phenyl meta-substituted with —F. In embodiments, R¹ is phenyl meta-substituted with —Cl. In embodiments, R¹ is phenyl meta-substituted with —Br. In embodiments, R¹ is phenyl meta-substituted with —I. In embodiments, R¹ is phenyl meta-substituted with —CH₃. In embodiments, R¹ is —OPh, —CH₂Ph, —OCH₂Ph, —NHC(O)H, or —CHO. In embodiments, R¹ is phenyl meta-substituted with —CCl₃. In embodiments, R¹ is phenyl para-substituted with —CF₃, —Cl, —OCF₃, —CH₃, —F, —OCH₃, —OPh, —CH₂Ph, or —CHO. In embodiments, R¹ is phenyl meta-substituted with —CF₃, —Cl, —OCF₃, —CH₃, —F, —OCH₃, —OPh, —CH₂Ph, or —CHO. In embodiments, R¹ is phenyl ortho-substituted with —CF₃, —Cl, —OCF₃, —CH₃, —F, —OCH₃, —OPh, —CH₂Ph, or —CHO. In embodiments, R¹ is aryl meta-substituted with —CF₃, —Cl, —OCF₃, —CH₃, —F, —OCH₃, —OPh, —CH₂Ph, or —CHO. In embodiments, R¹ is aryl ortho-substituted with —CF₃, —Cl, —OCF₃, —CH₃, —F, —OCH₃, —OPh, —CH₂Ph, or —CHO. In embodiments, R¹ is aryl para-substituted with —CF₃, —Cl, —OCF₃, —CH₃, —F, —OCH₃, —OPh, —CH₂Ph, or —CHO. In embodiments, R¹ is aryl substituted with —CF₃, —Cl, —OCF₃, —CH₃, —F, —OCH₃, —OPh, —CH₂Ph, or —CHO. In embodiments, R¹ is heteroaryl substituted with —CF₃, —Cl, —OCF₃, —CH₃, —F, —OCH₃, —OPh, —CH₂Ph, or —CHO. In embodiments, R¹ is phenyl substituted with —CF₃, —Cl, —OCF₃, —CH₃, —F, —OCH₃, —OPh, —CH₂Ph, or —CHO. In embodiments, R¹ is 5 to 6 membered heteroaryl substituted with —CF₃, —Cl, —OCF₃, —CH₃, —F, —OCH₃, —OPh, —CH₂Ph, or —CHO. In embodiments, R¹ is unsubstituted cyclohexyl. In embodiments, R¹ is substituted cyclohexyl. In embodiments, R¹ is unsubstituted cyclopenyl. In embodiments, R¹ is substituted cyclopenyl. In embodiments, R¹ is unsubstituted cyclobutyl. In embodiments, R¹ is substituted cyclobutyl. In embodiments, R¹ is unsubstituted cyclopropyl. In embodiments, R¹ is substituted cyclopropyl.

In embodiments, R¹ is a substituted or unsubstituted heteroaryl selected from the group consisting of pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, and quinolyl.

In some embodiments of the compounds provided herein, R¹ is independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In embodiments, R¹ is substituted with 1 to 6 optionally different R¹¹ substituents. In embodiments, R¹ is substituted with 1 R¹¹ substituent. In embodiments, R¹ is substituted with 2 optionally different R¹¹ substituents. In embodiments, R¹ is substituted with 3 optionally different R¹¹ substituents. In embodiments, R¹ is substituted with 4 optionally different R¹¹ substituents. In embodiments, R¹ is substituted with 5 optionally different R¹¹ substituents. In embodiments, R¹ is substituted with 6 optionally different R¹¹ substituents. In embodiments, R¹ is substituted with 7 optionally different R¹¹ substituents. In embodiments, R¹ is phenyl substituted with 1 to 5 optionally different R¹¹ substituents. In embodiments, R¹ is phenyl substituted with 1 R¹¹ substituent. In embodiments, R¹ is phenyl substituted with 2 optionally different R¹¹ substituents. In embodiments, R¹ is phenyl substituted with 3 optionally different R¹¹ substituents. In embodiments, R¹ is phenyl substituted with 4 optionally different R¹¹ substituents. In embodiments, R¹ is phenyl substituted with 5 optionally different R¹¹ substituents. In embodiments, R¹ is aryl substituted with 1 to 6 optionally different R¹¹ substituents. In embodiments, R¹ is aryl substituted with 1 R¹¹ substituent. In embodiments, R¹ is aryl substituted with 2 optionally different R¹¹ substituents. In embodiments, R¹ is aryl substituted with 3 optionally different R¹¹ substituents. In embodiments, R¹ is aryl substituted with 4 optionally different R¹¹ substituents. In embodiments, R¹ is aryl substituted with 5 optionally different R¹¹ substituents. In embodiments, R¹ is aryl substituted with 6 optionally different R¹¹ substituents. In embodiments, R¹ is aryl substituted with 7 optionally different R substituents. In embodiments, R¹ is heteroaryl substituted with 1 to 6 optionally different R″ substituents. In embodiments, R¹ is heteroaryl substituted with 1 R¹¹ substituent. In embodiments, R¹ is heteroaryl substituted with 2 optionally different R¹¹ substituents. In embodiments, R¹ is heteroaryl substituted with 3 optionally different R¹¹ substituents. In embodiments, R¹ is heteroaryl substituted with 4 optionally different R¹¹ substituents. In embodiments, R¹ is heteroaryl substituted with 5 optionally different R¹¹ substituents. In embodiments, R¹ is heteroaryl substituted with 6 optionally different R¹¹ substituents. In embodiments, R¹ is heteroaryl substituted with 7 optionally different R¹¹ substituents.

R¹¹ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl. In embodiments, R¹¹ is —CCl₃, —CF₃, —Cl, —OCF₃, —CH₃, —F, —OCH₃, —OPh, —CH₂Ph, or —CHO.

R¹² is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or R¹³-substituted or unsubstituted heteroaryl.

In embodiments, R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is hydrogen, substituted alkyl, substituted heteroalkyl, substituted cycloalkyl, substituted heterocycloalkyl, substituted aryl, or substituted heteroaryl. In embodiments, R² is hydrogen, unsubstituted alkyl, unsubstituted heteroalkyl, unsubstituted cycloalkyl, unsubstituted heterocycloalkyl, unsubstituted aryl, or unsubstituted heteroaryl.

In embodiments, R² is hydrogen, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R² is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 9 membered heteroaryl.

In embodiments, R² is substituted or unsubstituted alkyl. In embodiments, R² is substituted or unsubstituted C₁-C₆ alkyl. In embodiments, R² is unsubstituted C₁-C₆ alkyl. In embodiments, R² is unsubstituted methyl. In embodiments, R² is unsubstituted isopropyl. In embodiments, R² is unsubstituted ethyl. In embodiments, R² is unsubstituted propyl (e.g. n-propyl or isopropyl). In embodiments, R² is unsubstituted isopropyl. In embodiments, R² is unsubstituted butyl (e.g. n-butyl, sec-butyl, isobutyl, or tert-butyl). In embodiments, R² is unsubstituted tert-butyl. In embodiments, R² is unsubstituted iso-butyl. In embodiments, R² is unsubstituted pentyl (e.g. n-pentyl, tert-pentyl, neopentyl, isopentyl, sec-pentyl, or 3-pentyl). In embodiments, R² is unsubstituted cyclopropyl. In embodiments, R² is unsubstituted cyclobutyl. In embodiments, R² is unsubstituted cyclopentyl. In embodiments, R² is unsubstituted cyclohexyl.

In some embodiments, R² is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁴-substituted or unsubstituted alkyl, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or R¹⁴-substituted or unsubstituted heteroaryl.

R¹⁴ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁵-substituted or unsubstituted alkyl, R¹⁵-substituted or unsubstituted heteroalkyl, R¹⁵-substituted or unsubstituted cycloalkyl, R¹⁵-substituted or unsubstituted heterocycloalkyl, R¹⁵-substituted or unsubstituted aryl, or R¹⁵-substituted or unsubstituted heteroaryl.

R¹⁵ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl.

In embodiments, R³ is independently hydrogen, oxo, halogen, —CX^{b}₃, —CN, —SOₙ₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, —NR^{7b}SOₙ₂R^{10b}, —NR^{7b}C═(O)R^{9b}, —NR^{7b}C(O)OR^{9b}, —NR^{7b}OR^{9b}, —OCX^{b}₃, or —OCHX^{b}₂. In embodiments, R³ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen, substituted alkyl, substituted heteroalkyl, substituted cycloalkyl, substituted heterocycloalkyl, substituted aryl, or substituted heteroaryl. In embodiments, R³ is independently hydrogen, unsubstituted alkyl, unsubstituted heteroalkyl, unsubstituted cycloalkyl, unsubstituted heterocycloalkyl, unsubstituted aryl, or unsubstituted heteroaryl.

In embodiments, R³ is independently hydrogen, substituted or unsubstituted C₁-C₈ alkyl, substituted or unsubstituted 2 to 8 membered heteroalkyl, substituted or unsubstituted C₃-C₅ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R³ is independently hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 9 membered heteroaryl.

In embodiments, R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is independently hydrogen. In embodiments, R³ is independently halogen.

In some embodiments, R³ is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl.

R¹⁷ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁸-substituted or unsubstituted alkyl, R¹⁸-substituted or unsubstituted heteroalkyl, R¹⁸-substituted or unsubstituted cycloalkyl, R¹⁸-substituted or unsubstituted heterocycloalkyl, R¹⁸-substituted or unsubstituted aryl, or R¹⁸-substituted or unsubstituted heteroaryl.

R¹⁸ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl.

In embodiments, R⁴ and R⁵ are independently hydrogen. In embodiments, R⁴ and R⁵ are independently unsubstituted C₁-C₆ alkyl. In embodiments, R⁴ and R⁵ are independently unsubstituted C₁-C₅ alkyl. In embodiments, R⁴ and R⁵ are independently unsubstituted C₁-C₄ alkyl. In embodiments, R⁴ and R⁵ are independently unsubstituted C₁-C₃ alkyl. In embodiments, R⁴ and R⁵ are independently unsubstituted C₁-C₂ alkyl. In embodiments, R⁴ and R⁵ are independently unsubstituted methyl.

In embodiments, L¹ is a bond. In embodiments, L¹ is unsubstituted C₁-C₅ alkylene. In embodiments, L¹ is unsubstituted C₁-C₄ alkylene. In embodiments, L¹ is unsubstituted C₁-C₃ alkylene. In embodiments, L¹ is unsubstituted C₁-C₂ alkylene. In embodiments, L¹ is unsubstituted methylene.

In embodiments, L² is a bond. In embodiments, L² is —NR^{6a}—. In embodiments, L² is —O—. In embodiments, L² is —S—. In embodiments, L² is —C(O)—. In embodiments, L² is —S(O)—. In embodiments, L² is —S(O)₂—. In embodiments, L² is —C(O)(CH₂)_{z2}—. In embodiments, L² is —NR^{6a}C(O)—. In embodiments, L² is —C(O)NR^{6b}—. In embodiments, L² is —NR^{6a}C(O)O—. In embodiments, L² is —NR^{6a}C(O)NR^{6b}—. In embodiments, L² is —NH—. In embodiments, L² is —NHC(O)—. In embodiments, L² is —C(O)NH—. In embodiments, L² is —NHC(O)NH—. In embodiments, L² is —NHC(O)OCH₂—. In embodiments, L² is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is —C(O)(CH₂)—. In embodiments, L² is —C(O)(CH₂)₂—. In embodiments, L² is —C(O)(CH₂)₃—. In embodiments, L² is —C(O)(CH₂)₄—.

In embodiments, L² is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L² is substituted alkylene, substituted heteroalkylene, substituted cycloalkylene, substituted heterocycloalkylene, substituted arylene, or substituted heteroarylene. In embodiments, L² is unsubstituted alkylene, unsubstituted heteroalkylene, unsubstituted cycloalkylene, unsubstituted heterocycloalkylene, unsubstituted arylene, or unsubstituted heteroarylene.

In embodiments, L² is substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L² is substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 9 membered heteroarylene.

In some embodiments, L² is independently R⁴⁴-substituted or unsubstituted alkylene, R⁴⁴-substituted or unsubstituted heteroalkylene, R⁴⁴-substituted or unsubstituted cycloalkylene, R⁴⁴-substituted or unsubstituted heterocycloalkylene, R⁴⁴-substituted or unsubstituted arylene, or R⁴⁴-substituted or unsubstituted heteroarylene.

R⁴⁴ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴⁵-substituted or unsubstituted alkyl, R⁴⁵-substituted or unsubstituted heteroalkyl, R⁴⁵-substituted or unsubstituted cycloalkyl, R⁴⁵-substituted or unsubstituted heterocycloalkyl, R⁴⁵-substituted or unsubstituted aryl, or R⁴⁵-substituted or unsubstituted heteroaryl.

R⁴⁵ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴⁶-substituted or unsubstituted alkyl, R⁴⁶-substituted or unsubstituted heteroalkyl, R⁴⁶-substituted or unsubstituted cycloalkyl, R⁴⁶-substituted or unsubstituted heterocycloalkyl, R⁴⁶-substituted or unsubstituted aryl, or R⁴⁶-substituted or unsubstituted heteroaryl.

In embodiments, R^{6a} is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{6a} is hydrogen, substituted alkyl, substituted heteroalkyl, substituted cycloalkyl, substituted heterocycloalkyl, substituted aryl, or substituted heteroaryl. In embodiments, R^{6a} is hydrogen, unsubstituted alkyl, unsubstituted heteroalkyl, unsubstituted cycloalkyl, unsubstituted heterocycloalkyl, unsubstituted aryl, or unsubstituted heteroaryl. In embodiments, R^{6a} is hydrogen. In embodiments, R^{6a} is unsubstituted methyl. In embodiments, R^{6a} is unsubstituted ethyl. In embodiments, R^{6a} is unsubstituted propyl.

In embodiments, R^{6a} is hydrogen, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R^{6a} is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 9 membered heteroaryl.

In some embodiments of the compounds provided herein, R^{6a} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{26a}-substituted or unsubstituted alkyl, R^{26a}-substituted or unsubstituted heteroalkyl, R^{26a}-substituted or unsubstituted cycloalkyl, R^{26a}-substituted or unsubstituted heterocycloalkyl, R^{26a}-substituted or unsubstituted aryl, or R^{26a}-substituted or unsubstituted heteroaryl.

R^{26a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{27a}-substituted or unsubstituted alkyl, R^{27a}-substituted or unsubstituted heteroalkyl, R^{27a}-substituted or unsubstituted cycloalkyl, R^{27a}-substituted or unsubstituted heterocycloalkyl, R^{27a}-substituted or unsubstituted aryl, or R^{27a}-substituted or unsubstituted heteroaryl.

R^{27a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{28a}-substituted or unsubstituted alkyl, R^{28a}-substituted or unsubstituted heteroalkyl, R^{28a}-substituted or unsubstituted cycloalkyl, R^{28a}-substituted or unsubstituted heterocycloalkyl, R^{28a}-substituted or unsubstituted aryl, or R^{28a}-substituted or unsubstituted heteroaryl.

In embodiments, R^{6b} is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{6b} is hydrogen, substituted alkyl, substituted heteroalkyl, substituted cycloalkyl, substituted heterocycloalkyl, substituted aryl, or substituted heteroaryl. In embodiments, R^{6b} is hydrogen, unsubstituted alkyl, unsubstituted heteroalkyl, unsubstituted cycloalkyl, unsubstituted heterocycloalkyl, unsubstituted aryl, or unsubstituted heteroaryl. In embodiments, R^{6b} is hydrogen. In embodiments, R^{6b} is unsubstituted methyl. In embodiments, R^{6b} is unsubstituted ethyl. In embodiments, R^{6b} is unsubstituted propyl.

In embodiments, R^{6b} is hydrogen, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R^{6b} is hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 9 membered heteroaryl.

In some embodiments of the compounds provided herein, R^{6b} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{26b}-substituted or unsubstituted alkyl, R^{26b}-substituted or unsubstituted heteroalkyl, R^{26b}-substituted or unsubstituted cycloalkyl, R^{26b}-substituted or unsubstituted heterocycloalkyl, R^{26b}-substituted or unsubstituted aryl, or R^{26b}-substituted or unsubstituted heteroaryl.

R^{26b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{27b}-substituted or unsubstituted alkyl, R^{27b}-substituted or unsubstituted heteroalkyl, R^{27b}-substituted or unsubstituted cycloalkyl, R^{27b}-substituted or unsubstituted heterocycloalkyl, R^{27b}-substituted or unsubstituted aryl, or R^{27b}-substituted or unsubstituted heteroaryl.

R^{27b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{28b}-substituted or unsubstituted alkyl, R^{28b}-substituted or unsubstituted heteroalkyl, R^{28b}-substituted or unsubstituted cycloalkyl, R^{28b}-substituted or unsubstituted heterocycloalkyl, R^{28b}-substituted or unsubstituted aryl, or R^{28b}-substituted or unsubstituted heteroaryl.

In embodiments, each R⁷, R⁸, R⁹, R¹⁰, R^{7a}, R^{8a}, R^{9a}, R^{10a}, R^{7b}, R^{8b}, R^{9b} and R^{10b} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, each R⁷, R⁸, R⁹, R¹⁰, R^{7a}, R^{8a}, R^{9a}, R^{10a}, R^{7b}, R^{8b}, R^{9b} and R^{10b} is independently hydrogen, substituted alkyl, substituted heteroalkyl, substituted cycloalkyl, substituted heterocycloalkyl, substituted aryl, or substituted heteroaryl. In embodiments, each R⁷, R⁸, R⁹, R¹⁰, R^{7a}, R^{8a}, R^{9a}, R^{10a}, R^{7b}, R^{8b}, R^{9b} and R^{10b} is independently hydrogen, unsubstituted alkyl, unsubstituted heteroalkyl, unsubstituted cycloalkyl, unsubstituted heterocycloalkyl, unsubstituted aryl, or unsubstituted heteroaryl. In embodiments, each R⁷, R⁸, R⁹, R¹⁰, R^{7a}, R^{8a}, R^{9a}, R^{10a}, R^{7b}R^{8b}, R^{9b} and R^{10b} is independently hydrogen.

In embodiments, each R⁷, R⁸, R⁹, R¹⁰, R^{7a}, R^{8a}, R^{9a}, R^{10a}, R^{7b}, R^{8b}, R^{9b} and R^{10b} is independently hydrogen, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, each R⁷, R⁸, R⁹, R¹⁰, R^{7a}, R^{8a}, R^{9a}, R^{10a}, R^{7b}, R^{8b}, R^{9b} and R^{10b} is independently hydrogen, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted C₆-C₁₀ aryl, or substituted or unsubstituted 5 to 9 membered heteroaryl.

In some embodiments, R⁷ is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl. In embodiments, R⁷ and R⁸ substituents bonded to the same nitrogen atom may be joined to form an R²⁹-substituted or unsubstituted heterocycloalkyl or R²⁹-substituted or unsubstituted heteroaryl.

R²⁹ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁰-substituted or unsubstituted alkyl, R³⁰-substituted or unsubstituted heteroalkyl, R³⁰-substituted or unsubstituted cycloalkyl, R³⁰-substituted or unsubstituted heterocycloalkyl, R³⁰-substituted or unsubstituted aryl, or R³⁰-substituted or unsubstituted heteroaryl.

R³⁰ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl.

In some embodiments, R^{7a} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{29a}-substituted or unsubstituted alkyl, R^{29a}-substituted or unsubstituted heteroalkyl, R^{29a}-substituted or unsubstituted cycloalkyl, R^{29a}-substituted or unsubstituted heterocycloalkyl, R^{29a}-substituted or unsubstituted aryl, or R^{29a}-substituted or unsubstituted heteroaryl. In embodiments, R^{7a} and R^{8a} substituents bonded to the same nitrogen atom may be joined to form an R^{29a}-substituted or unsubstituted heterocycloalkyl or R^{29a}-substituted or unsubstituted heteroaryl.

R^{29a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{30a}-substituted or unsubstituted alkyl, R^{30a}-substituted or unsubstituted heteroalkyl, R^{30a}-substituted or unsubstituted cycloalkyl, R^{30a}-substituted or unsubstituted heterocycloalkyl, R^{30a}-substituted or unsubstituted aryl, or R^{30a}-substituted or unsubstituted heteroaryl.

R^{30a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{31a}-substituted or unsubstituted alkyl, R^{31a}-substituted or unsubstituted heteroalkyl, R^{31a}-substituted or unsubstituted cycloalkyl, R^{31a}-substituted or unsubstituted heterocycloalkyl, R^{31a}-substituted or unsubstituted aryl, or R^{31a}-substituted or unsubstituted heteroaryl.

In some embodiments, R^{7b} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{29b}-substituted or unsubstituted alkyl, R^{29b}-substituted or unsubstituted heteroalkyl, R^{29b}-substituted or unsubstituted cycloalkyl, R^{29b}-substituted or unsubstituted heterocycloalkyl, R^{29b}-substituted or unsubstituted aryl, or R^{29b}-substituted or unsubstituted heteroaryl. In embodiments, R^{7b} and R^{8b} substituents bonded to the same nitrogen atom may be joined to form an R^{29b}-substituted or unsubstituted heterocycloalkyl or R^{29b}-substituted or unsubstituted heteroaryl.

R^{29b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{3b}-substituted or unsubstituted alkyl, R^{3b}-substituted or unsubstituted heteroalkyl, R^{3b}-substituted or unsubstituted cycloalkyl, R^{3b}-substituted or unsubstituted heterocycloalkyl, R^{3b}-substituted or unsubstituted aryl, or R^{3b}-substituted or unsubstituted heteroaryl.

R^{30b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{31b}-substituted or unsubstituted alkyl, R^{31b}-substituted or unsubstituted heteroalkyl, R^{31b}-substituted or unsubstituted cycloalkyl, R^{31b}-substituted or unsubstituted heterocycloalkyl, R^{31b}-substituted or unsubstituted aryl, or R^{31b}-substituted or unsubstituted heteroaryl.

In some embodiments, R⁸ is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³²-substituted or unsubstituted alkyl, R³²-substituted or unsubstituted heteroalkyl, R³²-substituted or unsubstituted cycloalkyl, R³²-substituted or unsubstituted heterocycloalkyl, R³²-substituted or unsubstituted aryl, or R³²-substituted or unsubstituted heteroaryl. In embodiments, R⁷ and R⁸ substituents bonded to the same nitrogen atom may be joined to form an R³²-substituted or unsubstituted heterocycloalkyl or R³²-substituted or unsubstituted heteroaryl.

R³² is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³³-substituted or unsubstituted alkyl, R³³-substituted or unsubstituted heteroalkyl, R³³-substituted or unsubstituted cycloalkyl, R³³-substituted or unsubstituted heterocycloalkyl, R³³-substituted or unsubstituted aryl, or R³³-substituted or unsubstituted heteroaryl.

R³³ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl.

In some embodiments, R^{8a} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{32a}-substituted or unsubstituted alkyl, R^{32a}-substituted or unsubstituted heteroalkyl, R^{32a}-substituted or unsubstituted cycloalkyl, R^{32a}-substituted or unsubstituted heterocycloalkyl, R^{32a}-substituted or unsubstituted aryl, or R^{32a}-substituted or unsubstituted heteroaryl. In embodiments, R^{7a} and R^{8a} substituents bonded to the same nitrogen atom may be joined to form an R^{32a}-substituted or unsubstituted heterocycloalkyl or R^{32a}-substituted or unsubstituted heteroaryl.

R^{32a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{33a}-substituted or unsubstituted alkyl, R^{33a}-substituted or unsubstituted heteroalkyl, R^{33a}-substituted or unsubstituted cycloalkyl, R^{33a}-substituted or unsubstituted heterocycloalkyl, R^{33a}-substituted or unsubstituted aryl, or R^{33a}-substituted or unsubstituted heteroaryl.

R^{33a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{34a}-substituted or unsubstituted alkyl, R^{34a}-substituted or unsubstituted heteroalkyl, R^{34a}-substituted or unsubstituted cycloalkyl, R^{34a}-substituted or unsubstituted heterocycloalkyl, R^{34a}-substituted or unsubstituted aryl, or R^{34a}-substituted or unsubstituted heteroaryl.

In some embodiments, R^{8b} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{32b}-substituted or unsubstituted alkyl, R^{32b}-substituted or unsubstituted heteroalkyl, R^{32b}-substituted or unsubstituted cycloalkyl, R^{32b}-substituted or unsubstituted heterocycloalkyl, R^{32b}-substituted or unsubstituted aryl, or R^{32b}-substituted or unsubstituted heteroaryl. In embodiments, R^{7b} and R^{8b} substituents bonded to the same nitrogen atom may be joined to form an R^{32b}-substituted or unsubstituted heterocycloalkyl or R^{32b}-substituted or unsubstituted heteroaryl.

R^{32b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{33b}-substituted or unsubstituted alkyl, R^{33b}-substituted or unsubstituted heteroalkyl, R^{33b}-substituted or unsubstituted cycloalkyl, R^{33b}-substituted or unsubstituted heterocycloalkyl, R^{33b}-substituted or unsubstituted aryl, or R^{33b}-substituted or unsubstituted heteroaryl.

R^{33b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{34b}-substituted or unsubstituted alkyl, R^{34b}-substituted or unsubstituted heteroalkyl, R^{34b}-substituted or unsubstituted cycloalkyl, R^{34b}-substituted or unsubstituted heterocycloalkyl, R^{34b}-substituted or unsubstituted aryl, or R^{34b}-substituted or unsubstituted heteroaryl.

In some embodiments, R⁹ is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁵-substituted or unsubstituted alkyl, R³⁵-substituted or unsubstituted heteroalkyl, R³⁵-substituted or unsubstituted cycloalkyl, R³⁵-substituted or unsubstituted heterocycloalkyl, R³⁵-substituted or unsubstituted aryl, or R³⁵-substituted or unsubstituted heteroaryl.

R³⁵ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁶-substituted or unsubstituted alkyl, R³⁶-substituted or unsubstituted heteroalkyl, R³⁶-substituted or unsubstituted cycloalkyl, R³⁶-substituted or unsubstituted heterocycloalkyl, R³⁶-substituted or unsubstituted aryl, or R³⁶-substituted or unsubstituted heteroaryl.

R³⁶ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁷-substituted or unsubstituted alkyl, R³⁷-substituted or unsubstituted heteroalkyl, R³⁷-substituted or unsubstituted cycloalkyl, R³⁷-substituted or unsubstituted heterocycloalkyl, R³⁷-substituted or unsubstituted aryl, or R³⁷-substituted or unsubstituted heteroaryl.

In some embodiments, R^{9a} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{35a}-substituted or unsubstituted alkyl, R^{35a}-substituted or unsubstituted heteroalkyl, R^{35a}-substituted or unsubstituted cycloalkyl, R^{35a}-substituted or unsubstituted heterocycloalkyl, R^{35a}-substituted or unsubstituted aryl, or R^{35a}-substituted or unsubstituted heteroaryl.

R^{35a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{36a}-substituted or unsubstituted alkyl, R^{36a}-substituted or unsubstituted heteroalkyl, R^{36a}-substituted or unsubstituted cycloalkyl, R^{36a}-substituted or unsubstituted heterocycloalkyl, R^{36a}-substituted or unsubstituted aryl, or R^{36a}-substituted or unsubstituted heteroaryl.

R^{36a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{37a}-substituted or unsubstituted alkyl, R^{37a}-substituted or unsubstituted heteroalkyl, R^{37a}-substituted or unsubstituted cycloalkyl, R^{37a}-substituted or unsubstituted heterocycloalkyl, R^{37a}-substituted or unsubstituted aryl, or R^{37a}-substituted or unsubstituted heteroaryl.

In some embodiments, R^{9b} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{35b}-substituted or unsubstituted alkyl, R^{35b}-substituted or unsubstituted heteroalkyl, R^{35b}-substituted or unsubstituted cycloalkyl, R^{35b}-substituted or unsubstituted heterocycloalkyl, R^{35b}-substituted or unsubstituted aryl, or R^{35b}-substituted or unsubstituted heteroaryl.

R^{35b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{36b}-substituted or unsubstituted alkyl, R^{36b}-substituted or unsubstituted heteroalkyl, R^{36b}-substituted or unsubstituted cycloalkyl, R^{36b}-substituted or unsubstituted heterocycloalkyl, R^{36b}-substituted or unsubstituted aryl, or R^{36b}-substituted or unsubstituted heteroaryl.

R^{36b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{37b}-substituted or unsubstituted alkyl, R^{37b}-substituted or unsubstituted heteroalkyl, R^{37b}-substituted or unsubstituted cycloalkyl, R^{37b}-substituted or unsubstituted heterocycloalkyl, R^{37b}-substituted or unsubstituted aryl, or R^{37b}-substituted or unsubstituted heteroaryl.

In some embodiments, R¹⁰ is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁸-substituted or unsubstituted alkyl, R³⁸-substituted or unsubstituted heteroalkyl, R³⁸-substituted or unsubstituted cycloalkyl, R³⁸-substituted or unsubstituted heterocycloalkyl, R³⁸-substituted or unsubstituted aryl, or R³⁸-substituted or unsubstituted heteroaryl.

R³⁸ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R³⁹-substituted or unsubstituted alkyl, R³⁹-substituted or unsubstituted heteroalkyl, R³⁹-substituted or unsubstituted cycloalkyl, R³⁹-substituted or unsubstituted heterocycloalkyl, R³⁹-substituted or unsubstituted aryl, or R³⁹-substituted or unsubstituted heteroaryl.

R³⁹ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴⁰-substituted or unsubstituted alkyl, R⁴⁰-substituted or unsubstituted heteroalkyl, R⁴⁰-substituted or unsubstituted cycloalkyl, R⁴⁰-substituted or unsubstituted heterocycloalkyl, R⁴⁰-substituted or unsubstituted aryl, or R⁴⁰-substituted or unsubstituted heteroaryl.

In some embodiments, R^{10a} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{38a}-substituted or unsubstituted alkyl, R^{38a}-substituted or unsubstituted heteroalkyl, R^{38a}-substituted or unsubstituted cycloalkyl, R^{38a}-substituted or unsubstituted heterocycloalkyl, R^{38a}-substituted or unsubstituted aryl, or R^{38a}-substituted or unsubstituted heteroaryl.

R^{38a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{39a}-substituted or unsubstituted alkyl, R^{39a}-substituted or unsubstituted heteroalkyl, R^{39a}-substituted or unsubstituted cycloalkyl, R^{39a}-substituted or unsubstituted heterocycloalkyl, R^{39a}-substituted or unsubstituted aryl, or R^{39a}-substituted or unsubstituted heteroaryl.

R^{39a} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{40a}-substituted or unsubstituted alkyl, R^{40a}-substituted or unsubstituted heteroalkyl, R^{40a}-substituted or unsubstituted cycloalkyl, R^{40a}-substituted or unsubstituted heterocycloalkyl, R^{40a}-substituted or unsubstituted aryl, or R^{40a}-substituted or unsubstituted heteroaryl.

In some embodiments, R^{10b} is independently hydrogen, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{38b}-substituted or unsubstituted alkyl, R^{38b}-substituted or unsubstituted heteroalkyl, R^{38b}-substituted or unsubstituted cycloalkyl, R^{38b}-substituted or unsubstituted heterocycloalkyl, R^{38b}-substituted or unsubstituted aryl, or R^{38b}-substituted or unsubstituted heteroaryl.

R^{38b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{39b}-substituted or unsubstituted alkyl, R^{39b}-substituted or unsubstituted heteroalkyl, R^{39b}-substituted or unsubstituted cycloalkyl, R^{39b}-substituted or unsubstituted heterocycloalkyl, R^{39b}-substituted or unsubstituted aryl, or R^{39b}-substituted or unsubstituted heteroaryl.

R^{39b} is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{4b}-substituted or unsubstituted alkyl, R^{4b}-substituted or unsubstituted heteroalkyl, R^{4b}-substituted or unsubstituted cycloalkyl, R^{4b}-substituted or unsubstituted heterocycloalkyl, R^{4b}-substituted or unsubstituted aryl, or R^{4b}-substituted or unsubstituted heteroaryl.

In embodiments, v is 1. In embodiments, v is 2. In embodiments, v1 is 1. In embodiments, v1 is 2. In embodiments, v2 is 1. In embodiments, v2 is 2. In embodiments, m is 1. In embodiments, m is 2. In embodiments, ml is 1. In embodiments, ml is 2. In embodiments, m2 is 1. In embodiments, m2 is 2. In embodiments, n is independently 0. In embodiments, n is independently 1. In embodiments, n is independently 2. In embodiments, n is independently 3. In embodiments, n is independently 4. In embodiments, n1 is independently 0. In embodiments, n1 is independently 1. In embodiments, n1 is independently 2. In embodiments, n1 is independently 3. In embodiments, n1 is independently 4. In embodiments, n2 is independently 0. In embodiments, n2 is independently 1. In embodiments, n2 is independently 2. In embodiments, n2 is independently 3. In embodiments, n2 is independently 4. In embodiments, X is —Cl. In embodiments, X is —Br. In embodiments, X is —I. In embodiments, X is —F. In embodiments, X^{a} is —Cl. In embodiments, X^{a} is —Br. In embodiments, X^{a} is —I. In embodiments, X^{a} is —F. In embodiments, X^{b} is —Cl. In embodiments, X^{b} is —Br. In embodiments, X^{b} is —I. In embodiments, X^{b} is —F. In embodiments, z is 0. In embodiments, z is 1. In embodiments, z is 2. In embodiments, z2 is 1. In embodiments, z2 is 2. In embodiments, z2 is 3. In embodiments, z2 is 4.

In embodiments, the compound has the formula:

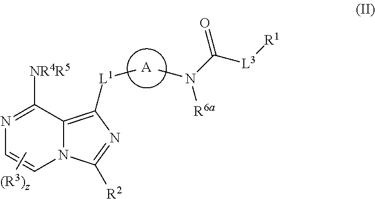

wherein, R¹, R², R³, R⁴, R⁵, R^{6a}, L¹, ring A and z are as described herein (e.g. compounds of formula I, including embodiments).

L³ is a bond, —O—, —NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is a bond. In embodiments, L³ is —NR^{6b}—, wherein R^{6b} is as defined herein including embodiments thereof. In embodiments, L³ is —NH—. In embodiments, L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is —O—. In embodiments, L³ is —OCH₂—.

In embodiments, L³ is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³ is substituted alkylene, substituted heteroalkylene, substituted cycloalkylene, substituted heterocycloalkylene, substituted arylene, or substituted heteroarylene. In embodiments, L³ is unsubstituted alkylene, unsubstituted heteroalkylene, unsubstituted cycloalkylene, unsubstituted heterocycloalkylene, unsubstituted arylene, or unsubstituted heteroarylene.

In embodiments, L³ is substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, L³ is substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted C₆-C₁₀ arylene, or substituted or unsubstituted 5 to 9 membered heteroarylene.

In some embodiments, L³ is independently R⁴⁷-substituted or unsubstituted alkylene, R⁴⁷-substituted or unsubstituted heteroalkylene, R⁴⁷-substituted or unsubstituted cycloalkylene, R⁴⁷-substituted or unsubstituted heterocycloalkylene, R⁴⁷-substituted or unsubstituted arylene, or R⁴⁷-substituted or unsubstituted heteroarylene.

R⁴⁷ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴⁸-substituted or unsubstituted alkyl, R⁴⁸-substituted or unsubstituted heteroalkyl, R⁴⁸-substituted or unsubstituted cycloalkyl, R⁴⁸-substituted or unsubstituted heterocycloalkyl, R⁴⁸-substituted or unsubstituted aryl, or R⁴⁸-substituted or unsubstituted heteroaryl.

R⁴⁸ is independently oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl.

Each R¹³, R¹⁶, R¹⁹, R^{28a}, R^{28b}, R³¹, R^{31a}, R^{31b}, R³⁴, R^{34a}, R^{34b}, R³⁷, R^{37a}, R^{37b}, R⁴⁰, R^{40a}, R^{40b}, R⁴³, R⁴⁶, and R⁴⁹ is independently a hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, each R¹³, R¹⁶, R¹⁹, R^{28a}, R^{28b}, R³¹, R^{31a}, R^{31b}, R³⁴, R^{34a}, R^{34b}, R³⁷, R^{37a}, R^{37b}, R⁴⁰, R^{40a}, R^{40b}, R⁴³, R⁴⁶, and R⁴⁹ is independently hydrogen, unsubstituted C₁-C₈ alkyl, unsubstituted 2 to 8 membered heteroalkyl, unsubstituted C₃-C₈ cycloalkyl, unsubstituted 3 to 8 membered heterocycloalkyl, unsubstituted C₆-C₁₀ aryl, or unsubstituted 5 to 10 membered heteroaryl. In embodiments, each R¹³, R¹⁶, R¹⁹, R^{28a}, R^{28b}, R³¹, R^{31a}, R^{31b}, R³⁴, R^{34a}, R^{34b}, R³⁷, R^{37a}, R^{37b}, R⁴⁰, R^{40a}, R^{40b}, R⁴³, R⁴⁶, and R⁴⁹ is independently hydrogen, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted C₆-C₁₀ aryl, or unsubstituted 5 to 9 membered heteroaryl. In embodiments, each R¹³, R¹⁶, R¹⁹, R^{28a}, R^{28b}, R³¹, R^{31a}, R^{31b}, R³⁴, R^{34a}, R^{34b}, R³⁷, R^{37a}, R^{37b}, R⁴⁰, R^{40a}, R^{40b}, R⁴³, R⁴⁶, and R⁴⁹ is hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, or —OCHF₂.

In embodiments, the compound has the formula:

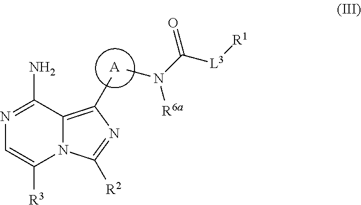

wherein, R¹, R², R³, R^{6a}, L³, and ring A are as described herein (e.g. compounds of formula I or II, including embodiments).

In embodiments, the compound has the formula:

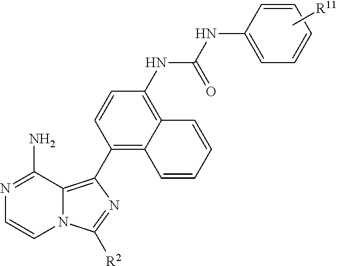

wherein, R¹¹ and R² are as described herein (e.g. compounds of formula I, II, III, including embodiments). In embodiments, R¹¹ is —CF₃ or halogen. In embodiments, R¹¹ is meta —CF₃. In embodiments, R¹¹ is meta —F. In embodiments, R¹¹ meta —Cl. In embodiments, R¹¹ is meta —Br. In embodiments, R¹¹ is meta —I. In embodiments, R¹¹ is meta —CH₃. In embodiments, R¹¹ is meta —CCl₃. In embodiments, R¹¹ is meta —CF₃, —Cl, —OCF₃, —CH₃, —F, —OCH₃, —OPh, —CH₂Ph, or —CHO. In embodiments, R¹¹ is meta —CF₃, —Cl, —OCF₃, —CH₃, —F, —OCH₃, —OPh, —CH₂Ph, or —CHO. In embodiments, R¹¹ is ortho —CF₃, —Cl, —OCF₃, —CH₃, —F, —OCH₃, —OPh, —CH₂Ph, or —CHO. In embodiments, R¹¹ is meta —CF₃, —Cl, —OCF₃, —CH₃, —F, —OCH₃, —OPh, —CH₂Ph, or —CHO. In embodiments, R¹¹ is ortho —CF₃, —Cl, —OCF₃, —CH₃, —F, —OCH₃, —OPh, —CH₂Ph, or —CHO. In embodiments, R¹¹ is para-CF₃, —Cl, —OCF₃, —CH₃, —F, —OCH₃, —OPh, —CH₂Ph, or —CHO. In embodiments, R¹¹ is —CF₃, —Cl, —OCF₃, —CH₃, —F, —OCH₃, —OPh, —CH₂Ph, or —CHO. In embodiments, R² is unsubstituted C₁-C₄ alkyl.

In embodiments, the compound is:

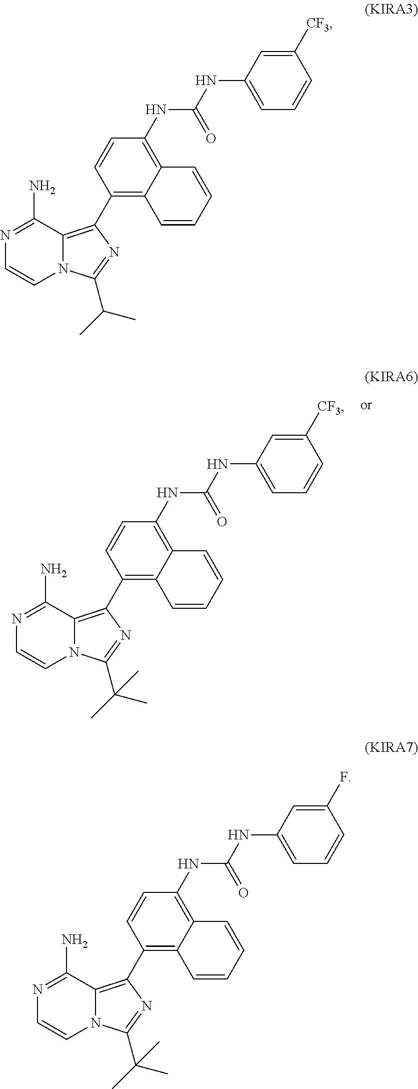 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "or" ; CC(C)(C)c1nc(-c2ccc(NC(=O)Nc3cccc(F)c3)c3ccccc23)c2c(N)nccn12]

In embodiments, the compound is an inhibitor of Ire1. In embodiments, the compound is an inhibitor of Ire1α. In embodiments, the compound is an inhibitor of Ire1α kinase activity. In embodiments, the compound is an inhibitor of Ire1α RNase activity. In embodiments, the compound binds the ATP binding site of Ire1α. In embodiments, the compound binds Ire1α in the DFG-out conformation. In embodiments, the compound induces the DFG-out conformation of Ire1α. In embodiments, the compound is an inhibitor of Ire1α oligomerization. In embodiments, the compound is an inhibitor of Ire1α dimerization. In embodiments, the compound is an inhibitor of Ire1α phosphorylation. In embodiments, the compound is an inhibitor of Ire1α autophosphorylation. In embodiments, the compound is an inhibitor of apoptosis. In embodiments, the compound is an inhibitor of Ire1α induced apoptosis. In embodiments, the compound is an inhibitor of cell death. In embodiments, the compound is an inhibitor of Ire1α induced cell death. In embodiments, the compound is an inhibitor of a pathway induced by Ire1α phosphorylation. In embodiments, the compound is an inhibitor of a pathway induced by Ire1α kinase activity. In embodiments, the compound is an inhibitor of a pathway induced by Ire1α RNase activity. In embodiments, the compound is an inhibitor of neuronal cell death. In embodiments, the compound is a cytotoxic agent. In embodiments, the compound is an anti-cancer agent. In embodiments, the compound is an inhibitor of demyelination. In embodiments, the compound is an inhibitor of diabetes. In embodiments, the compound is an anti-diabetic agent. In embodiments, the compound is a neuroprotective agent. In embodiments, the compound is an inhibitor of fibrosis. In embodiments, the compound decreases apoptosis in cells under ER stress. In embodiments, the compound decreases apoptosis in cells under ER stress but not cells under the same conditions except that they are not under ER stress. In embodiments, the compound decreases apoptosis in cells under ER stress more than in cells under the same conditions except that they are not under ER stress. In embodiments, the compound decreases cleavage of miR-17. In embodiments, the compound decreases Ire1 associated cleavage of miR-17. In embodiments, the compound decreases cleavage of miR-34a. In embodiments, the compound decreases Ire1α associated cleavage of miR-34a. In embodiments, the compound decreases cleavage of miR-96. In embodiments, the compound decreases Ire1α associated cleavage of miR-96. In embodiments, the compound decreases cleavage of miR-125b. In embodiments, the compound decreases Ire1α associated cleavage of miR-125b. In embodiments, the compound decreases XBP1 mRNA splicing. In embodiments, the compound decreases Ire1α associated XBP1 mRNA splicing. In embodiments, the compound decreases the UPR. In embodiments, the compound decreases Ire1α associated UPR. In embodiments, the compound decreases the terminal UPR. In embodiments, the compound decreases Ire1α associated terminal UPR.

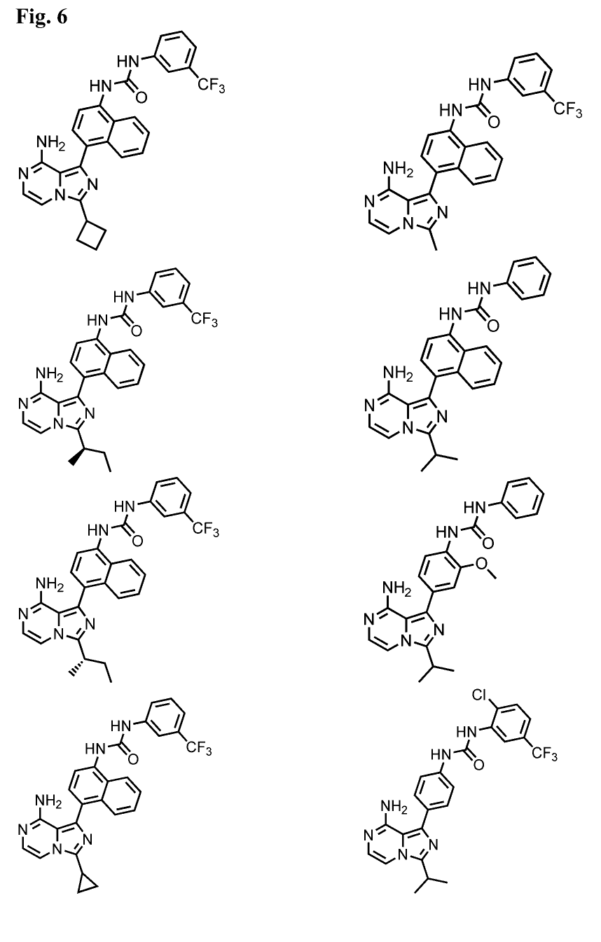

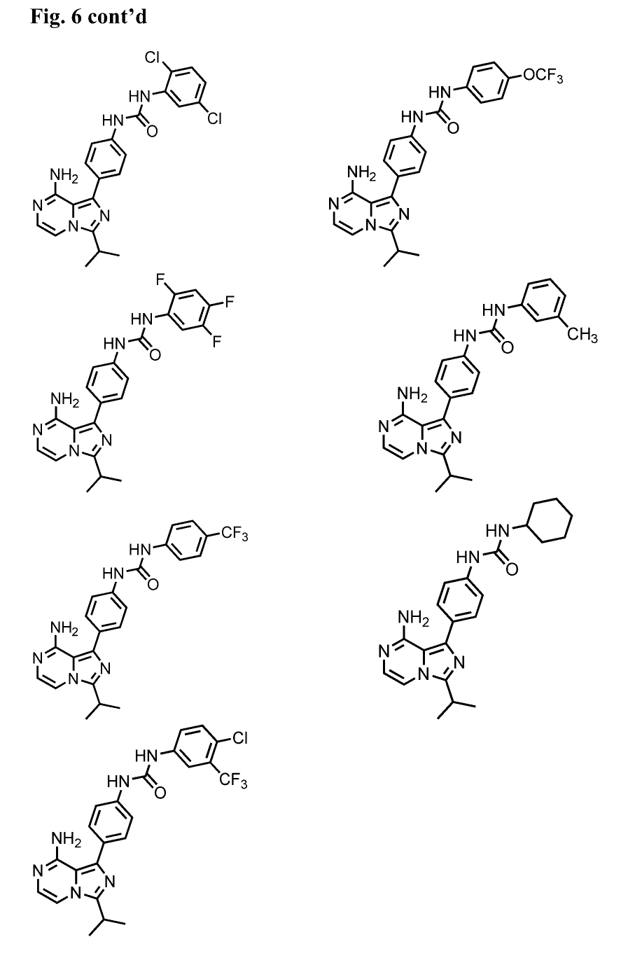

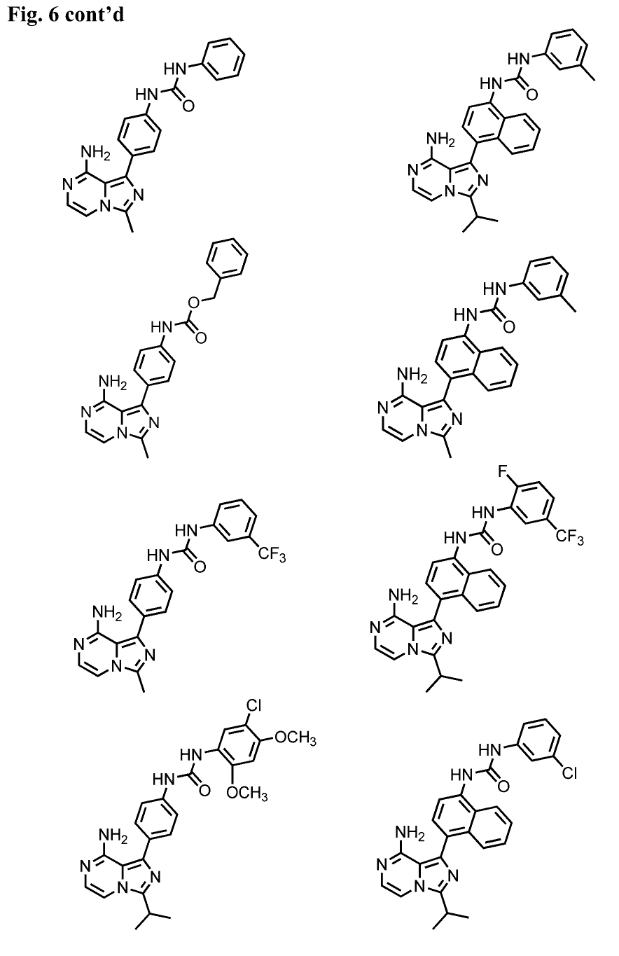

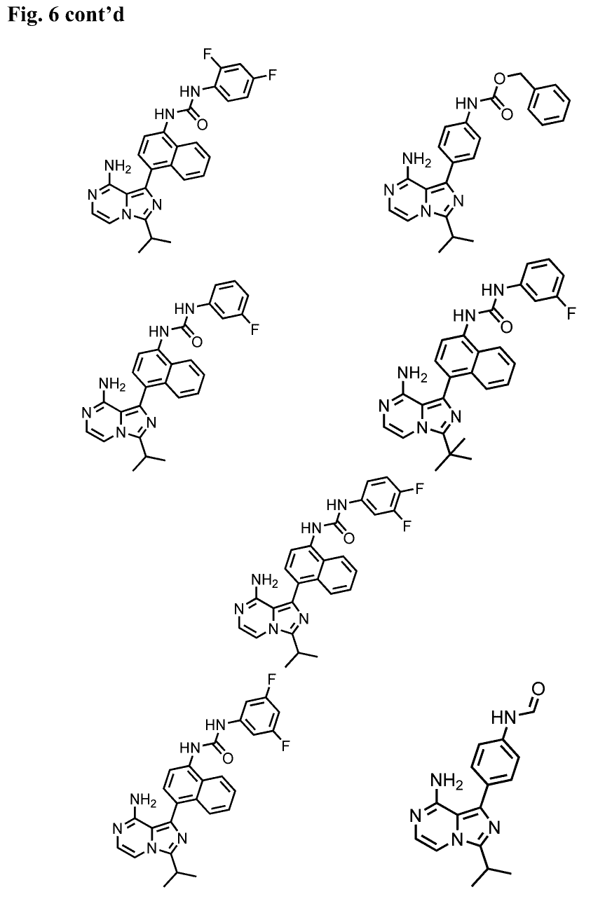

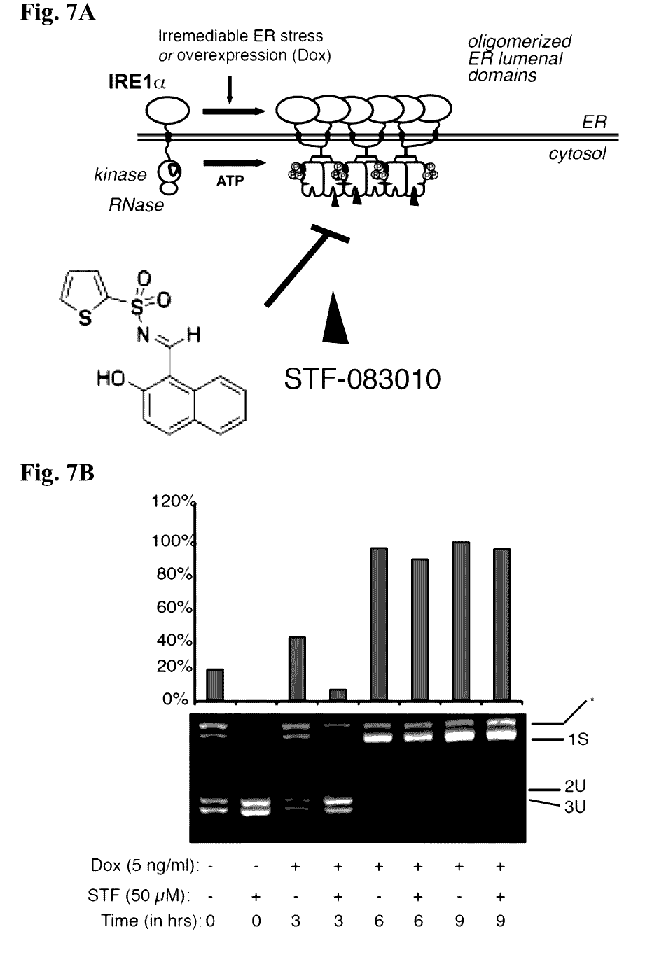

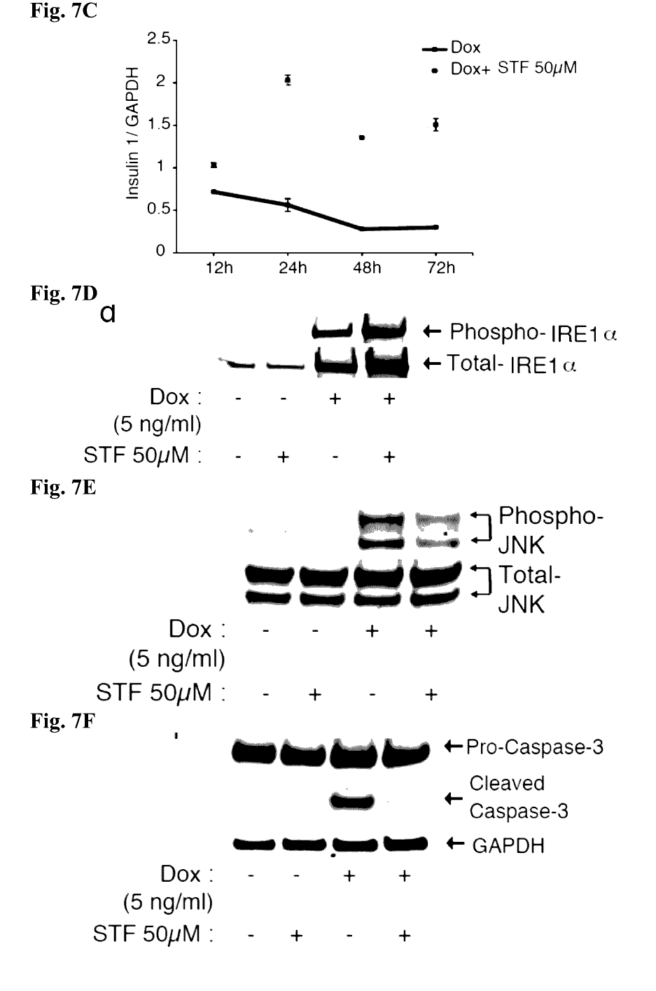

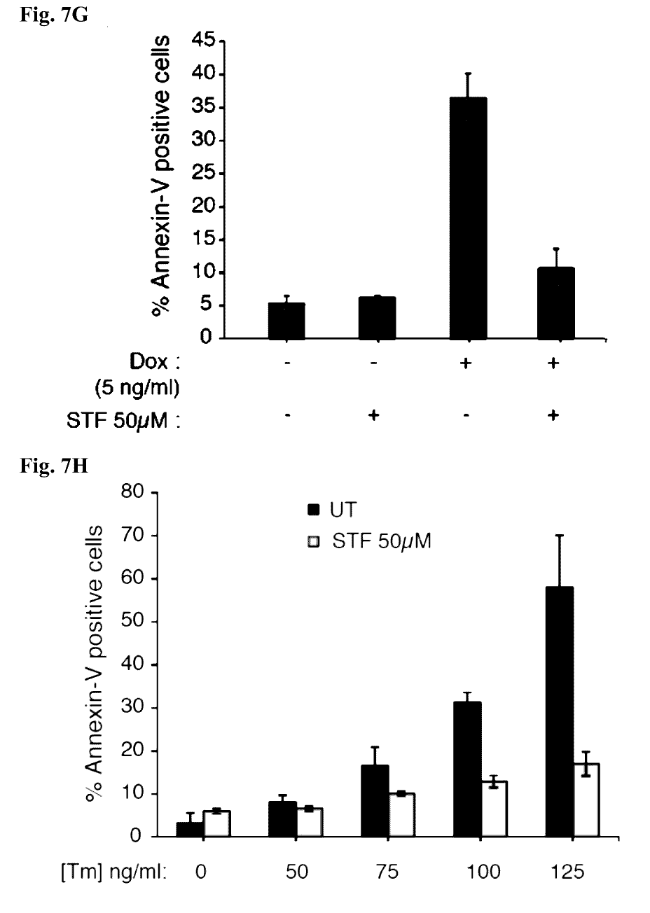

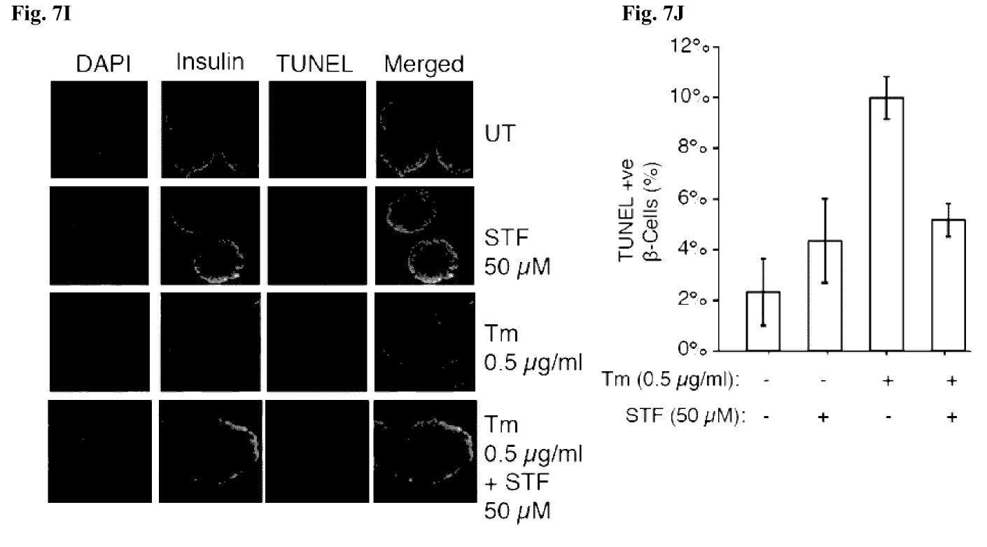

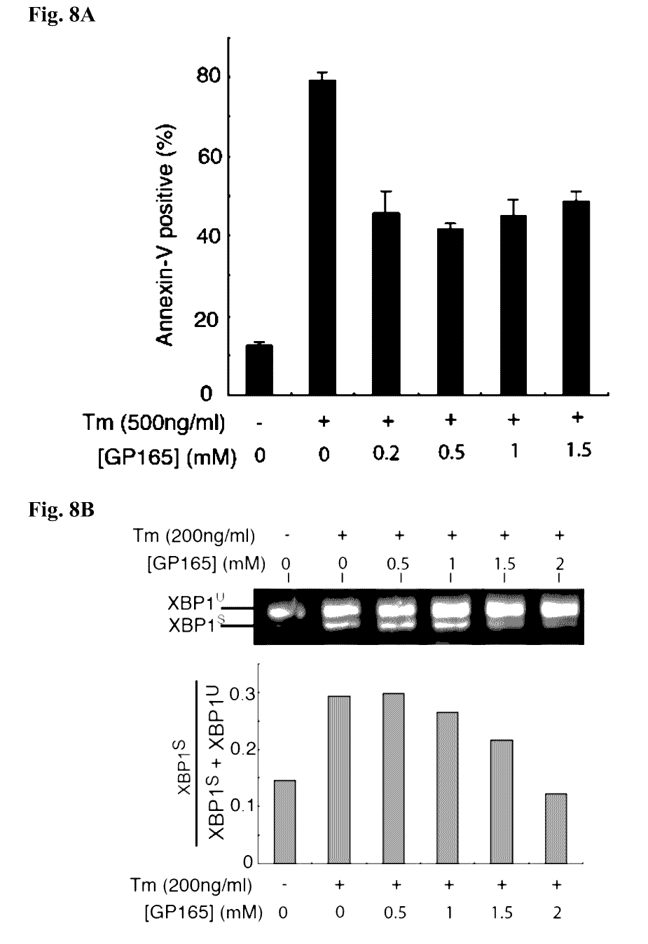

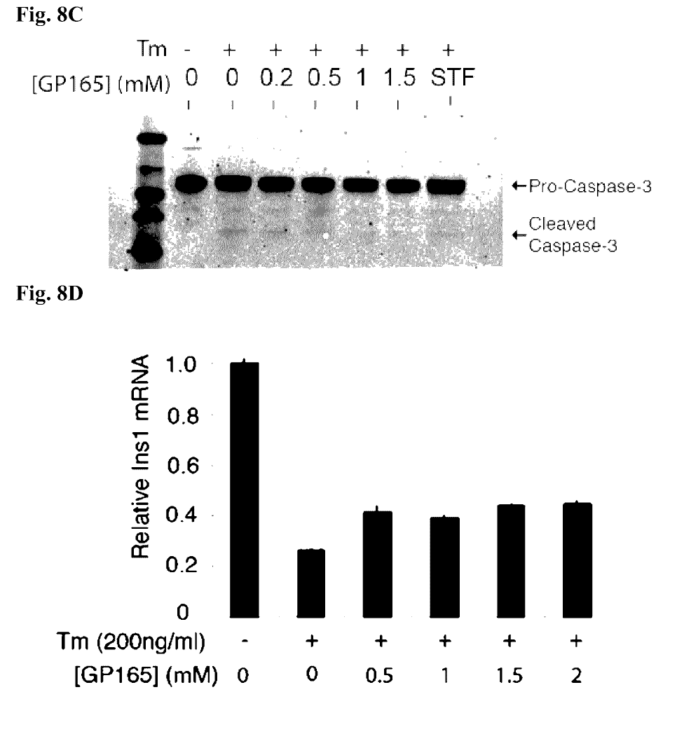

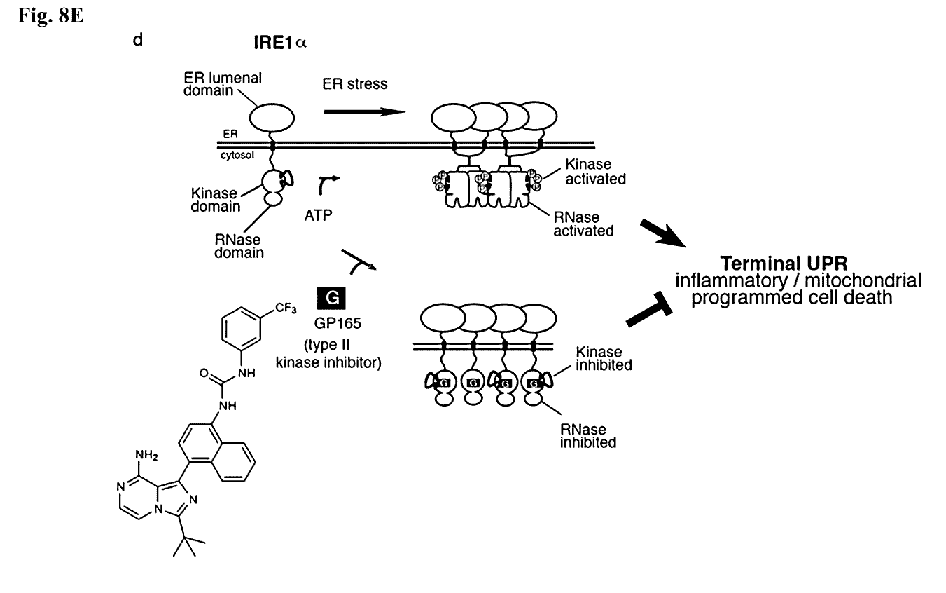

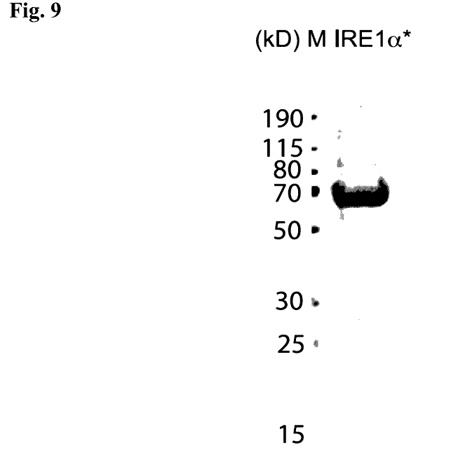

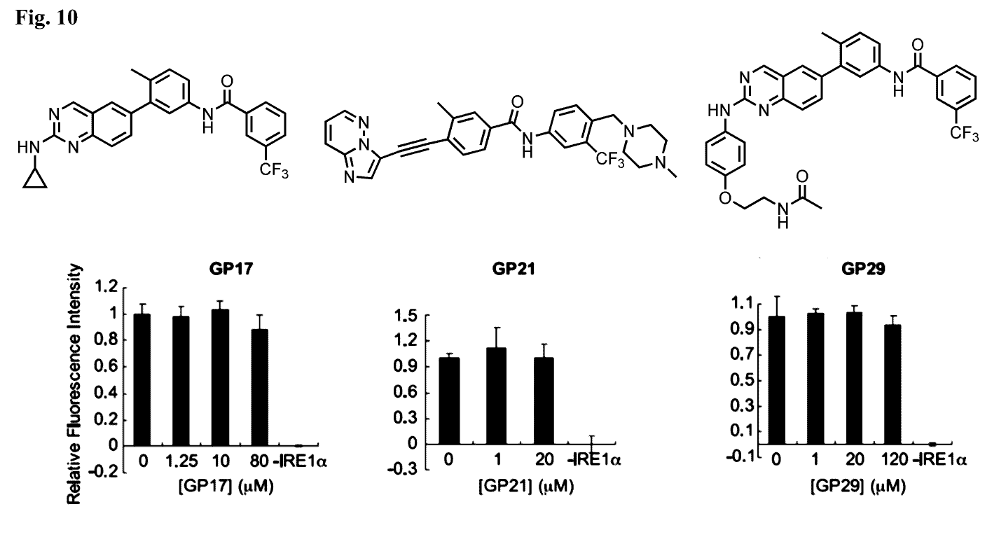

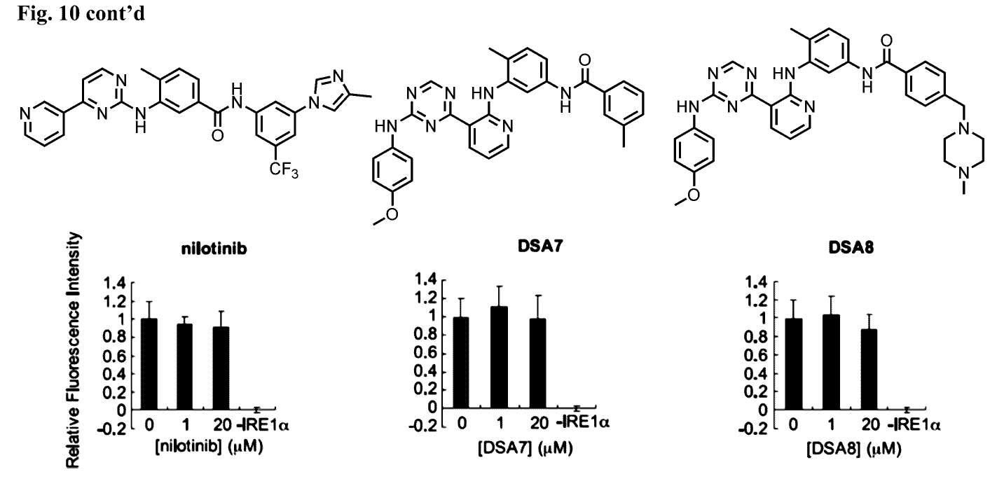

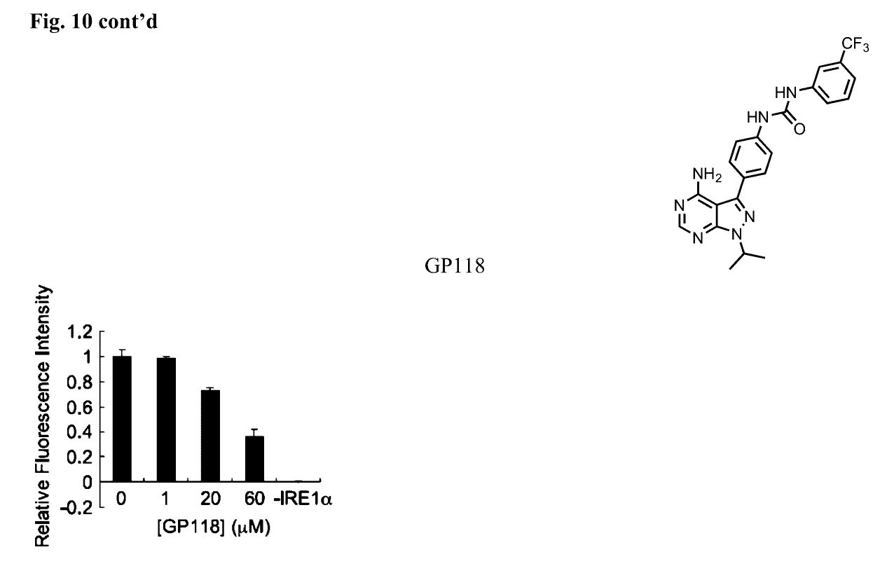

In embodiments, the compound is a compound described herein, including in an aspect, embodiment, example, figure, table, or claim. In embodiments, the compound is a compound in FIG. 6.

In embodiments, the compounds set forth herein are provided as pharmaceutical compositions including the compound, or a pharmaceutically acceptable salt thereof, and a pharmaceutically acceptable excipient. In embodiments, the compounds set forth herein are not provided as pharmaceutical compositions. In embodiments, the compound is included in a pharmaceutically acceptable salt. In embodiments, the compound is not included in a pharmaceutically acceptable salt.

Described herein, inter alia, is a new strategy to: (1) inhibit IRE1α's hyperactive RNase by pharmacologically targeting its neighboring kinase domain with small molecules, and (2) test physiological benefits of shutting down IRE1α in cells (e.g. β-cells) of living mammals (e.g. mice). This work validates IRE1α as a drug target to manipulate ER stress signaling to control cell fate.

In another aspect, provided herein are compounds having the formula (A):

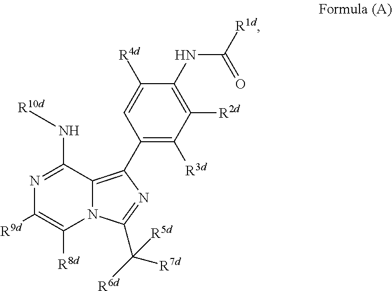

and pharmaceutically acceptable salts thereof, wherein Rid, R^{2d}, R^{3d}, R^{4d}, R^{5d}, R^{6d}, R^{7d}, R^{8d}, R^{9d}, and R^{10d}, are each independently C₂₋₆ alkyl, C₁₋₆ haloalkyl, —C₁₋₄ alkyl-R^{12d}, C₂₋₆ alkenyl, C₂₋₆ alkynyl, C₃₋₈ cycloalkyl, monocyclic heterocyclyl, monocyclic heteroaryl, or phenyl, aryl, wherein the cycloalkyl, heterocyclyl, heteroaryl, and phenyl groups are each optionally substituted with one or two R^{11d} groups; each R^{11d} is independently C₁₋₆ alkyl, C₁₋₆ haloalkyl, —C(O)R^{d}, —C(O)OR^{d}, —C(O)NR^{d}₂, S(O)₂NR^{d}₂, or —S(O)₂R^{d}; and R^{12d} is —OR^{d}, —SR^{d}, —NR^{d}₂, —C(O)R^{d}, —C(O)OR^{d}, —C(O)NR^{d}₂, —S(O)₂R^{d}, —OC(O)R^{d}, OC(O)OR^{d}, OC(O)NR^{d}₂, —N(R^{d})C(O)R^{d}, —N(R^{d})C(O)OR^{d}, —N(R^{d})C(O)NR^{d}₂, phenyl, monocyclic heteroaryl, C₃₋₈ cycloalkyl, or monocyclic heterocyclyl, wherein the aryl, heteroaryl, C₃₋₈ cycloalkyl, and heterocyclyl groups are each optionally substituted by one, two, or three groups that are each independently halogen, cyano, nitro, C₁₋₆ alkyl, C₁₋₆ haloalkyl, —OR^{d}, —SR^{d}, —NR^{d}₂, —C(O) R^{d}, C(O)OR^{d}, —C(O)NR^{d}₂, —S(O)₂R^{d}, —OC(O)R^{d}, —OC(O)OR^{d}, OC(O)NR^{d}₂, N(R^{d})C(O)R^{d}, —N(R^{d})C(O)OR^{d}, or —N(R^{d})C(O)NR^{d}₂; and each R^{d} is independently hydrogen, C₁₋₆ alkyl, C₂₋₆ alkenyl, C₁₋₆ haloalkyl, C₃₋₈ cycloalkyl, heterocyclyl, aryl, arylC₁₋₆ alkyl, heteroaryl, or heteroarylC₁₋₆ alkyl wherein the alkyl, aryl, arylalkyl, heteroaryl, and heteroarylalkyl are optionally substituted with one, two, three, or four groups that are each independently halogen, cyano, nitro, C₁₋₆ alkyl, C₁₋₆ haloalkyl, —OR^{0d}, SR^{0d}, NR^{0d}2, C(O)R^{0d}, C(O)OR^{0d}, —C(O)N(R^{0d})2, S(O)2R^{0d}, —OC(O)R^{0d}, —OC(O)OR^{0d}, OC(O)N(R^{0d})2, N(R^{0d})C(O)R^{0d}, —N(R^{0d})C(O)OR^{0d}, or N(R^{0d})C(O)N(R^{0d})2, wherein each R^{0d} is independently hydrogen or C₁₋₆ alkyl, each R^{d} is independently hydrogen, or C₁₋₆ alkyl.

In another aspect, provided herein are compounds having the formula (A):

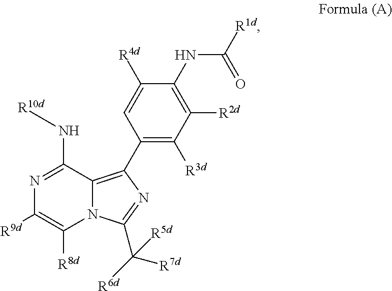

and pharmaceutically acceptable salts thereof, wherein Rid, R^{2d}, R^{3d}, R^{4d}, R^{5d}, R^{6d}, R^{7d}, R^{8d}, R^{9d} and R^{10d}, are each independently C₂₋₆ alkyl, C₁₋₆ haloalkyl, —C₁₋₄ alkyl-R^{12d}, C₂— alkenyl, C₂₋₆ alkynyl, C₃₋₈ cycloalkyl, monocyclic heterocyclyl, monocyclic heteroaryl, or phenyl, aryl, wherein the cycloalkyl, heterocyclyl, heteroaryl, and phenyl groups are each optionally substituted with one or two R^{11d} groups; each R^{11d} is independently C₁₋₆ alkyl, C₁₋₆ haloalkyl, —C(O)R^{d}, —C(O)OR^{d}, —C(O)NR^{d}₂, S(O)₂NR^{d}₂, or —S(O)₂R^{d}; and R^{12d} is —OR^{d}, —SR^{d}, —NR^{d}₂, —C(O)R^{d}, —C(O)OR^{d}, —C(O)NR^{d}₂, —S(O)₂R^{d}, —OC(O)R^{d}, OC(O)OR^{d}, OC(O)NR^{d}₂, —N(R^{d})C(O)R^{d}, —N(R^{d})C(O)OR^{d}, —N(R^{d})C(O)NR^{d}₂, phenyl, monocyclic heteroaryl, C₃₋₈ cycloalkyl, or monocyclic heterocyclyl, wherein the aryl, heteroaryl, C₃₋₈ cycloalkyl, and heterocyclyl groups are each optionally substituted by one, two, or three groups that are each independently halogen, cyano, nitro, C₁₋₆ alkyl, C₁₋₆ haloalkyl, —OR^{d}, —SR^{d}, —NR^{d}₂, —C(O) R^{d}, C(O)OR^{d}, —C(O)NR^{d}₂, —S(O)₂R^{d}, —OC(O)R^{d}, —OC(O)OR^{d}, OC(O)NR^{d}₂, N(R^{d})C(O)R^{d}, —N(R^{d})C(O)OR^{d}, or —N(R^{d})C(O)NR^{d}₂; and each R^{d} is independently hydrogen, C₁₋₆ alkyl, C₂₋₆ alkenyl, C₁₋₆ haloalkyl, C₃₋₈ cycloalkyl, heterocyclyl, aryl, arylC₁₋₆ alkyl, heteroaryl, or heteroarylC₁₋₆ alkyl wherein the alkyl, aryl, arylalkyl, heteroaryl, and heteroarylalkyl are optionally substituted with one, two, three, or four groups that are each independently halogen, cyano, nitro, C₁₋₆ alkyl, C₁₋₆ haloalkyl, —OR^{0d}, SR^{0d}, NR^{0d}2, C(O)R^{0d}, C(O)OR^{0d}, —C(O)N(R^{0d})2, S(O)2R^{0d}, —OC(O)R^{0d}, —OC(O)OR^{0d}, OC(O)N(R^{0d})2, N(R^{0d})C(O)R^{0d}, —N(R^{0d})C(O)OR^{0d}, or N(R^{0d})C(O)N(R^{0d})2, wherein each R^{0d} is independently hydrogen or C₁₋₆ alkyl. In embodiments, each R^{d} is independently hydrogen or C₁₋₆ alkyl.

In another aspect, R^{2d} and R^{3d} are together a phenyl, monocyclic heteroaryl, C₃₋₈ cycloalkyl, or monocyclic heterocyclyl, wherein the aryl, heteroaryl, C₃₋₈ cycloalkyl, and heterocyclyl groups are each optionally substituted by one, two, or three groups that are each independently halogen, cyano, nitro, C₁₋₆ alkyl, C₁₋₆ haloalkyl, —OR^{d}, —SR^{d}, —NR^{d}₂, —C(O) R^{d}, C(O)OR^{d}, —C(O)NR^{d}₂, —S(O)₂R^{d}, —OC(O)R^{d}, —OC(O)OR^{d}, OC(O)NR^{d}₂, N(R^{d})C(O) R^{d}, —N(R^{d})C(O)OR^{d}, or —N(R^{d})C(O)NR^{d}₂; wherein each R^{d} is independently hydrogen, C₁₋₆ alkyl, C₂₋₆ alkenyl, C₁₋₆ haloalkyl, C₃₋₈ cycloalkyl, heterocyclyl, aryl, arylC₁₋₆ alkyl, heteroaryl, or heteroarylC₁₋₆ alkyl wherein the alkyl, aryl, arylalkyl, heteroaryl, and heteroarylalkyl are optionally substituted with one, two, three, or four groups that are each independently halogen, cyano, nitro, C₁₋₆ alkyl, C₁₋₆ haloalkyl, —OR^{0d}, SR^{0d}, NR^{0d}2, C(O)R^{0d}, C(O)OR^{0d}, —C(O)N(R^{0d})2, S(O)₂R^{0d}, —OC(O)R^{0d}, —OC(O)OR^{0d}, OC(O)N(R^{0d})2, N(R^{0d})C(O)R^{0d}, —N(R^{0d})C(O)OR^{0d}, or N(R^{0d})C(O)N(R^{0d})2, wherein each R^{0d} is independently hydrogen or C₁₋₆ alkyl, each R^{d} is independently hydrogen, or C₁₋₆ alkyl.

In another aspect, R^{2d} and R^{3d} are together a phenyl, monocyclic heteroaryl, C₃₋₈ cycloalkyl, or monocyclic heterocyclyl, wherein the aryl, heteroaryl, C₃₋₈ cycloalkyl, and heterocyclyl groups are each optionally substituted by one, two, or three groups that are each independently halogen, cyano, nitro, C₁₋₆ alkyl, C₁₋₆ haloalkyl, —OR^{d}, —SR^{d}, —NR^{d}₂, —C(O) R^{d}, C(O)OR^{d}, —C(O)NR^{d}₂, —S(O)₂R^{d}, —OC(O) R^{d}, —OC(O)OR^{d}, OC(O)NR^{d}₂, N(R^{d})C(O) R^{d}, —N(R^{d})C(O)OR^{d}, or —N(R^{d})C(O)NR^{d}₂; wherein each R^{d} is independently hydrogen, C₁₋₆ alkyl, C₂₋₆ alkenyl, C₁₋₆ haloalkyl, C₃₋₈ cycloalkyl, heterocyclyl, aryl, arylC₁₋₆ alkyl, heteroaryl, or heteroarylC₁₋₆ alkyl wherein the alkyl, aryl, arylalkyl, heteroaryl, and heteroarylalkyl are optionally substituted with one, two, three, or four groups that are each independently halogen, cyano, nitro, C₁₋₆ alkyl, C₁₋₆ haloalkyl, —OR^{0d}, SR^{0d}, NR^{0d}2, C(O)R^{0d}, C(O)OR^{0d}, —C(O)N(R^{0d})2, S(O)2R^{0d}, —OC(O)R^{0d}, —OC(O)OR^{0d}, OC(O)N(R^{0d})2, N(R^{0d})C(O)R^{0d}, —N(R^{0d})C(O)OR^{0d}, or N(R^{0d})C(O)N(R^{0d})2, wherein each R^{0d} is independently hydrogen or C₁₋₆ alkyl. In embodiments, each R^{d} is independently hydrogen or C₁₋₆ alkyl.

In yet another aspect, R^{1d} is —OR^{d}, —SR^{d}, —NR^{d}₂, —C(O)R^{d}, —C(O)OR^{d}, —C(O)NR^{d}₂, —N(R^{d})C(O)R^{d}, —N(R^{d})C(O)OR^{d}, —N(R^{d})C(O)NR^{d}₂, phenyl, monocyclic heteroaryl, C₃₋₈ cycloalkyl, or monocyclic heterocyclyl, wherein the aryl, heteroaryl, C₃₋₈ cycloalkyl, and heterocyclyl groups are each optionally substituted by one, two, or three groups that are each independently halogen, cyano, nitro, C₁₋₆ alkyl, C₁₋₆ haloalkyl, —OR^{d}, —SR^{d}, —NR^{d}₂, —C(O)R^{d}, C(O)OR^{d}, —C(O)NR^{d}₂, —S(O)₂R^{d}, —OC(O)R^{d}, —OC(O)OR^{d}, OC(O)NR^{d}₂, N(R^{d})C(O)R^{d}, —N(R^{d})C(O)OR^{d}, or —N(R^{d})C(O)NR^{d}2.

### C. PHARMACEUTICAL COMPOSITIONS

In another aspect is provided a pharmaceutical composition including a pharmaceutically acceptable excipient and a compound, or pharmaceutically acceptable salt thereof, as described herein (e.g. formula I, formula II, formula III, aspect, embodiment, example, figure, table, or claim).

In embodiments of the pharmaceutical compositions, the compound, or pharmaceutically acceptable salt thereof, as described herein (e.g. formula I, formula II, formula III, aspect, embodiment, example, figure, table, or claim) is included in a therapeutically effective amount.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating cancer (e.g. multiple myeloma or cancers of secretory cells), neurodegenerative diseases, demyelinating diseases, eye diseases, fibrotic diseases, or diabetes. In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating a demyelinating disease. In embodiments, the second agent is an agent for treating an eye disease. In embodiments, the second agent is an agent for treating a fibrotic disease. In embodiments, the second agent is an agent for treating multiple sclerosis. In embodiments, the second agent is an agent for treating Alzheimer's disease. In embodiments, the second agent is an agent for treating Parkinson's disease. In embodiments, the second agent is an agent for treating Huntington's disease. In embodiments, the second agent is an agent for treating a prion disease. In embodiments, the second agent is an agent for treating amyotrophic lateral sclerosis. In embodiments, the second agent is an agent for treating diabetes. In embodiments, the second agent is an agent for treating retinal degeneration. In embodiments, the second agent is an agent for treating retinitis pigmentosa. In embodiments, the second agent is an agent for treating macular degeneration. In embodiments, the second agent is an agent for treating type I diabetes. In embodiments, the second agent is an agent for treating type II diabetes. In embodiments, the second agent is an agent for treating multiple myeloma. In embodiments, the second agent is an agent for treating a cancer of a secretory cell. In embodiments, the second agent is an agent for reducing Ire1 (e.g. Ire1α) kinase activity. In embodiments, the second agent is an agent for reducing Ire1 (e.g. Ire1α) RNase activity. In embodiments, the second agent is an agent for inhibiting a pathway activated by Ire1 (e.g. Ire1α) phosphorylation. In embodiments, the second agent is an agent for inhibiting a pathway activated by Ire1 (e.g. Ire1α) RNase activity. In embodiments, the second agent is an agent for inhibiting Ire1 (e.g. Ire1α) oligomerization. In embodiments, the second agent is an agent for inhibiting apoptosis. In embodiments, the second agent is in a synergistic amount. In embodiments, the compound described herein is in a synergistic amount. In embodiments, the compound described herein and the second agent are present in synergistic amounts. In embodiments, the compound described herein and the second agent are present in a combined synergistic amount.

In an aspect is provided a pharmaceutical combination including a first amount of an Ire1 kinase modulating compound and a second amount of an Ire1 ribonuclease modulating compound, wherein the first amount and the second amount are together a combined synergistic amount.

In embodiments, the Ire1 kinase modulating compound and the Ire1 ribonuclease modulating compound are in a single dosage form. In embodiments, the single dosage form further includes a pharmaceutically acceptable excipient. In embodiments, the Ire1 kinase modulating compound is in a first dosage form and the Ire1 ribonuclease modulating compound is in a second dosage form. In embodiments, the first dosage form further includes a first pharmaceutically acceptable excipient and the second dosage form further includes a second pharmaceutically acceptable excipient. In embodiments, the kinase modulating is kinase inhibiting. In embodiments, the ribonuclease modulating is ribonuclease inhibiting. In embodiments, the Ire1 kinase modulating compound is a compound described herein, including in an embodiment, example, figure, table, claim, or method (e.g., method of treatment or method of modulating Ire1). In embodiments, the Ire1 kinase modulating compound is in a synergistic amount. In embodiments, the Ire1 ribonuclease modulating compound is a derivative, analog, or prodrug of STF-083010, MKC-3946, 4μ8C, 3-methoxy-6-bromosalicylaldehyde, a salicylaldehyde, or toyocamycin. In embodiments, the Ire1 ribonuclease modulating compound is STF-083010, MKC-3946, 4μ8C, 3-methoxy-6-bromosalicylaldehyde, a salicylaldehyde, or toyocamycin. In embodiments, the Ire1 ribonuclease modulating compound is a derivative, analog, or prodrug of STF-083010. In embodiments, the Ire1 ribonuclease modulating compound is a derivative, analog, or prodrug of MKC-3946. In embodiments, the Ire1 ribonuclease modulating compound is a derivative, analog, or prodrug of 4μ8C. In embodiments, the Ire1 ribonuclease modulating compound is a derivative, analog, or prodrug of 3-methoxy-6-bromosalicylaldehyde. In embodiments, the Ire1 ribonuclease modulating compound is a derivative, analog, or prodrug of a salicylaldehyde. In embodiments, the Ire1 ribonuclease modulating compound is a derivative, analog, or prodrug of toyocamycin. In embodiments, the Ire1 ribonuclease modulating compound is STF-083010. In embodiments, the Ire1 ribonuclease modulating compound is MKC-3946. In embodiments, the Ire1 ribonuclease modulating compound is 4μ8C. In embodiments, the Ire1 ribonuclease modulating compound is 3-methoxy-6-bromosalicylaldehyde. In embodiments, the Ire1 ribonuclease modulating compound is a salicylaldehyde. In embodiments, the Ire1 ribonuclease modulating compound is toyocamycin. In embodiments, the Ire1 ribonuclease modulating compound is STF-083010. In embodiments, the Ire1 ribonuclease modulating compound is in a synergistic amount. In embodiments, Ire1 is Ire1α. In embodiments, the Ire1 ribonuclease modulating compound modulates XBP1 cleavage. In embodiments, the Ire1 ribonuclease modulating compound modulates XBP1 splicing. In embodiments, the Ire1 ribonuclease modulating compound modulates cleavage of an mRNA other than XBP1.

### D. METHODS

In an aspect is provided a method of treating a disease in a patient in need of such treatment, the method including administering a therapeutically effective amount of a compound described herein (e.g. formula I, formula II, formula III, aspect, embodiment, example, figure, table, or claim), or a pharmaceutically acceptable salt thereof, to the patient, wherein the disease is a neurodegenerative disease, demyelinating disease, cancer, eye disease, fibrotic disease, or diabetes.

In embodiments, the disease is a neurodegenerative disease, demyelinating disease, cancer, or diabetes. In embodiments, the disease is a neurodegenerative disease. In embodiments, the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru. In embodiments, the disease is amyotrophic lateral sclerosis. In embodiments, the disease is retinal degeneration. In embodiments, the disease is retinitis pigmentosa. In embodiments, the disease is a demyelinating disease. In embodiments, the demyelinating disease is Wolfram Syndrome, Pelizaeus-Merzbacher Disease, Transverse Myelitis, Charcot-Marie-Tooth Disease, or Multiple Sclerosis. In embodiments, the disease is Multiple Sclerosis. In embodiments, the disease is cancer. In embodiments, the cancer is multiple myeloma. In embodiments, the disease is diabetes. In embodiments, the diabetes is type I diabetes. In embodiments, the diabetes is type II diabetes. In embodiments, the disease is a neurodegenerative disease, demyelinating disease, cancer, eye disease, fibrotic disease, or diabetes described herein. In embodiments, the disease is an eye disease. In embodiments, the eye disease is retinitis pigmentosa. In embodiments, the eye disease is retinal degeneration. In embodiments, the eye disease is macular degeneration. In embodiments, the eye disease is Wolfram Syndrome. In embodiments, the disease is idiopathic pulmonary fibrosis (IPF). In embodiments, the disease is a fibrotic disease. In embodiments, the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis. In embodiments, the disease is interstitial lung disease (ILD). In embodiments, the disease is myocardial infarction. In embodiments, the disease is cardiac hypertrophy. In embodiments, the disease is heart failure. In embodiments, the disease is cirrhosis. In embodiments, the disease is acetominophen (Tylenol) liver toxicity. In embodiments, the disease is hepatitis C liver disease. In embodiments, the disease is hepatosteatosis (fatty liver disease). In embodiments, the disease is hepatic fibrosis.

In embodiments of the method, the method includes administering a second agent (e.g. therapeutic agent). In embodiments of the method, the method includes administering a second agent (e.g. therapeutic agent) in a therapeutically effective amount. In embodiments of the method, the second agent is an agent for treating cancer (e.g. multiple myeloma or cancers of secretory cells), neurodegenerative diseases, demyelinating diseases, eye diseases, fibrotic diseases, or diabetes. In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating a demyelinating disease. In embodiments, the second agent is an agent for treating an eye disease. In embodiments, the second agent is an agent for treating a fibrotic disease. In embodiments, the second agent is an agent for treating multiple sclerosis. In embodiments, the second agent is an agent for treating Alzheimer's disease. In embodiments, the second agent is an agent for treating Parkinson's disease. In embodiments, the second agent is an agent for treating Huntington's disease. In embodiments, the second agent is an agent for treating a prion disease. In embodiments, the second agent is an agent for treating amyotrophic lateral sclerosis. In embodiments, the second agent is an agent for treating diabetes. In embodiments, the second agent is an agent for treating retinal degeneration. In embodiments, the second agent is an agent for treating retinitis pigmentosa. In embodiments, the second agent is an agent for treating macular degeneration. In embodiments, the second agent is an agent for treating type I diabetes. In embodiments, the second agent is an agent for treating type II diabetes. In embodiments, the second agent is an agent for treating multiple myeloma. In embodiments, the second agent is an agent for treating a cancer of a secretory cell. In embodiments, the second agent is an agent for reducing Ire1 (e.g. Ire1α) kinase activity. In embodiments, the second agent is an agent for reducing Ire1 (e.g. Ire1α) RNase activity. In embodiments, the second agent is an agent for inhibiting a pathway activated by Ire1 (e.g. Ire1α) phosphorylation. In embodiments, the second agent is an agent for inhibiting a pathway activated by Ire1 (e.g. Ire1α) RNase activity. In embodiments, the second agent is an agent for inhibiting Ire1 (e.g. Ire1α) oligomerization. In embodiments, the second agent is an agent for inhibiting apoptosis. In embodiments, the second agent is administered in a synergistic amount. In embodiments, the compound described herein is administered in a synergistic amount. In embodiments, the compound described herein and the second agent are administered in synergistic amounts. In embodiments, the compound described herein and the second agent are present in a combined synergistic amount.

In an aspect is provided a method of modulating the activity of an Ire1 (e.g. Ire1α) protein, the method including contacting the Ire1 (e.g. Ire1α) protein with an effective amount of a compound described herein (e.g. formula I, formula II, formula III, aspect, embodiment, example, figure, table, or claim), or a pharmaceutically acceptable salt thereof.

In embodiments, the modulating is inhibiting. In embodiments, the activity is kinase activity. In embodiments, the kinase activity is autophosphorylation activity. In embodiments, the kinase activity is trans-autophosphorylation activity. In embodiments, the activity is oligomerization activity. In embodiments, the oligomerization activity is dimerization activity. In embodiments, the activity is RNase activity. In embodiments, the activity is miR-17 cleavage. In embodiments, the activity is miR-34a cleavage. In embodiments, the activity is miR-96 cleavage. In embodiments, the activity is miR-125b cleavage. In embodiments, the activity is XBP1 mRNA splicing. In embodiments, the activity is UPR activation. In embodiments, the activity is terminal UPR activation. In embodiments, a cell includes the Ire1 (e.g. Ire1α) protein. In embodiments, the activity of the Ire1 (e.g. Ire1α) protein is increasing apoptosis of the cell. In embodiments, an organ includes the cell. In embodiments, an organism includes the cell. In embodiments, an organism has a disease associated with the Ire1 (e.g. Ire1α) protein activity. In embodiments, the disease is a neurodegenerative disease, a demyelinating disease, cancer, an eye disease, a fibrotic disease, or diabetes. In embodiments, the disease is a neurodegenerative disease. In embodiments, the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru. In embodiments, the disease is amyotrophic lateral sclerosis. In embodiments, the disease is retinal degeneration. In embodiments, the disease is retinitis pigmentosa. In embodiments, the disease is a demyelinating disease. In embodiments, the demyelinating disease is Wolfram Syndrome, Pelizaeus-Merzbacher Disease, Transverse Myelitis, Charcot-Marie-Tooth Disease, or Multiple Sclerosis. In embodiments, the disease is Multiple Sclerosis. In embodiments, the disease is cancer. In embodiments, the cancer is multiple myeloma. In embodiments, the disease is diabetes. In embodiments, the diabetes is type I diabetes. In embodiments, the diabetes is type II diabetes. In embodiments, the disease is an eye disease. In embodiments, the eye disease is retinitis pigmentosa. In embodiments, the eye disease is retinal degeneration. In embodiments, the eye disease is macular degeneration. In embodiments, the eye disease is Wolfram Syndrome. In embodiments, the disease is idiopathic pulmonary fibrosis (IPF). In embodiments, the disease is a fibrotic disease. In embodiments, the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis. In embodiments, the disease is interstitial lung disease (ILD). In embodiments, the disease is myocardial infarction. In embodiments, the disease is cardiac hypertrophy. In embodiments, the disease is heart failure. In embodiments, the disease is cirrhosis. In embodiments, the disease is acetominophen (Tylenol) liver toxicity. In embodiments, the disease is hepatitis C liver disease. In embodiments, the disease is hepatosteatosis (fatty liver disease). In embodiments, the disease is hepatic fibrosis. In embodiments, the Ire1 protein is an Ire1α protein. In embodiments, the Ire1 (e.g. Ire1α) protein is a human protein. In embodiments, the Ire1 protein is a human Ire1α protein.

In embodiments of the method, the method includes contacting the Ire1 (e.g. Ire1α) protein with a second agent (e.g. therapeutic agent). In embodiments of the method, the method includes a second agent (e.g. therapeutic agent) in a therapeutically effective amount. In embodiments of the method, the second agent is an agent for treating cancer (e.g. multiple myeloma or cancers of secretory cells), neurodegenerative diseases, demyelinating diseases, eye diseases, fibrotic diseases, or diabetes. In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating a demyelinating disease. In embodiments, the second agent is an agent for treating an eye disease. In embodiments, the second agent is an agent for treating a fibrotic disease. In embodiments, the second agent is an agent for treating multiple sclerosis. In embodiments, the second agent is an agent for treating Alzheimer's disease. In embodiments, the second agent is an agent for treating Parkinson's disease. In embodiments, the second agent is an agent for treating Huntington's disease. In embodiments, the second agent is an agent for treating a prion disease. In embodiments, the second agent is an agent for treating amyotrophic lateral sclerosis. In embodiments, the second agent is an agent for treating diabetes. In embodiments, the second agent is an agent for treating retinal degeneration. In embodiments, the second agent is an agent for treating retinitis pigmentosa. In embodiments, the second agent is an agent for treating macular degeneration. In embodiments, the second agent is an agent for treating type I diabetes. In embodiments, the second agent is an agent for treating type II diabetes. In embodiments, the second agent is an agent for treating multiple myeloma. In embodiments, the second agent is an agent for treating a cancer of a secretory cell. In embodiments, the second agent is an agent for reducing Ire1 (e.g. Ire1α) kinase activity. In embodiments, the second agent is an agent for reducing Ire1 (e.g. Ire1α) RNase activity. In embodiments, the second agent is an agent for inhibiting a pathway activated by Ire1 (e.g. Ire1α) phosphorylation. In embodiments, the second agent is an agent for inhibiting a pathway activated by Ire1 (e.g. Ire1α) RNase activity. In embodiments, the second agent is an agent for inhibiting Ire1 (e.g. Ire1α) oligomerization. In embodiments, the second agent is an agent for inhibiting apoptosis. In embodiments, the second agent is administered in a synergistic amount. In embodiments, the compound described herein is administered in a synergistic amount. In embodiments, the compound described herein and the second agent are administered in synergistic amounts. In embodiments, the compound described herein and the second agent are administered in a combined synergistic amount. In embodiments, the second agent is bortezomib. In embodiments, the second agent is a proteasome inhibitor. In embodiments, the second agent is sunitinib.

In another aspect, the present disclosure, has identified two classes of kinase inhibitors-called types I and II, which stabilize alternate kinase active site conformations in numerous protein kinase targets (Liu, Y. & Gray, N. S. Nat. Chem. Biol. 2, 358-364 (2006)). The present disclosure shows that a type I kinase inhibitor and a novel type II kinase inhibitor both modify IRE1α by shutting down IRE1α trans-autophosphorylation, but have divergent effects on its RNase to activate or inactivate catalytic activity, respectively. The present disclosure further demonstrates that IRE1α RNase activity can be either up or downregulated through selective targeting of its kinase domain to control UPR signaling, and predict that it may be possible to pharmacologically modulate other kinase-coupled enzymes in a similar way.

In an additional aspect, the present disclosure illustrates that IRE1α's kinase-controlled RNase can be regulated in two distinct modes with kinase inhibitors: one class of ligands occupy IRE1α's kinase ATP-binding site to activate RNase-mediated XBP1 mRNA splicing even without upstream ER stress, while a second class can inhibit the RNase through the same ATP-binding site, even under ER stress. Thus, alternative kinase conformations stabilized by distinct classes of ATP-competitive inhibitors can cause allosteric switching of IRE1α's RNase—either on or off. As dysregulation of the UPR has been implicated in a variety of cell degenerative and neoplastic disorders, small molecule control over IRE1α should advance efforts to understand the UPR's role in pathophysiology and to develop drugs for ER stress-related diseases.

### E. COMBINATION METHODS AND USES

In an aspect is provided a method of treating a disease in a patient in need of such treatment, the method including administering a first amount of an Ire1 kinase modulating compound, or a pharmaceutically acceptable salt thereof, and a second amount of an Ire1 ribonuclease modulating compound, or a pharmaceutically acceptable salt thereof, to the patient, wherein the disease is a neurodegenerative disease, demyelinating disease, cancer, eye disease, fibrotic disease, or diabetes, wherein the first amount and the second amount are together a combined synergistic amount.

In an aspect is provided a method of treating a disease in a patient in need of such treatment, the method including administering a first amount of an Ire1 kinase modulating compound, or a pharmaceutically acceptable salt thereof, and a second amount of an Ire1 ribonuclease modulating compound, or a pharmaceutically acceptable salt thereof, to the patient, wherein the disease is associated with Ire1 (e.g., Ire1α) activity (e.g., RNase activity, kinase activity, or oligomerization).

In an aspect is provided a pharmaceutical composition comprising a pharmaceutically acceptable excipient; a first amount of an Ire1 kinase modulating compound, or a pharmaceutically acceptable salt thereof; and a second amount of an Ire1 ribonuclease modulating compound, or a pharmaceutically acceptable salt thereof; for use in treating a disease in a subject in need of such treatment, wherein the disease is a neurodegenerative disease, demyelinating disease, cancer, eye disease, fibrotic disease, or diabetes, wherein the first amount and the second amount are together a combined synergistic amount.

In an aspect is provided a pharmaceutical composition comprising a pharmaceutically acceptable excipient; a first amount of an Ire1 kinase modulating compound, or a pharmaceutically acceptable salt thereof, and a second amount of an Ire1 ribonuclease modulating compound, or a pharmaceutically acceptable salt thereof, for use in treating a disease in a subject in need of such treatment, wherein the disease is associated with Ire1 (e.g., Ire1α) activity (e.g., RNase activity, kinase activity, or oligomerization).

In another aspect is provided a pharmaceutical composition described herein, including embodiments, for use as a medicament.

In another aspect is provided the use of a pharmaceutical composition described herein, including embodiments, in the manufacture of a medicament for the treatment of a disease in a subject in need of such treatment. The disease may be associated with Ire1 (e.g., Ire1α) activity (e.g., RNase activity, kinase activity, or oligomerization). The disease may be a neurodegenerative disease, demyelinating disease, cancer, eye disease, fibrotic disease, or diabetes.

In embodiments, the disease is a neurodegenerative disease, demyelinating disease, cancer, or diabetes. In embodiments, the disease is a neurodegenerative disease. In embodiments, the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru. In embodiments, the disease is a demyelinating disease. In embodiments, the demyelinating disease is Wolfram Syndrome, Pelizaeus-Merzbacher Disease, Transverse Myelitis, Charcot-Marie-Tooth Disease, or Multiple Sclerosis. In embodiments, the disease is cancer. In embodiments, the cancer is multiple myeloma. In embodiments, the disease is diabetes. In embodiments, the diabetes is type I diabetes. In embodiments, the diabetes is type II diabetes. In embodiments, the disease is an eye disease. In embodiments, the eye disease is retinitis pigmentosa, retinal degeneration, macular degeneration, or Wolfram Syndrome. In embodiments, the disease is a fibrotic disease. In embodiments, the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

In embodiments, the Ire1 kinase modulating compound is a compound (e.g., KIRA) described herein (e.g., in an aspect, embodiment, compound section, example, claim, figure, table). In embodiments, the Ire1 kinase modulating compound has the formula:

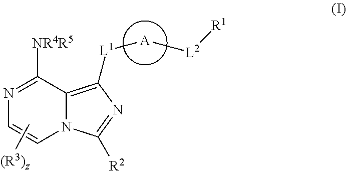

wherein Ring A, L¹, L², R¹, R², R³, R⁴, R⁵, and z are as described herein (including above, in the compound section, in an aspect, embodiment, figures, table, example, or claim).

In embodiments, ring A is substituted or unsubstituted cycloalkylene, substituted or unsubstituted heterocycloalkylene, substituted or unsubstituted arylene, or substituted or unsubstituted heteroarylene; L¹ is a bond or unsubstituted C₁-C₅ alkylene; L² is a bond, —NR^{6a}—, —O—, —S—, —C(O)—, —S(O)—, —S(O)₂—, —NR^{6a}C(O)—, —C(O)NR^{6b}—, —C(O)(CH₂)_{z2}—, —NR^{6a}C(O)O—, —NR^{6a}C(O)NR^{6b}-substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹ is hydrogen, oxo, halogen, —CX₃, —CN, —SO₂Cl, —SOᵥR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, oxo, halogen, —CX^{a}₃, —CN, —SO₂Cl, —SOᵥ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SO₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, oxo, halogen, —CX^{b}₃, —CN, —SO₂Cl, —SO₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, —NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ and R⁵ are independently hydrogen or unsubstituted C₁-C₆ alkyl; R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl; each occurrence of the symbols n, n1, and n2 is independently an integer from 0 to 4; each occurrence of the symbols m, m1, m2, v, v1, and v2 is independently an integer from 1 to 2; the symbol z is an integer from 0 to 2; the symbol z2 is an integer from 1 to 4; each occurrence of the symbols X, X^{a}, and X^{b} is independently a halogen.

In embodiments, R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is hydrogen. In embodiments, the symbol z is 1. In embodiments, R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R² is substituted or unsubstituted alkyl. In embodiments, R² is substituted or unsubstituted C₁-C₆ alkyl. In embodiments, R² is unsubstituted C₁-C₆ alkyl. In embodiments, R² is unsubstituted isopropyl. In embodiments, R² is unsubstituted tert-butyl. In embodiments, R⁴ and R⁵ are hydrogen. In embodiments, L¹ is a bond. In embodiments, L¹ is unsubstituted methylene. In embodiments, L² is —NR^{6a}C(O)NR^{6b}—. In embodiments, R^{6a} and R^{6b} are hydrogen. In embodiments, R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R¹ is substituted or unsubstituted cycloalkyl, substituted or unsubstituted heterocycloalkyl, substituted or unsubstituted aryl, or substituted or unsubstituted heteroaryl. In embodiments, R¹ is substituted or unsubstituted aryl or substituted or unsubstituted heteroaryl. In embodiments, R¹ is substituted phenyl. In embodiments, R¹ is phenyl substituted with —CF₃ or halogen. In embodiments, ring A is substituted or unsubstituted arylene or substituted or unsubstituted heteroarylene. In embodiments, ring A is substituted or unsubstituted C₆-C₁₀ arylene. In embodiments, ring A is unsubstituted naphthalenyl.

In embodiments, the Ire1 kinase modulating compound has the formula:

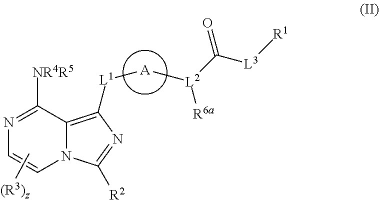

wherein Ring A, L¹, L³, R¹, R², R³, R⁴, R⁵, R^{6a}, and z are as described herein (including above, in the compound section, in an aspect, embodiment, figures, table, example, or claim).

In embodiments, L³ is a bond, —NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, the Ire1 kinase modulating compound has the formula:

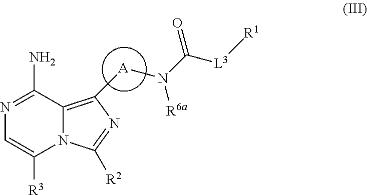

wherein Ring A, L³, R¹, R², R³, and R^{6a} are as described herein (including above, in the compound section, in an aspect, embodiment, figures, table, example, or claim). In embodiments, the Ire1 kinase modulating compound is selected from the group consisting of:

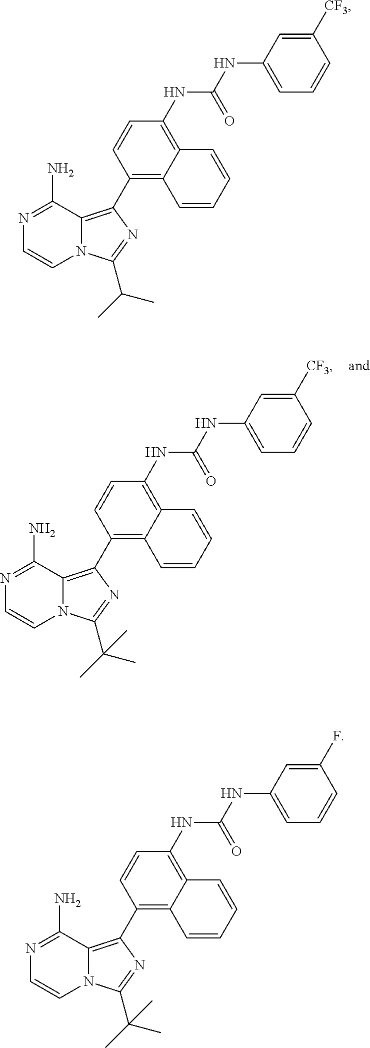 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "and" ; CC(C)(C)c1nc(-c2ccc(NC(=O)Nc3cccc(F)c3)c3ccccc23)c2c(N)nccn12]

In embodiments, the Ire1 kinase modulating compound is

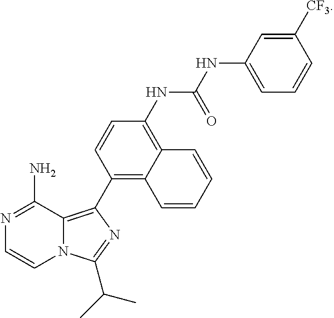 [SMILES: CC(C)c1nc(-c2ccc(NC(=O)Nc3cccc(C(F)(F)F)c3)c3ccccc23)c2c(N)nccn12]

In embodiments, the Ire1 kinase modulating compound is

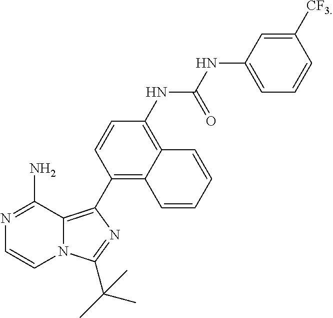 [SMILES: CC(C)(C)c1nc(-c2ccc(NC(=O)Nc3cccc(C(F)(F)F)c3)c3ccccc23)c2c(N)nccn12]

In embodiments, the Ire1 kinase modulating compound

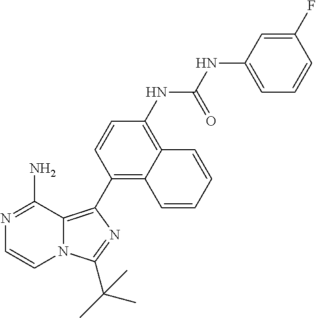 [SMILES: CC(C)(C)c1nc(-c2ccc(NC(=O)Nc3cccc(F)c3)c3ccccc23)c2c(N)nccn12]

In embodiments, the Ire1 kinase modulating compound is a compound described herein, including in embodiments, an example, table, figure, appendix, or claim. In embodiments, the activity of the Ire1 protein is XBP1 cleavage. In embodiments, the activity of the Ire1 protein is XBP1 splicing. In embodiments, the activity of the Ire1 protein is cleavage of an mRNA other than XBP1.

In embodiments, the Ire1 kinase modulating compound is administered in a synergistic amount. In embodiments, the Ire1 ribonuclease modulating compound is administered in a synergistic amount. In embodiments, the Ire1 ribonuclease modulating compound is a derivative, analog, or prodrug of STF-083010, MKC-3946, 4μ8C, 3-methoxy-6-bromosalicylaldehyde, a salicylaldehyde, or toyocamycin. In embodiments, the Ire1 ribonuclease modulating compound is STF-083010, MKC-3946, 4μ8C, 3-methoxy-6-bromosalicylaldehyde, a salicylaldehyde, or toyocamycin. In embodiments, the Ire1 ribonuclease modulating compound is STF-083010. In embodiments, the Ire1 ribonuclease modulating compound is administered in a synergistic amount. In embodiments, Ire1 is Ire1α. In embodiments, the Ire1 ribonuclease modulating compound is a derivative, analog, or prodrug of STF-083010. In embodiments, the Ire1 ribonuclease modulating compound is a derivative, analog, or prodrug of MKC-3946. In embodiments, the Ire1 ribonuclease modulating compound is a derivative, analog, or prodrug of 4μ8C. In embodiments, the Ire1 ribonuclease modulating compound is a derivative, analog, or prodrug of 3-methoxy-6-bromosalicylaldehyde. In embodiments, the Ire1 ribonuclease modulating compound is a derivative, analog, or prodrug of a salicylaldehyde. In embodiments, the Ire1 ribonuclease modulating compound is a derivative, analog, or prodrug of toyocamycin. In embodiments, the Ire1 ribonuclease modulating compound is STF-083010. In embodiments, the Ire1 ribonuclease modulating compound is MKC-3946. In embodiments, the Ire1 ribonuclease modulating compound is 4μ8C. In embodiments, the Ire1 ribonuclease modulating compound is 3-methoxy-6-bromosalicylaldehyde. In embodiments, the Ire1 ribonuclease modulating compound is a salicylaldehyde. In embodiments, the Ire1 ribonuclease modulating compound is toyocamycin.

In an aspect is provided a method of modulating the activity of an Ire1 protein, the method including contacting the Ire1 protein with an Ire1 kinase modulating compound, or a pharmaceutically acceptable salt thereof, and an Ire1 ribonuclease modulating compound, or a pharmaceutically acceptable salt thereof.

In an aspect is provided a pharmaceutical composition comprising a pharmaceutically acceptable excipient; a first amount of an Ire1 kinase modulating compound, or a pharmaceutically acceptable salt thereof, and a second amount of an Ire1 ribonuclease modulating compound, or a pharmaceutically acceptable salt thereof, for use in modulating the activity of an Ire1 protein, the method including contacting the Ire1 protein with the Ire1 kinase modulating compound and the Ire1 ribonuclease modulating compound.

In embodiments, the kinase modulating is kinase inhibiting. In embodiments, the ribonuclease modulating is ribonuclease inhibiting. In embodiments, the activity of an Ire1 protein is kinase activity. In embodiments, the kinase activity is autophosphorylation activity. In embodiments, the activity of an Ire1 protein is oligomerization activity. In embodiments, the oligomerization activity is dimerization activity. In embodiments, the activity of an Ire1 protein is RNase activity. In embodiments, a cell includes the Ire1 protein. In embodiments, the activity of the Ire1 protein is increasing apoptosis of the cell. In embodiments, an organ includes the cell. In embodiments, an organism includes the cell. In embodiments, the organism has a disease associated with the Ire1 protein activity. In embodiments, the disease is a neurodegenerative disease, demyelinating disease, cancer, eye disease, fibrotic disease, or diabetes. In embodiments, the disease is a neurodegenerative disease. In embodiments, the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru. In embodiments, the disease is a demyelinating disease. In embodiments, the demyelinating disease is Wolfram Syndrome, Pelizaeus-Merzbacher Disease, Transverse Myelitis, Charcot-Marie-Tooth Disease, or Multiple Sclerosis. In embodiments, the demyelinating disease is Multiple Sclerosis. In embodiments, the disease is cancer. In embodiments, the cancer is multiple myeloma. In embodiments, the disease is diabetes. In embodiments, the diabetes is type I diabetes. In embodiments, the diabetes is type II diabetes. In embodiments, the disease is an eye disease. In embodiments, the eye disease is retinitis pigmentosa, retinal degeneration, macular degeneration, or Wolfram Syndrome. In embodiments, the disease is a fibrotic disease. In embodiments, the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

In embodiments, the Ire1 kinase modulating compound is a compound described herein, including in an embodiment, example, figure, table, claim, or different method (e.g., method of treatment). In embodiments, the Ire1 kinase modulating compound is administered in a synergistic amount. In embodiments, the Ire1 ribonuclease modulating compound is an Ire1 ribonuclease modulating compound described herein (e.g., above). In embodiments, the Ire1 ribonuclease modulating compound is a derivative, analog, or prodrug of STF-083010, MKC-3946, 4μ8C, 3-methoxy-6-bromosalicylaldehyde, a salicylaldehyde, or toyocamycin. In embodiments, the Ire1 ribonuclease modulating compound is STF-083010, MKC-3946, 4μ8C, 3-methoxy-6-bromosalicylaldehyde, a salicylaldehyde, or toyocamycin. In embodiments, the Ire1 ribonuclease modulating compound is STF-083010. In embodiments, the Ire1 ribonuclease modulating compound is administered in a synergistic amount. In embodiments, the Ire1 kinase modulating compound and the Ire1 ribonuclease modulating compound are administered in a combined synergistic amount. In embodiments, Ire1 is Ire1α. In embodiments, the activity of the Ire1 protein is XBP1 cleavage. In embodiments, the activity of the Ire1 protein is XBP1 splicing. In embodiments, the activity of the Ire1 protein is cleavage of an mRNA other than XBP1.

In embodiments (e.g., of the methods, uses, or pharmaceutical compositions), the Ire1 kinase modulating compound is KIRA3 and the Ire1 ribonuclease modulating compound is STF-083010. In embodiments (e.g., of the methods, uses, or pharmaceutical compositions), the Ire1 kinase modulating compound is KIRA3 and the Ire1 ribonuclease modulating compound is MKC-3946. In embodiments (e.g., of the methods, uses, or pharmaceutical compositions), the Ire1 kinase modulating compound is KIRA3 and the Ire1 ribonuclease modulating compound is 4μ8C. In embodiments (e.g., of the methods, uses, or pharmaceutical compositions), the Ire1 kinase modulating compound is KIRA3 and the Ire1 ribonuclease modulating compound is 3-methoxy-6-bromosalicylaldehyde. In embodiments (e.g., of the methods, uses, or pharmaceutical compositions), the Ire1 kinase modulating compound is KIRA3 and the Ire1 ribonuclease modulating compound is a salicylaldehyde. In embodiments (e.g., of the methods, uses, or pharmaceutical compositions), the Ire1 kinase modulating compound is KIRA3 and the Ire1 ribonuclease modulating compound is toyocamycin. In embodiments (e.g., of the methods, uses, or pharmaceutical compositions), the Ire1 kinase modulating compound is KIRA3 and the Ire1 ribonuclease modulating compound is a derivative, analog, or prodrug of STF-083010. In embodiments (e.g., of the methods, uses, or pharmaceutical compositions), the Ire1 kinase modulating compound is KIRA3 and the Ire1 ribonuclease modulating compound is a derivative, analog, or prodrug of MKC-3946. In embodiments (e.g., of the methods, uses, or pharmaceutical compositions), the Ire1 kinase modulating compound is KIRA3 and the Ire1 ribonuclease modulating compound is a derivative, analog, or prodrug of 4μ8C. In embodiments (e.g., of the methods, uses, or pharmaceutical compositions), the Ire1 kinase modulating compound is KIRA3 and the Ire1 ribonuclease modulating compound is a derivative, analog, or prodrug of 3-methoxy-6-bromosalicylaldehyde. In embodiments (e.g., of the methods, uses, or pharmaceutical compositions), the Ire1 kinase modulating compound is KIRA3 and the Ire1 ribonuclease modulating compound is a derivative, analog, or prodrug of a salicylaldehyde. In embodiments (e.g., of the methods, uses, or pharmaceutical compositions), the Ire1 kinase modulating compound is KIRA3 and the Ire1 ribonuclease modulating compound is a derivative, analog, or prodrug of toyocamycin.

In embodiments (e.g., of the methods, uses, or pharmaceutical compositions), the Ire1 kinase modulating compound is KIRA6 and the Ire1 ribonuclease modulating compound is STF-083010. In embodiments (e.g., of the methods, uses, or pharmaceutical compositions), the Ire1 kinase modulating compound is KIRA6 and the Ire1 ribonuclease modulating compound is MKC-3946. In embodiments (e.g., of the methods, uses, or pharmaceutical compositions), the Ire1 kinase modulating compound is KIRA6 and the Ire1 ribonuclease modulating compound is 4μ8C. In embodiments (e.g., of the methods, uses, or pharmaceutical compositions), the Ire1 kinase modulating compound is KIRA6 and the Ire1 ribonuclease modulating compound is 3-methoxy-6-bromosalicylaldehyde. In embodiments (e.g., of the methods, uses, or pharmaceutical compositions), the Ire1 kinase modulating compound is KIRA6 and the Ire1 ribonuclease modulating compound is a salicylaldehyde. In embodiments (e.g., of the methods, uses, or pharmaceutical compositions), the Ire1 kinase modulating compound is KIRA6 and the Ire1 ribonuclease modulating compound is toyocamycin. In embodiments (e.g., of the methods, uses, or pharmaceutical compositions), the Ire1 kinase modulating compound is KIRA6 and the Ire1 ribonuclease modulating compound is a derivative, analog, or prodrug of STF-083010. In embodiments (e.g., of the methods, uses, or pharmaceutical compositions), the Ire1 kinase modulating compound is KIRA6 and the Ire1 ribonuclease modulating compound is a derivative, analog, or prodrug of MKC-3946. In embodiments (e.g., of the methods, uses, or pharmaceutical compositions), the Ire1 kinase modulating compound is KIRA6 and the Ire1 ribonuclease modulating compound is a derivative, analog, or prodrug of 4μ8C. In embodiments (e.g., of the methods, uses, or pharmaceutical compositions), the Ire1 kinase modulating compound is KIRA6 and the Ire1 ribonuclease modulating compound is a derivative, analog, or prodrug of 3-methoxy-6-bromosalicylaldehyde. In embodiments (e.g., of the methods, uses, or pharmaceutical compositions), the Ire1 kinase modulating compound is KIRA6 and the Ire1 ribonuclease modulating compound is a derivative, analog, or prodrug of a salicylaldehyde. In embodiments (e.g., of the methods, uses, or pharmaceutical compositions), the Ire1 kinase modulating compound is KIRA6 and the Ire1 ribonuclease modulating compound is a derivative, analog, or prodrug of toyocamycin.

In embodiments (e.g., of the methods, uses, or pharmaceutical compositions), the Ire1 kinase modulating compound is KIRA7 and the Ire1 ribonuclease modulating compound is STF-083010. In embodiments (e.g., of the methods, uses, or pharmaceutical compositions), the Ire1 kinase modulating compound is KIRA7 and the Ire1 ribonuclease modulating compound is MKC-3946. In embodiments (e.g., of the methods, uses, or pharmaceutical compositions), the Ire1 kinase modulating compound is KIRA7 and the Ire1 ribonuclease modulating compound is 4μ8C. In embodiments (e.g., of the methods, uses, or pharmaceutical compositions), the Ire1 kinase modulating compound is KIRA7 and the Ire1 ribonuclease modulating compound is 3-methoxy-6-bromosalicylaldehyde. In embodiments (e.g., of the methods, uses, or pharmaceutical compositions), the Ire1 kinase modulating compound is KIRA7 and the Ire1 ribonuclease modulating compound is a salicylaldehyde. In embodiments (e.g., of the methods, uses, or pharmaceutical compositions), the Ire1 kinase modulating compound is KIRA7 and the Ire1 ribonuclease modulating compound is toyocamycin. In embodiments (e.g., of the methods, uses, or pharmaceutical compositions), the Ire1 kinase modulating compound is KIRA7 and the Ire1 ribonuclease modulating compound is a derivative, analog, or prodrug of STF-083010. In embodiments (e.g., of the methods, uses, or pharmaceutical compositions), the Ire1 kinase modulating compound is KIRA7 and the Ire1 ribonuclease modulating compound is a derivative, analog, or prodrug of MKC-3946. In embodiments (e.g., of the methods, uses, or pharmaceutical compositions), the Ire1 kinase modulating compound is KIRA7 and the Ire1 ribonuclease modulating compound is a derivative, analog, or prodrug of 4μ8C. In embodiments (e.g., of the methods, uses, or pharmaceutical compositions), the Ire1 kinase modulating compound is KIRA7 and the Ire1 ribonuclease modulating compound is a derivative, analog, or prodrug of 3-methoxy-6-bromosalicylaldehyde. In embodiments (e.g., of the methods, uses, or pharmaceutical compositions), the Ire1 kinase modulating compound is KIRA7 and the Ire1 ribonuclease modulating compound is a derivative, analog, or prodrug of a salicylaldehyde. In embodiments (e.g., of the methods, uses, or pharmaceutical compositions), the Ire1 kinase modulating compound is KIRA7 and the Ire1 ribonuclease modulating compound is a derivative, analog, or prodrug of toyocamycin.

### F. ADDITIONAL EMBODIMENTS

1. A method of treating a disease in a patient in need of such treatment, said method comprising administering a first amount of an Ire1 kinase modulating compound, or a pharmaceutically acceptable salt thereof, and a second amount of an Ire1 ribonuclease modulating compound, or a pharmaceutically acceptable salt thereof, to said patient, wherein the disease is a neurodegenerative disease, demyelinating disease, cancer, eye disease, fibrotic disease, or diabetes, wherein the first amount and the second amount are together a combined synergistic amount.
2. The method of embodiment 1, wherein the disease is a neurodegenerative disease, demyelinating disease, cancer, or diabetes.
3. The method of embodiment 1, wherein the disease is a neurodegenerative disease.
4. The method of embodiment 3, wherein the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru.
5. The method of embodiment 1, wherein the disease is a demyelinating disease.
6. The method of embodiment 5, wherein the demyelinating disease is Wolfram Syndrome, Pelizaeus-Merzbacher Disease, Transverse Myelitis, Charcot-Marie-Tooth Disease, or Multiple Sclerosis.
7. The method of embodiment 1, wherein the disease is cancer.
8. The method of embodiment 7, wherein the cancer is multiple myeloma.
9. The method of embodiment 1, wherein the disease is diabetes.
10. The method of embodiment 9, wherein the diabetes is type I diabetes.
11. The method of embodiment 9, wherein the diabetes is type II diabetes.
12. The method of embodiment 1, wherein the disease is an eye disease.
13. The method of embodiment 12, wherein the eye disease is retinitis pigmentosa, retinal degeneration, macular degeneration, or Wolfram Syndrome.
14. The method of embodiment 1, wherein the disease is a fibrotic disease.
15. The method of embodiment 14, wherein the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.
16. The method of one of embodiments 1 to 15, wherein said Ire1 kinase modulating compound has the formula:

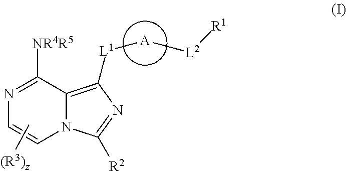

wherein, ring A is substituted or unsubstituted cycloalkylene, substituted or unsubstituted heterocycloalkylene, substituted or unsubstituted arylene, or substituted or unsubstituted heteroarylene; L¹ is a bond or unsubstituted C₁-C₅ alkylene; L² is a bond, —NR^{6a}—, —O—, —S—, —C(O)—, —S(O)—, —S(O)₂—, —NR^{6a}C(O)—, —C(O)NR^{6b}—, —C(O)(CH₂)_{z2}—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, oxo, halogen, —CX^{b}₃, —CN, —SO₂Cl, —SOₙ₂R^{1b}, —SO₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ and R⁵ are independently hydrogen or unsubstituted C₁-C₆ alkyl; R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl; each occurrence of the symbols n, n1, and n2 is independently an integer from 0 to 4; each occurrence of the symbols m, m1, m2, v, v1, and v2 is independently an integer from 1 to 2; the symbol z is an integer from 0 to 2; the symbol z2 is an integer from 1 to 4; each occurrence of the symbols X, X^{a}, and X^{b} is independently a halogen.
17. The method of embodiment 16, wherein R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
18. The method of embodiment 16, wherein R³ is hydrogen.
19. The method of one of embodiments 16 to 18, wherein the symbol z is 1.
20. The method of one of embodiments 16 to 19, wherein R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
21. The method of one of embodiments 16 to 19, wherein R² is substituted or unsubstituted alkyl.
22. The method of one of embodiments 16 to 19, wherein R² is substituted or unsubstituted C₁-C₆ alkyl.
23. The method of one of embodiments 16 to 19, wherein R² is unsubstituted C₁-C₆ alkyl.
24. The method of one of embodiments 16 to 19, wherein R² is unsubstituted isopropyl.
25. The method of one of embodiments 16 to 19, wherein R² is unsubstituted tert-butyl.
26. The method of one of embodiments 16 to 25, wherein R⁴ and R⁵ are hydrogen.
27. The method of one of embodiments 16 to 26, wherein L¹ is a bond.
28. The method of one of embodiments 16 to 26, wherein L¹ is unsubstituted methylene.
29. The method of one of embodiments 16 to 28, wherein L² is —NR^{6a}C(O)NR^{6b}—.
30. The method of embodiment 29, wherein R^{6a} and R^{6b} are hydrogen.
31. The method of one of embodiments 16 to 30, wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
32. The method of one of embodiments 16 to 30, wherein R¹ is substituted or unsubstituted cycloalkyl, substituted or unsubstituted heterocycloalkyl, substituted or unsubstituted aryl, or substituted or unsubstituted heteroaryl.
33. The method of one of embodiments 16 to 30, wherein R¹ is substituted or unsubstituted aryl or substituted or unsubstituted heteroaryl.
34. The method of one of embodiments 16 to 30, wherein R¹ is substituted phenyl.
35. The method of one of embodiments 16 to 30, wherein R¹ is phenyl substituted with —CF₃ or halogen.
36. The method of one of embodiments 16 to 35, wherein ring A is substituted or unsubstituted arylene or substituted or unsubstituted heteroarylene.
37. The method of one of embodiments 16 to 35, wherein ring A is substituted or unsubstituted C₆-C₁₀ arylene.
38. The method of one of embodiments 16 to 35, wherein ring A is unsubstituted naphthalenyl.
39. The method of one of embodiments 1 to 38, wherein the Ire1 kinase modulating compound has the formula:

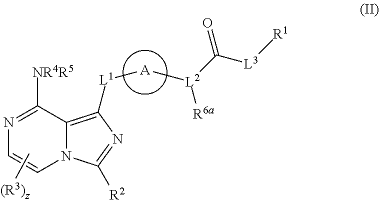

wherein, L³ is a bond, —NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
40. The method of embodiment 39, wherein the Ire1 kinase modulating compound has the formula:

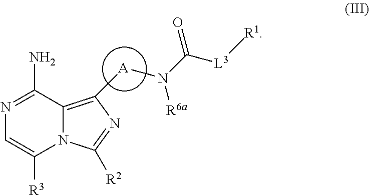

41. The method of embodiment 1, wherein the Ire1 kinase modulating compound is selected from the group consisting of:

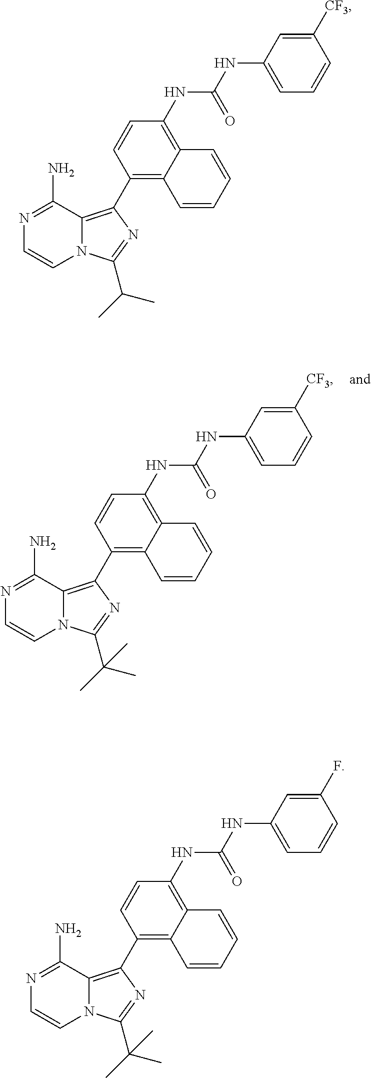 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "and" ; CC(C)(C)c1nc(-c2ccc(NC(=O)Nc3cccc(F)c3)c3ccccc23)c2c(N)nccn12]

42. The method of one of embodiments 1 to 41, wherein said Ire1 kinase modulating compound is administered in a synergistic amount.
43. The method of one of embodiments 1 to 42, wherein said Ire1 ribonuclease modulating compound is a derivative, analog, or prodrug of STF-083010, MKC-3946, 4μ8C, 3-methoxy-6-bromosalicylaldehyde, a salicylaldehyde, or toyocamycin.
44. The method of one of embodiments 1 to 42, wherein said Ire1 ribonuclease modulating compound is STF-083010, MKC-3946, 4μ8C, 3-methoxy-6-bromosalicylaldehyde, a salicylaldehyde, or toyocamycin.
45. The method of one of embodiments 1 to 42, wherein said Ire1 ribonuclease modulating compound is STF-083010.
46. The method of one of embodiments 1 to 45, wherein said Ire1 ribonuclease modulating compound is administered in a synergistic amount.
47. A method of modulating the activity of an Ire1 protein, said method comprising contacting said Ire1 protein with an Ire1 kinase modulating compound, or a pharmaceutically acceptable salt thereof, and an Ire1 ribonuclease modulating compound, or a pharmaceutically acceptable salt thereof.
48. The method of embodiment 47, wherein said kinase modulating is kinase inhibiting.
49. The method of embodiment 47, wherein said ribonuclease modulating is ribonuclease inhibiting.
50. The method of one of embodiments 47 to 49, wherein said activity of an Ire1 protein is kinase activity.
51. The method of embodiment 50, wherein said kinase activity is autophosphorylation activity.
52. The method of one of embodiments 47 to 49, wherein said activity of an Ire1 protein is oligomerization activity.
53. The method of embodiment 52, wherein said oligomerization activity is dimerization activity.
54. The method of one of embodiments 47 to 49, wherein said activity of an Ire1 protein is RNase activity.
55. The method of one of embodiments 47 to 54, wherein a cell comprises said Ire1 protein.
56. The method of embodiment 55, wherein said activity of the Ire1 protein is increasing apoptosis of said cell.
57. The method of one of embodiments 55 to 56, wherein an organ comprises said cell.
58. The method of one of embodiments 55 to 56, wherein an organism comprises said cell.
59. The method of embodiment 58, wherein said organism has a disease associated with said Ire1 protein activity.
60. The method of embodiment 59, wherein said disease is a neurodegenerative disease, demyelinating disease, cancer, eye disease, fibrotic disease, or diabetes
61. The method of embodiment 60, wherein the disease is a neurodegenerative disease.
62. The method of embodiment 61, wherein the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru.
63. The method of embodiment 60, wherein the disease is a demyelinating disease.
64. The method of embodiment 63, wherein the demyelinating disease is Wolfram Syndrome, Pelizaeus-Merzbacher Disease, Transverse Myelitis, Charcot-Marie-Tooth Disease, or Multiple Sclerosis.
65. The method of embodiment 63, wherein the demyelinating disease is Multiple Sclerosis.
66. The method of embodiment 60, wherein the disease is cancer.
67. The method of embodiment 66, wherein the cancer is multiple myeloma.
68. The method of embodiment 60, wherein the disease is diabetes.
69. The method of embodiment 68, wherein the diabetes is type I diabetes.
70. The method of embodiment 68, wherein the diabetes is type II diabetes.
71. The method of embodiment 60, wherein the disease is an eye disease.
72. The method of embodiment 71, wherein the eye disease is retinitis pigmentosa, retinal degeneration, macular degeneration, or Wolfram Syndrome.
73. The method of embodiment 60, wherein the disease is a fibrotic disease.
74. The method of embodiment 73, wherein the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.
75. The method of one of embodiments 47 to 74, wherein said Ire1 kinase modulating compound has the formula:

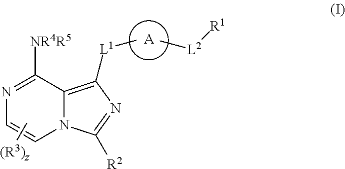

wherein, ring A is substituted or unsubstituted cycloalkylene, substituted or unsubstituted heterocycloalkylene, substituted or unsubstituted arylene, or substituted or unsubstituted heteroarylene; L¹ is a bond or unsubstituted C₁-C₅ alkylene; L² is a bond, —NR^{6a}—, —O—, —S—, —C(O)—, —S(O)—, —S(O)₂—, —NR^{6a}C(O)—, —C(O)NR^{6b}—, —C(O)(CH₂)_{z2}—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}—, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, oxo, halogen, —CX^{b}₃, —CN, —SO₂Cl, —SO₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, —NR^{7b}SO₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ and R⁵ are independently hydrogen or unsubstituted C₁-C₆ alkyl; R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl; each occurrence of the symbols n, n1, and n2 is independently an integer from 0 to 4; each occurrence of the symbols m, m1, m2, v, v1, and v2 is independently an integer from 1 to 2; the symbol z is an integer from 0 to 2; the symbol z2 is an integer from 1 to 4; each occurrence of the symbols X, X^{a}, and X^{b} is independently a halogen.
76. The method of embodiment 75, wherein R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
77. The method of embodiment 75, wherein R³ is hydrogen.
78. The method of one of embodiments 75 to 77, wherein the symbol z is 1.
79. The method of one of embodiments 75 to 78, wherein R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
80. The method of one of embodiments 75 to 78, wherein R² is substituted or unsubstituted alkyl.
81. The method of one of embodiments 75 to 78, wherein R² is substituted or unsubstituted C₁-C₆ alkyl.
82. The method of one of embodiments 75 to 78, wherein R² is unsubstituted C₁-C₆ alkyl.
83. The method of one of embodiments 75 to 78, wherein R² is unsubstituted isopropyl.
84. The method of one of embodiments 75 to 78, wherein R² is unsubstituted tert-butyl.
85. The method of one of embodiments 75 to 84, wherein R⁴ and R⁵ are hydrogen.
86. The method of one of embodiments 75 to 85, wherein L¹ is a bond.
87. The method of one of embodiments 75 to 85, wherein L¹ is unsubstituted methylene.
88. The method of one of embodiments 75 to 87, wherein L² is —NR^{6a}C(O)NR^{6b}—.
89. The method of embodiment 88, wherein R^{6a} and R^{6b} are hydrogen.
90. The method of one of embodiments 75 to 89, wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
91. The method of one of embodiments 75 to 89, wherein R¹ is substituted or unsubstituted cycloalkyl, substituted or unsubstituted heterocycloalkyl, substituted or unsubstituted aryl, or substituted or unsubstituted heteroaryl.
92. The method of one of embodiments 75 to 89, wherein R¹ is substituted or unsubstituted aryl or substituted or unsubstituted heteroaryl.
93. The method of one of embodiments 75 to 89, wherein R¹ is substituted phenyl.
94. The method of one of embodiments 75 to 89, wherein R¹ is phenyl substituted with —CF₃ or halogen.
95. The method of one of embodiments 75 to 94, wherein ring A is substituted or unsubstituted arylene or substituted or unsubstituted heteroarylene.
96. The method of one of embodiments 75 to 94, wherein ring A is substituted or unsubstituted C₆-C₁₀ arylene.
97. The method of one of embodiments 75 to 94, wherein ring A is unsubstituted naphthalenyl.
98. The method of one of embodiments 75 to 97, wherein the Ire1 kinase modulating compound has the formula:

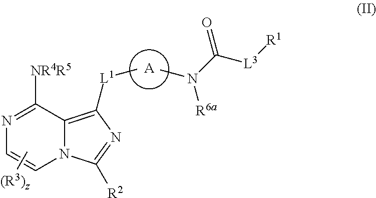

wherein, L³ is a bond, —NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
99. The method of embodiment 98, wherein the Ire1 kinase modulating compound has the formula:

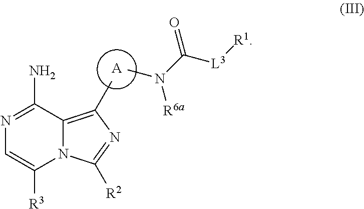

100. The method of embodiment 75, wherein the Ire1 kinase modulating compound is selected from the group consisting of:

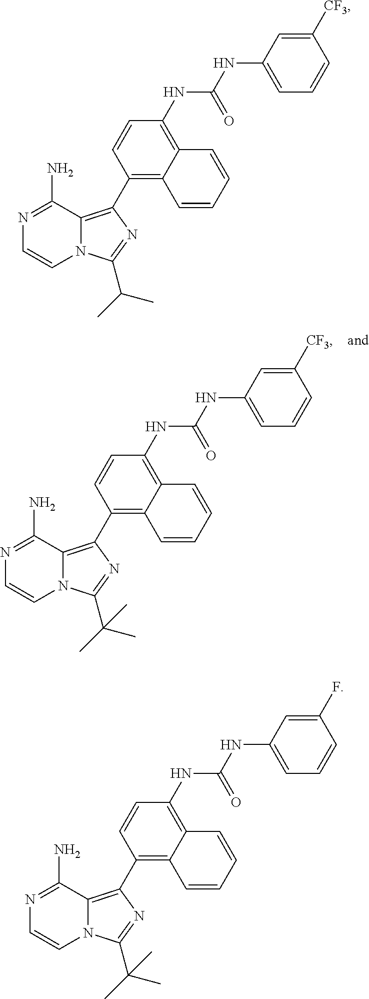 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "and" ; CC(C)(C)c1nc(-c2ccc(NC(=O)Nc3cccc(F)c3)c3ccccc23)c2c(N)nccn12]

101. The method of one of embodiments 75 to 100, wherein said Ire1 kinase modulating compound is administered in a synergistic amount.
102. The method of one of embodiments 75 to 101, wherein said Ire1 ribonuclease modulating compound is a derivative, analog, or prodrug of STF-083010, MKC-3946, 4μ8C, 3-methoxy-6-bromosalicylaldehyde, a salicylaldehyde, or toyocamycin.
103. The method of one of embodiments 75 to 101, wherein said Ire1 ribonuclease modulating compound is STF-083010, MKC-3946, 4μ8C, 3-methoxy-6-bromosalicylaldehyde, a salicylaldehyde, or toyocamycin.
104. The method of one of embodiments 75 to 103, wherein said Ire1 ribonuclease modulating compound is STF-083010.
105. The method of one of embodiments 75 to 104, wherein said Ire1 ribonuclease modulating compound is administered in a synergistic amount.
106. The method of one of embodiments 75 to 105, wherein said Ire1 kinase modulating compound and said Ire1 ribonuclease modulating compound are administered in a combined synergistic amount.
107. A pharmaceutical combination comprising a first amount of an Ire1 kinase modulating compound and a second amount of an Ire1 ribonuclease modulating compound, wherein the first amount and the second amount are together a combined synergistic amount.
108. The pharmaceutical composition of embodiment 107, wherein the Ire1 kinase modulating compound and the Ire1 ribonuclease modulating compound are in a single dosage form.
109. The pharmaceutical composition of embodiment 108, wherein the single dosage form further comprises a pharmaceutically acceptable excipient.
110. The pharmaceutical composition of embodiment 107, wherein the Ire1 kinase modulating compound is in a first dosage form and the Ire1 ribonuclease modulating compound is in a second dosage form.
111. The pharmaceutical composition of embodiment 110, wherein the first dosage form further comprises a first pharmaceutically acceptable excipient and said second dosage form further comprises a second pharmaceutically acceptable excipient.
112. The pharmaceutical composition of embodiment 107, wherein said kinase modulating is kinase inhibiting.
113. The pharmaceutical composition of embodiment 107, wherein said ribonuclease modulating is ribonuclease inhibiting.
114. The pharmaceutical composition of one of embodiments 107 to 113, wherein said Ire1 kinase modulating compound has the formula:

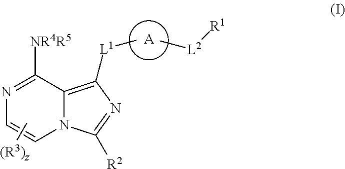

wherein, ring A is substituted or unsubstituted cycloalkylene, substituted or unsubstituted heterocycloalkylene, substituted or unsubstituted arylene, or substituted or unsubstituted heteroarylene; L¹ is a bond or unsubstituted C₁-C₅ alkylene; L² is a bond, —NR^{6a}—, —O—, —S—, —C(O)—, —S(O)—, —S(O)₂—, —NR^{6a}C(O)—, —C(O)NR^{6b}—, —C(O)(CH₂)₂—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R³ is independently hydrogen, oxo, halogen, —CX^{b}₃, —CN, —SO₂Cl, —SO₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁴ and R⁵ are independently hydrogen or unsubstituted C₁-C₆ alkyl; R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl; each occurrence of the symbols n, n1, and n2 is independently an integer from 0 to 4; each occurrence of the symbols m, m1, m2, v, v1, and v2 is independently an integer from 1 to 2; the symbol z is an integer from 0 to 2; the symbol z2 is an integer from 1 to 4; each occurrence of the symbols X, X^{a}, and X^{b} is independently a halogen.
115. The pharmaceutical composition of embodiment 114, wherein R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
116. The pharmaceutical composition of embodiment 114, wherein R³ is hydrogen.
117. The pharmaceutical composition of one of embodiments 114 to 116, wherein the symbol z is 1.
118. The pharmaceutical composition of one of embodiments 114 to 117, wherein R² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
119. The pharmaceutical composition of one of embodiments 114 to 117, wherein R² is substituted or unsubstituted alkyl.
120. The pharmaceutical composition of one of embodiments 114 to 117, wherein R² is substituted or unsubstituted C₁-C₆ alkyl.
121. The pharmaceutical composition of one of embodiments 114 to 117, wherein R² is unsubstituted C₁-C₆ alkyl.
122. The pharmaceutical composition of one of embodiments 114 to 117, wherein R² is unsubstituted isopropyl.
123. The pharmaceutical composition of one of embodiments 114 to 117, wherein R² is unsubstituted tert-butyl.
124. The pharmaceutical composition of one of embodiments 114 to 123, wherein R⁴ and R⁵ are hydrogen.
125. The pharmaceutical composition of one of embodiments 114 to 124, wherein L¹ is a bond.
126. The pharmaceutical composition of one of embodiments 114 to 124, wherein L¹ is unsubstituted methylene.
127. The pharmaceutical composition of one of embodiments 114 to 126, wherein L² is —NR^{6a}C(O)NR^{6b}—.
128. The pharmaceutical composition of embodiment 127, wherein R^{6a} and R^{6b} are hydrogen.
129. The pharmaceutical composition of one of embodiments 114 to 128, wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
130. The pharmaceutical composition of one of embodiments 114 to 128, wherein R¹ is substituted or unsubstituted cycloalkyl, substituted or unsubstituted heterocycloalkyl, substituted or unsubstituted aryl, or substituted or unsubstituted heteroaryl.
131. The pharmaceutical composition of one of embodiments 114 to 128, wherein R¹ is substituted or unsubstituted aryl or substituted or unsubstituted heteroaryl.
132. The pharmaceutical composition of one of embodiments 114 to 128, wherein R¹ is substituted phenyl.
133. The pharmaceutical composition of one of embodiments 114 to 128, wherein R¹ is phenyl substituted with —CF₃ or halogen.
134. The pharmaceutical composition of one of embodiments 114 to 133, wherein ring A is substituted or unsubstituted arylene or substituted or unsubstituted heteroarylene.
135. The pharmaceutical composition of one of embodiments 114 to 133, wherein ring A is substituted or unsubstituted C₆-C₁₀ arylene.
136. The pharmaceutical composition of one of embodiments 114 to 133, wherein ring A is unsubstituted naphthalenyl.
137. The pharmaceutical composition of one of embodiments 114 to 136, wherein the Ire1 kinase modulating compound has the formula:

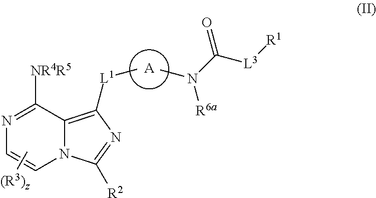

wherein, L³ is a bond, —NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.
138. The pharmaceutical composition of embodiment 137, wherein the Ire1 kinase modulating compound has the formula:

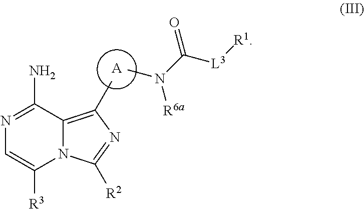

139. The pharmaceutical composition of embodiment 114, wherein the Ire1 kinase modulating compound is selected from the group consisting of:

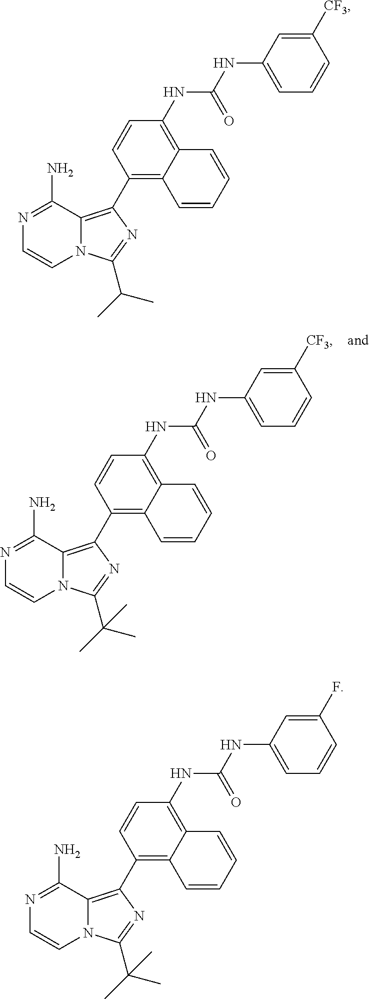 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "and" ; CC(C)(C)c1nc(-c2ccc(NC(=O)Nc3cccc(F)c3)c3ccccc23)c2c(N)nccn12]

140. The pharmaceutical composition of one of embodiments 114 to 139, wherein said Ire1 kinase modulating compound is in a synergistic amount.
141. The pharmaceutical composition of one of embodiments 114 to 140, wherein said Ire1 ribonuclease modulating compound is a derivative, analog, or prodrug of STF-083010, MKC-3946, 4μ8C, 3-methoxy-6-bromosalicylaldehyde, a salicylaldehyde, or toyocamycin.
142. The pharmaceutical composition of one of embodiments 114 to 140, wherein said Ire1 ribonuclease modulating compound is STF-083010, MKC-3946, 4μ8C, 3-methoxy-6-bromosalicylaldehyde, a salicylaldehyde, or toyocamycin.
143. The pharmaceutical composition of one of embodiments 114 to 140, wherein said Ire1 ribonuclease modulating compound is STF-083010.
144. The pharmaceutical composition of one of embodiments 114 to 143, wherein said Ire1 ribonuclease modulating compound is in a synergistic amount.

### G. EXAMPLES

1. Divergent Allosteric Modulation of IRE1α Oligomeric State and RNase Activity with Distinct Kinase Inhibitors.

As with the rationally-engineered mutants, we find that intermediate activation states in IRE1α occur naturally through rare somatic Ire1α gene mutations found in human cancers, including glioblastoma, lung adenocarcinoma and serous ovarian cancer (Greenman et al., 2007). We predicted that five mutations spanning the kinase and RNase should affect function: four are missense, and one, Q780Δ, that is nonsense, amputates the entire RNase. Expressed conditionally in isogenic INS-1 lines, the human IRE1α cancer mutants are all compromised for apoptosis. Normalized to wild-type, the mutations significantly abrogate auto-phosphorylation and XBP1 splicing. Expression of severely crippled IRE1α (Q780Δ) or IRE1α (P830L) actually increases Ins1 mRNA levels, suggesting that some basal decay may even be blocked. Cells expressing IRE1α (Q780Δ) or IRE1α (P830L) proliferate well, in contrast to those expressing IRE1α (WT) or parental lines under ER stress. The mRNA encoding cyclin-dependent kinase inhibitor, p21, is strongly induced in cells expressing IRE1α (WT), but not IRE1α (Q780Δ) or IRE1α (P830L). Marking cycling cells, Ki67 sharply declines upon expression of IRE1α (WT), but not IRE1α (Q780Δ) or IRE1α (P830L).

Lack of the RNase in IRE1α (Q780Δ) converts it into a dominant-negative. The P830L mutation, which occurs at the kinase/RNase junction, may destabilize a dimerization interface (Xue et al., 2011). We predicted and tested that RNase activity in IRE1α (P830L) can be rescued with a kinase inhibitor, as IRE1α (I642G) can with 1NM-PP1. Without 1NM-PP1, IRE1α(I642G) even reduces apoptosis under ER stress, acting as a strong dominant-negative. Another kinase-dead mutant, IRE1α (K599A) (which is also RNase-dead), and an RNase-dead mutant, IRE1α (N906A) are also strongly dominant-negative for apoptosis.

We previously employed two distinct classes of kinase inhibitors—types I and II—to stabilize alternate kinase active site conformations in IRE1α (Wang et al., 2012). APY29 is a type I kinase inhibitor of IRE1α that stabilizes an active kinase domain conformation, which is typically adopted by ATP-bound kinases. By stabilizing the active kinase conformation, type I inhibitors act as ligands that allosterically activate IRE1α's RNase; e.g., 1NM-PP1 is a type I inhibitor of IRE1α (I642G).

Compared to IRE1α* (WT), IRE1α* (P830L) has reduced kinase activity, as the full-length protein does in vivo. In dose-dependent manner, APY29 suppresses residual autophosphorylation by IRE1α* (P830L). IRE1α* (P830L) cannot cleave a FRET-quenched XBP1 RNA mini-substrate (Han et al., 2009), consistent with reduced RNase activity in vivo. But opposite to effects on kinase activity, APY29 increases IRE1α* (P830L)'s oligomeric state to partially rescue RNase activity.

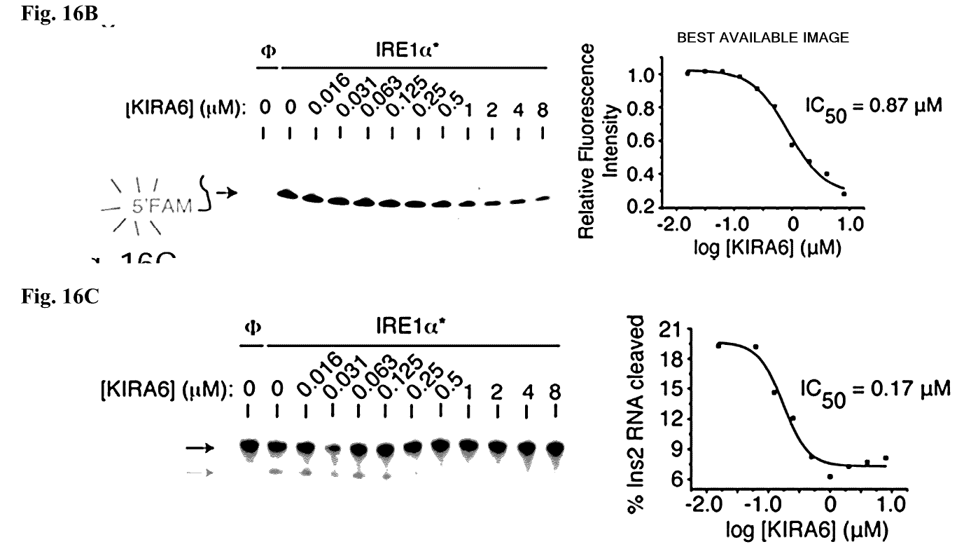

If, as all preceding results suggest, kinase-driven oligomerization of IRE1α hyperactivates its RNase to trigger apoptosis, then kinase inhibitors that block oligomerization should prevent apoptosis under ER stress. To this end, we employed type II kinase inhibitors that stabilize an inactive ATP-binding site conformation in IRE1α. We previously developed a subset of type II kinase inhibitors designated KIRAs, for Kinase-Inhibiting RNase-Attenuators, that inhibit IRE1α's RNase activity by breaking oligomers (Wang et al., 2012). KIRA6 dose-dependently inhibits IRE1α* (WT) kinase activity, XBP1 RNA cleavage, Ins2 RNA cleavage (with lower IC₅₀ than for XBP1 RNA in a competition assay), and oligomerization (FIGS. 16A-16C).

To follow IRE1α oligomerization in vivo, we first tested a reporter called IRE1-3F6HGFP that contains an EGFP domain positioned near the kinase (Li et al., 2010), but found that it has attenuated XBP1 splicing and fails to induce apoptosis. To avoid potential steric effects on the kinase, we constructed a superfolder green fluorescent protein (sfGFP) N-terminally fused to the ER lumenal domain. Expressed isogenically in INS-1 cells, sfGFP-IRE1α retains apoptotic activity and gathers into discrete fluorescent foci in the ER membrane upon exposure to the ER stress agent dithiothreitol (DTT). Also, an (I642G) version of sfGFP-IRE1α fully splices XBP1 mRNA under 1NM-PP1 without forming foci. In fact, without 1NM-PP1, sfGFP-IRE1α (I642G) resists forming foci even under DTT, suggesting that without its ligand it adopts an inactive kinase conformation and explaining dominant-negative effects of IRE1α (I642G). Similar to apoptosis, foci formation by sfGFP-IRE1α (I642G) requires both ER stress and 1NM-PP1, further supporting the tight link between IRE1α oligomerization—shown in vivo through foci—and apoptosis. Thus, using sfGFP-IRE1α, which faithfully recapitulates cytosolic events, we tested and found that KIRA6 prevents foci formation by DTT. Hence, KIRAs fulfill their design principle of breaking kinase/RNase oligomers to inhibit the RNase.

Additional compound synthesis and characterization was conducted as previously described in WO 2014052669 A1, which is incorporated herein by reference in its entirely and for all purposes.

2. Screening and Optimization of IRE1α Modulators

Discovery of ATP-Competitive Kinase Inhibitors that are Able to Inhibit the RNase Domain of IRE1α from a Distance.

Previous studies have demonstrated that the RNase activity of IRE1α is dependent on kinase domain autophosphorylation. Shown herein is an unexpected relationship between the kinase and RNase domains, where ligands that interact with the ATP-binding site of the kinase domain are able to bypass the autophosphorylation requirement and activate the RNase domain (Papa, F. R. et al. Science 302, 1533-1537 (2003)). For instance, the orthogonal ATP-competitive inhibitor 1NM-PP1 is able to rescue the RNase activity of IRE1α mutants that lack kinase activity (Han, D. et al. Biochemical and biophysical research communications 365, 777-783, (2008)). Other ligands that interact with the ATP-binding site of wild-type IRE1α, including the endogenous co-factors ADP and ATP, are also able to activate RNase activity directly (Lee, K. P. et al. Cell 132, 89-100, (2008); Ali, M. M. et al. The EMBO journal 30, 894-905, (2011)). Also, the ATP-competitive inhibitors APY29 and sunitinib directly activate the RNase of yeast and murine IRE1α (Han, D. et al. Cell 138, 562-575, (2009); Korennykh, A. V. et al. Nature 457, 687-693, (2009)). A crystal structure of APY29 bound to yeast IRE1 shows that the kinase catalytic domain is in an active conformation (Korennykh, A. V. et al. Nature 457, 687-693, (2009)), which is a conformation adopted by protein kinases capable of catalyzing phosphate transfer. By stabilizing an active conformation of IRE1α's ATP-binding site, certain co-factors and ATP-competitive inhibitors act as ligands that allosterically activate its adjacent RNase domain. Given the ability to allosterically activate IRE1α's RNase through its kinase domain, it should be possible to also inhibit the RNase through the same kinase domain with a different class of kinase inhibitors that stabilize an inactive ATP-binding site conformation. Two classes of ATP-competitive kinase inhibitors-called types I and II—have been identified, which stabilize alternate kinase active site conformations in numerous protein kinase targets^{51,52}. Type I inhibitors-like APY29 and sunitinib-stabilize an active ATP-binding site conformation. In contrast, type II inhibitors-like the clinically-approved drugs imatinib and sorafenib-selectively stabilize an inactive ATP-binding site conformation⁵³⁻⁵⁵. The inactive ATP-binding site conformation stabilized by type II inhibitors is characterized by outward movement of the catalytically-important Asp-Phe-Gly (DFG) motif, and is therefore called the DFG-out conformation. Described herein is the generation of a diverse panel of type II inhibitors and characterization of their interactions with a number of protein kinases (Krishnamurty, R. et al. Nature chemical biology 9, 43-50, (2013); Han, D. et al. Biochemical and biophysical research communications 365, 777-783, (2008); Brigham, J. L. et al. ACS Chem. Biol. (2013); Hill, Z. B. et al. ACS Chem. Biol. 7, 487-495 (2012)). These pharmacological tools served as a starting point towards the goal of identifying ATP-competitive inhibitors able to allosterically inactivate the IRE1α RNase.

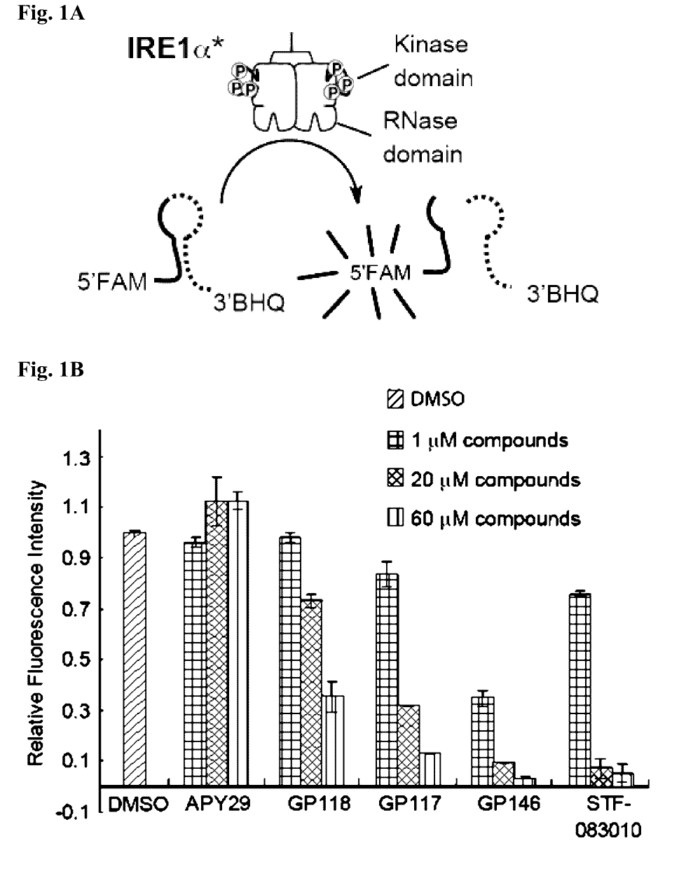

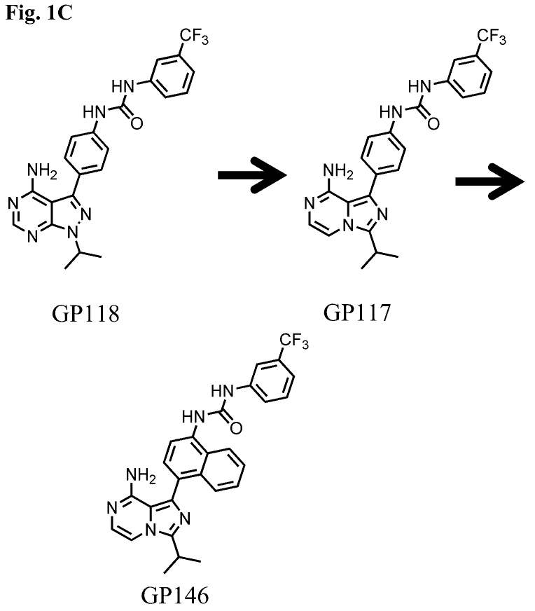

The diverse panel of type II inhibitors were screened for their ability to block the RNase activity of a recombinant soluble human IRE1α mini-protein construct (expressed in baculovirus) containing the kinase/RNase domains—called IRE1α* (Zhang, J. et al. Nature reviews. Cancer 9, 28-39, (2009)). Since IRE1α* is basally autophosphorylated, its RNase is active, and can be assayed using a FRET-quenched XBP1 RNA mini-substrate. While all the type II inhibitors tested with this assay contain the core binding elements predicted to stabilize the DFG-out conformation, only one ligand, demonstrated measurable inhibition of IRE1α*'s RNase activity at a concentration of 60 μM (FIGS. 1B and 1C). Because these type II kinase inhibitors also attenuate the RNase activity of IRE1α*, they were designated KIRAs—for kinase-inhibiting RNase attenuators. KIRA1 is a pyrazolopyrimidine-based inhibitor that has been shown to stabilize the DFG-out conformation of the non-receptor tyrosine kinases Src and Abl (Dar, A. C. et al. Chemistry & Biology 15, 1015-1022 (2008)). Based on the co-crystal structure of KIRA1 bound to Src (PDB: 3EL8) (SEQ ID NO:4) and molecular modeling, proposed contacts with IRE1α are determined

Despite its modest potency, KIRA1 served as a suitable starting point for developing higher affinity allosteric RNase inhibitors. A number of similar analogs were generated and tested for RNase inhibition. While some modifications of KIRA1 were deleterious, replacing the pyrazolopyrimidine scaffold with an imidazopyrazine core provided a significant increase in overall potency (KIRA2, FIGS. 1B and 1C). Furthermore, substituting the 4-anilino group at the C-3 position of KIRA2 with a naphthylamine moiety provided KIRA3. Notably, KIRA3 inhibits XBP1 RNA cleavage to a similar degree as STF-083010, an imine-based small molecule that directly inhibits the IRE1α RNase through covalent modification.

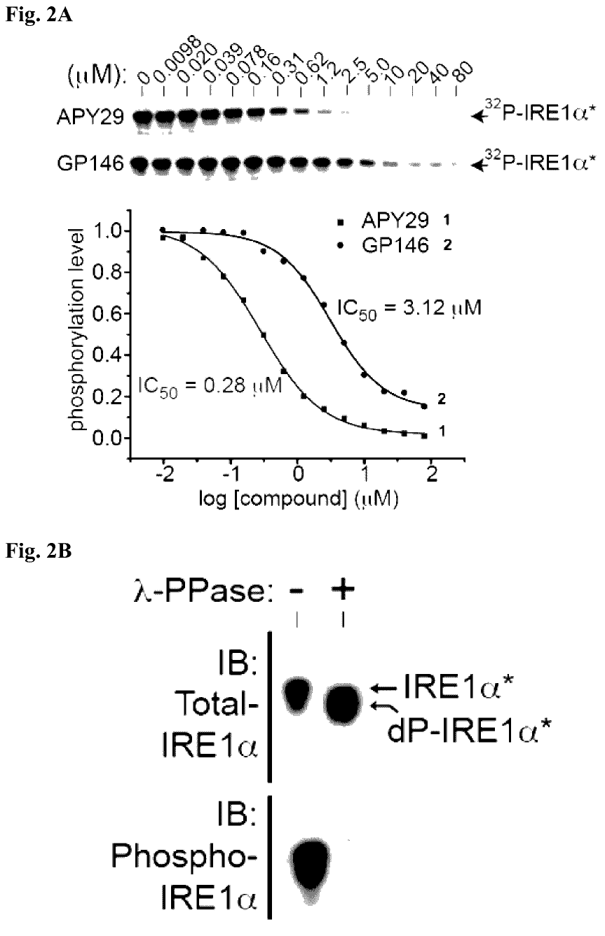

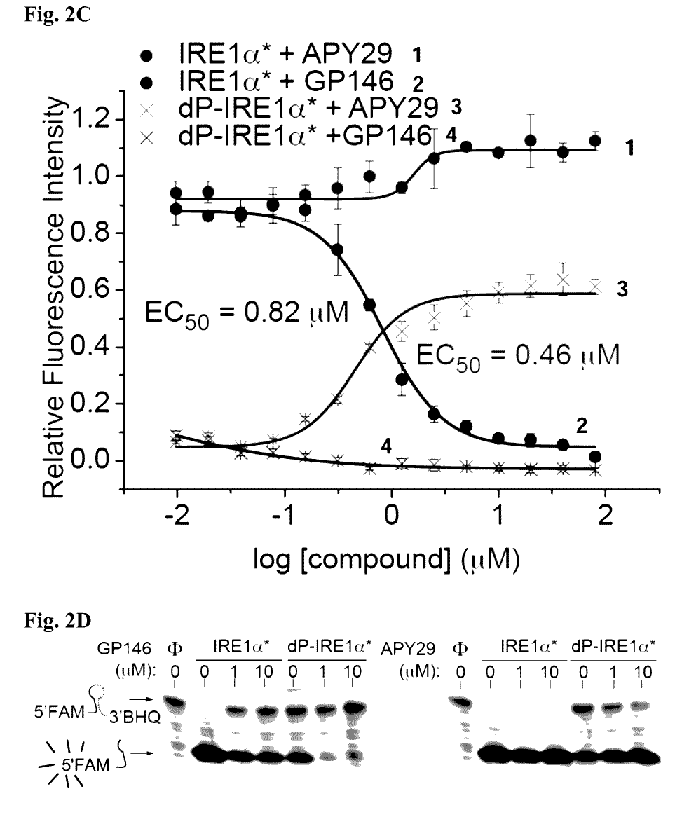

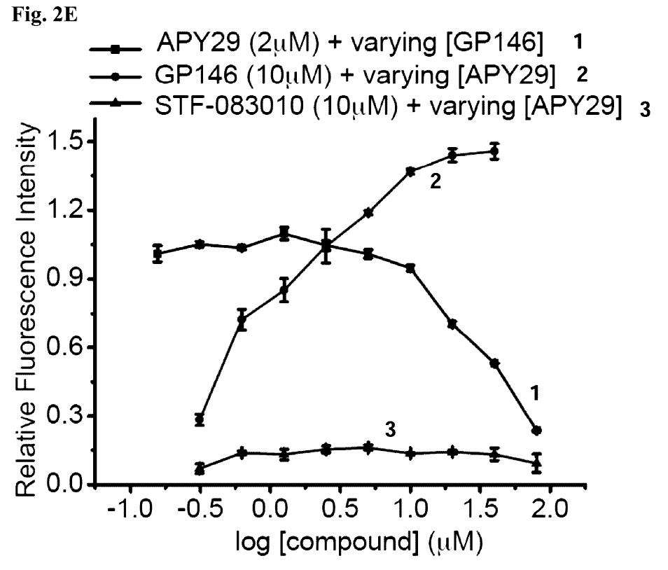

Similar to the type I inhibitor APY29 (IC₅₀ (autophosphorylation)=0.28 μM), KIRA3 (IC₅₀ (autophosphorylation)=3.1 μM) demonstrates dose-dependent reduction of IRE1α* kinase autophosphorylation in vitro. Thus, although KIRA3 and APY29 are both IRE1α* kinase inhibitors, they demonstrate opposing effects on its RNase activity, with APY29 acting as an activator. To further characterize differences between the two kinase inhibitors, a version of IRE1α* was generated with low basal RNase activity by using λ-phosphatase to remove activating phosphates. As expected, the dephosphorylated variant of IRE1α* (dP-IRE1α*) has significantly lower basal RNase activity than IRE1α*; incubating dP-IRE1α* with increasing APY29 progressively restores its ability to cleave the XBP1 mini-substrate, plateauing at ˜60% of the levels of IRE1α* (FIG. 2C). In contrast, KIRA3 suppresses residual RNase activity of dP-IRE1α*. Competition experiments were performed to further explore the opposing effects of APY29 and KIRA3. Increasing concentrations of APY29 progressively reverse IRE1α* RNase inhibition caused by a fixed concentration of KIRA3 (FIG. 2E). Furthermore, the type I inhibitor sunitinib also opposes the RNase inhibitory effect of KIRA3. On the other hand, increasing concentrations of KIRA3 restore RNase inhibition under a fixed concentration of APY29, with an expected increase in the IC₅₀. As predicted, APY29 cannot rescue direct inhibition caused by the covalent RNase modifier STF-083010. Taken together, these results strongly suggest that APY29 and KIRA3 are exerting their opposing effects on RNase activity through the same binding site.

The drug sunitinib is a promiscuous type I inhibitor that has been shown to inhibit the kinase activity of yeast and human IRE1α16,19. To investigate the differences between GP146 (KIRA3) and other ATP-competitive inhibitors of IRE1α, the interaction of sunitinib with the IRE1α* and dP-IRE1α* constructs was further characterized. As expected, sunitinib is a dose-dependent inhibitor of the autophosphorylation activity of IRE1α* (FIG. 11A). In addition, sunitinib activates the RNase activity of dP-IRE1α*, which is consistent with its type I pharmacophore (FIG. 11B). Therefore, both APY29 and sunitinib stabilize an ATP-binding site conformation that activates the RNase domain of IRE1α. Like APY29, increasing amounts of sunitinib are able to rescue the RNase activity of IRE1α* in the presence of a fixed concentration of GP146 (FIG. 11C). Together, these results show that GP146 opposes the stereotypic RNase activation demonstrated by various type I ATP-competitive inhibitors of IRE1α.

KIRA3 Prevents Dimerization and Oligomerization of IRE1α.

Self-association of kinase/RNase monomers has been reported to increase RNase activity as dimers and/or higher-order oligomers form in yeast and mammalian IRE1 proteins. furthermore, the degree of order may correlate directly with activity. Thus, APY29 and KIRA3 were used to test the prediction that they would divergently affect the oligomerization state of human IRE1α as a basis for their opposing effects on its RNase activity. Specifically, RNase activators should drive monomers into higher-order species from baseline. To test this, increasing concentrations of IRE1α* were incubated with either DMSO, or saturating concentrations of APY29 or KIRA3 and the ratio of oligomeric—defined as all species greater than monomers—to monomeric IRE1α was determined. In the absence of ligands, IRE1α* shows a concentration-dependent increase in the oligomer/monomer ratio. APY29 further enhances—whereas KIRA3 decreases—this concentration-dependent increase in the IRE1α* oligomer/monomer ratio. Taken together, the in vitro data support a model in which these two classes of kinase inhibitors divergently modulate IRE1α* RNase activity by exerting opposing effects on the oligomerization state of the enzyme.

3. Synthesis and Characterization of Compositions

Unless otherwise noted, all reagents were obtained from commercial suppliers and used without purification. TLC was performed on EMD Millipore silica gel 60 F254 plates. 1H-NMR spectra were obtained on a Bruker AV-300 or AV301 instrument at room temperature and 13C-NMR spectra were obtained on a Bruker AV-500 instrument at room temperature. Chemical shifts are reported in ppm, and coupling constants are reported in Hz. 1H and 13C resonances are referenced to residual MeOH. Mass spectrometry was performed on a Bruker Esquire Ion Trap MS instrument. The purity of all final compounds was determined by analytical HPLC with two different solvent systems. Analytical conditions A: [C18 (150×2.1 mm), CH3CN/H2O-0.1% CF3CO2H=1:99 to 100:0 over 33 min; 1 mL/min; 220 and 254 nm detection for 33 min]. Analytical conditions B: [C18 (150×2.1 mm), CH3OH/H2O-0.1% CF3CO2H=1:99 to 100:0 over 33 min; 1 mL/min; 220 and 254 nm detection for 33 min].

STF-083010, Sunitinib, nilotinib, and GP21 were obtained from commercial suppliers. All compounds were verified to be >95% pure by analytical HPLC. APY29 and GP118 were prepared according to a previously reported procedures.

1-iodo-3-isopropylimidazo[1,5-a]pyrazin-8-amine (compound 1). Compound 1 was synthesized according to a previously described procedure. 1-(4-(4,4,5,5-tetramethyl-1,3,2-dioxaborolan-2-yl)phenyl)-3-(3-(trifluoromethyl)phenyl)urea (compound 2). A mixture of 4-Aminophenylboronic acid pinacol ester (50.0 mg, 0.23 mmol) and 3-(Trifluoromethyl)phenyl isocyanate (46.0 uL, 0.31 mmol) in THF (1.9 mL) was stirred overnight at room temperature. The mixture was concentrated, diluted with dichloromethane and washed with water. The organic layer was dried over Na2SO4, concentrated in vacuo and the resultant crude product was purified by flash chromatography (20% ethyl acetate in hexanes) to afford 92.5 mg of compound 2 (98% yield). TLC (hexanes:EtOAc, 80:20 v/v): Rf=0.4; 1H NMR (300 MHz, MeOD): δ 7.90 (s, 1H), 7.71-7.68 (m, 2H), 7.60 (d, J=6.0 Hz, 1H), 7.48-7.42 (m, 3H), 7.29 (d, J=9.0 Hz, 1H), 1.34 (s, 12H); ESI-MS (m/z): [M]+ calcd. for C20H22BF3N203, 406.17; [M+1]+ found, 407.5. 1-(4-(4,4,5,5-tetramethyl-1,3,2-dioxaborolan-2-yl)naphthalen-1-yl)-3-(3-(trifluoromethyl)phenyl)urea (compound 3). A mixture of 4-Aminonaphthalene-1-boronic acid pinacol ester (31.2 mg, 0.11 mmol) and 3-(Trifluoromethyl)phenyl isocyanate (21.0 μL, 0.31 mmol) in THF (0.9 mL) was stirred over night at room temperature. The mixture was concentrated, diluted with dichloromethane and washed with water. The organic layer was dried over Na2SO4, concentrated in vacuo and the resultant crude product was purified by flash chromatography (20% ethyl acetate in hexanes) to afford 43.6 mg of compound 3 (86% yield). TLC (hexanes:EtOAc, 80:20 v/v): Rf=0.4; 1H NMR (300 MHz, MeOD): δ 8.87-8.82 (m, 1H), 8.11-7.91 (m, 4H), 7.67 (d, J=9.0 Hz, 1H), 7.60-7.47 (m, 3H), 7.36-7.31 (m, 1H), 1.44 (s, 12H); ESI-MS (m/z): [M]+ calcd. for C24H24BF3N2O3, 456.18; [M+1]+ found, 457.3. N-(4-(4,4,5,5-tetramethyl-1,3,2-dioxaborolan-2-yl)naphthalen-1-yl)-3-(trifluoromethyl)benzamide (compound 4). 4-Aminonaphthalene-1-boronic acid pinacol ester (75.0 mg, 0.267 mmol), 3-(Trifluoromethyl)benzoic acid (67.5 mg, 0.348 mmol), HOBt (55.1 mg, 0.348 mmol), EDCI (68.0 mg, 0.348 mmol) and DIPEA (141 μL, 0.803 mmol) were dissolved in DMF (790 L) and stirred overnight at room temperature. The crude mixture was diluted in ethyl acetate and washed with NH4Cl and Na2CO3. The organic layer was dried over Na2SO4 and concentrated in vacuo. The crude organic product was purified by column chromatography (20% ethyl acetate in hexanes) to afford 16.6 mg of compound 4 (14% yield). TLC (hexanes:EtOAc, 80:20 v/v): Rf=0.4; 1H NMR (300 MHz, MeOD) δ 8.87-8.84 (m, 1H), 8.41-8.36 (m, 2H), 8.13-8.04 (m, 2H), 8.01-7.96 (m, 1H), 7.85-7.78 (m, 1H), 7.68 (d, J=6.0 Hz, 1H), 7.58-7.55 (m, 2H), 1.47 (s, 12H); ESI-MS (m/z): [M]+ calcd. for C24H23BF3NO3, 441.17; [M+1]+ found, 442.2. 8-chloro-1-iodo-3-isopropylimidazo[1,5-a]pyrazine (compound 5). Compound 5 was synthesized according to previously published procedure. 1-iodo-3-isopropyl-N-methylimidazo[1,5-a]pyrazin-8-amine (compound 6). Compound 5 (21.8 mg, 0.068 mmol) was dissolved in a solution of 40% MeNH2 in MeOH (91 L) and was stirred at 80° C. for 2 h in a microwave. The reaction mixture was concentrated in vacuo to obtain 20.3 mg of compound 6 (94% yield). TLC (hexanes:EtOAc, 50:50 v/v): Rf=0.2; 1H NMR 1H NMR (300 MHz, MeOD) δ 7.51 (d, J=6.0 Hz, 1H), 7.04 (d, J=6.0 Hz, 1H), 3.42-3.34 (m, 1H), 3.07 (s, 3H), 1.37 (d, J=6.0 Hz, 6H); ESI-MS (m/z): [M]+ calcd. for C10H13IN4, 316.02; [M+1]+ found, 317.1.

1-(4-(8-amino-3-isopropylimidazo[1,5-a]pyrazin-1-yl)phenyl)-3-(3-(trifluoromethyl)phenyl)urea (KIRA2). A mixture of compound 1 (22.1 mg, 0.073 mmol), compound 2 (35.5 mg, 0.087 mmol), tetrakis(triphenylphosphine)palladium (2.6 mg, 2.1 μmol) and sodium carbonate (17.0 mg, 0.161 mmol) was dissolved in a 3:1 mixture of DME/water (280 uL). The mixture was heated overnight at 85° C. The crude mixture was then allowed to cooled to room temperature, diluted in a mixture of acetonitrile/water and purified by reverse phase chromatography (HPLC) to obtain 11.5 mg of KIRA2 (35% yield). TLC (CH2Cl2:MeOH, 95:5 v/v): Rf=0.5; 1H NMR (300 MHz, MeOD): δ 7.95 (s, 1H), 7.68-7.64 (m, 1H), 7.65-7.63 (m, 2H), 7.60-7.56 (m, 2H), 7.55-7.48 (m, 2H), 7.34-7.31 (m, 1H), 7.02-7.00 (dd, J=6.0 Hz, J=3.0 Hz, 1H), 3.49-3.43 (m, 1H), 1.44 (d, J=6.0 Hz, 6H); ESI-MS (m/z): [M]+ calcd. for C23H21F3N6O, 454.17; [M+1]+ found, 455.5. HPLC Purification Conditions: C18 column (250×21 mm), CH3CN/H2O-0.1% CF3CO2H=1:99 to 100:0 over 78 min; 8 mL/min; 220 nm and 254 nm detection for 78 min. The purity of GP 117 was determined to be >98% by analytical HPLC in two different solvent systems.

1-(4-(8-amino-3-isopropylimidazo[1,5-a]pyrazin-1-yl)naphthalen-1-yl)-3-(3-(trifluoromethyl)phenyl)urea (KIRA3). A mixture of compound 1 (12.0 mg, 0.040 mmol), compound 3 (21.9 mg, 0.048 mmol), tetrakis(triphenylphosphine)palladium (1.4 mg, 1.2 μmol) and sodium carbonate (9.3 mg, 0.088 mmol) was dissolved in a 3:1 mixture of DME/water (160 uL). The mixture was heated overnight at 85° C. The crude mixture was cooled to room temperature, diluted in a mixture of acetonitrile/water and purified by reverse phase chromatography (HPLC) to obtain 12.3 mg of GP146 (61% yield). TLC (CH2Cl2:MeOH, 95:5 v/v): Rf=0.4; 1H NMR (300 MHz, MeOD): δ 8.27-8.22 (m, 1H), 8.02-7.96 (m, 2H), 7.90-7.86 (m, 1H), 7.83-7.79 (m, 1H), 7.72-7.49 (m, 5H), 7.37-7.32 (m, 1H), 7.04-6.99 (m, 1H), 3.66-3.55 (m, 1H), 1.54-1.48 (m, 6H); 13C NMR (500 MHz, MeOD): δ 154.9, 151.6, 149.8, 140.2, 135.9, 132.9, 129.4, 128.7, 128.7, 127.1, 126.6, 126.6, 125.8, 125.7, 121.9, 121.9, 121.8, 120.2, 118.7, 118.7, 115.1, 114.6, 112.9, 108.4, 25.8, 19.6; [M+1]+ found, 505.4. HPLC Purification Conditions: C18 column (250×21 mm), CH3CN/H2O-0.1% CF3CO2H=1:99 to 100:0 over 78 min; 8 mL/min; 220 nm and 254 nm detection for 78 min. The purity of GP146 was determined to be >98% by analytical HPLC in two different solvent systems.

1-(4-(3-isopropyl-8-(methylamino)imidazo[1,5-a]pyrazin-1-yl)naphthalen-1-yl)-3-(3-(trifluoromethyl)phenyl)urea (GP146(NMe)). A mixture of compound 6 (11.1 mg, 0.035 mmol), compound 3 (19.3 mg, 0.042 mmol), Tetrakis(triphenylphosphine)palladium (1.3 mg, 1.0 umol) and sodium carbonate (8.2 mg, 0.08 mmol) was dissolved in a 3:1 mixture of DME/water (130 uL). The mixture was heated overnight at 85° C. The crude mixture was cooled to room temperature, diluted in a mixture of acetonitrile/water and purified by reverse phase chromatography (HPLC) to obtain 5.0 mg of the GP146(NMe) (27% yield). TLC (CH2Cl2:MeOH, 95:5 v/v): Rf=0.6; 1H NMR (300 MHz, MeOD) δ 8.26 (d, J=9.0 Hz, 1H), 8.03 (s, 1H), 7.96 (d, J=9.0, 1H), 7.90 (d, J=6.0 Hz, 1H), 7.84 (d, J=9.0 Hz, 1H), 7.72-7.66 (m, 3H), 7.62-7.51 (m, 2H), 7.37-7.34 (m, 1H), 7.04 (d, J=6.0 Hz, 1H), 3.66-3.57 (m, 1H), 2.99 (s, 3H), 1.50 (dd, J=15.0, 6.0 Hz, 6H); 13C NMR (500 MHz, MeOD): δ 154.99, 151.22, 148.86, 140.19, 135.97, 133.04, 131.68, 129.36, 128.96, 128.80, 128.64, 128.54, 126.97, 126.63, 125.86, 125.80, 121.97, 121.84, 120.45, 118.68, 114.99, 114.55, 113.05, 108.47, 28.25, 25.75, 19.60; ESI-MS (m/z): [M]+ calcd. for C28H25F3N60, 518.2; [M+1]+ found, 519.5. The purity of GP146(NMe) was determined to be >98% by analytical HPLC.

N-(4-(8-amino-3-isopropylimidazo[1,5-a]pyrazin-1-yl)naphthalen-1-yl)-3-(trifluoromethyl)benzamide (GP146(Am)). A mixture of compound 1 (9.5 mg, 0.031 mmol), compound 4 (16.6 mg, 0.038 mmol), Tetrakis(triphenylphosphine)palladium (1.1 mg, 0.94 μmol) and sodium carbonate (7.3 mg, 0.069 mmol) was dissolved in a 3:1 mixture of DME/water (120 L). The mixture was heated overnight at 85° C. The crude mixture was cooled to room temperature, diluted in a mixture of acetonitrile/water and purified by reverse phase chromatography (HPLC) to obtain 5.3 mg of GP146(Am) (34% yield). TLC (CH2Cl2:MeOH, 95:5 v/v): Rf=0.5; 1H NMR (300 MHz, MeOD) δ 8.46 (s, 1H), 8.42 (d, J=6.0 Hz, 2H), 8.16-8.13 (m, 1H), 8.01-7.98 (m, 1H), 7.86-7.84 (m, 1H), 7.81-7.77 (m, 2H), 7.72-7.69 (m, 1H), 7.68-7.62 (m, 2H), 7.60-7.54 (m, 1H), 7.06 (d, J=6.0 Hz, 1H), 3.61-3.52 (m, 1H), 1.53-1.46 (m, 6H); ESI-MS (m/z): [M]+ calcd. for C27H22F3N50, 489.18; [M+1]+ found, 490.4 The purity of GP146(Am) was determined to be >98% by analytical HPLC.

1-(4-(8-amino-3-tert-butylimidazo[1,5-a]pyrazin-1-yl)naphthalen-1-yl)-3-(3-(trifluoromethyl)phenyl)urea (KIRA6). A mixture of 1-iodo-3-tertbutylimidazo[1,5-a]pyrazin-8-amine (60.0 mg, 0.120 mmol), 3 (66 mg, 0.15 mmol), tetrakis(triphenylphosphine)palladium (5 mg, 4 μmol) and sodium carbonate (928 mg, 0.27 mmol) was dissolved in a 3:1 mixture of DME/water (0.5 mL). The mixture was heated overnight at 85° C. The crude mixture was cooled to room temperature, diluted in a mixture of acetonitrile/water and purified by reverse phase chromatography (HPLC) to obtain 53 mg of KIRA6. TLC (CH2Cl2:MeOH, 95:5 v/v): Rf=0.4; 1H NMR (300 MHz, MeOD): δ 8.26 (m, 1H), 8.08-7.99 (m, 2H), 7.90-7.86 (m, 1H), 7.83-7.79 (m, 1H), 7.69-7.52 (m, 5H), 7.35 (d, J=7.4 Hz, 1H), 6.98 (m, 1H), 1.65 (s, 9H); 13C NMR (126 MHz, MeOD): δ 154.8, 140.2, 135.7, 133.0, 132.4, 131.7, 131.6, 131.0, 130.7, 129.4, 128.8, 128.6, 128.5, 127.0, 126.6, 125.9, 125.4, 123.2, 121.9, 120.1, 118.7, 115.0, 114.4, 110.1, 33.6, 27.3; ESI-MS (m/z): [M]+ calcd. for C28H25F3N60 (M+H+): 519.2; found 519.4.

Generate Potent and Selective Reversible Type II Inhibitors of IRE1α.

As described above, a type II ATP-competitive inhibitor (KIRA3) that can inactivate IRE1α RNase and kinase activities has been identified, in vitro and in vivo (Zhang, J. et al. Nature reviews. Cancer 9, 28-39, (2009)). Described herein are efforts to increase the potency and selectivity of these type II inhibitors. Using structure-based drug design, KIRA3 was further optimized. Initially, a homology model of the IRE1α ATP-binding site in the DFG-out conformation, will be used to guide analog synthesis, including use of structures of KIRAs bound to on-targets (IRE1α) and off-targets (Src) to refine the inhibitor docking protocol. Described herein is the development of irreversible inhibitors that target a non-conserved cysteine in IRE1α's activation loop. Described herein is the development of KIRA3 analogs that contain an electrophile that targets a cysteine predicted to be accessible when IRE1α is in the DFG-out conformation. Details are described below.

KIRA Analog Synthesis:

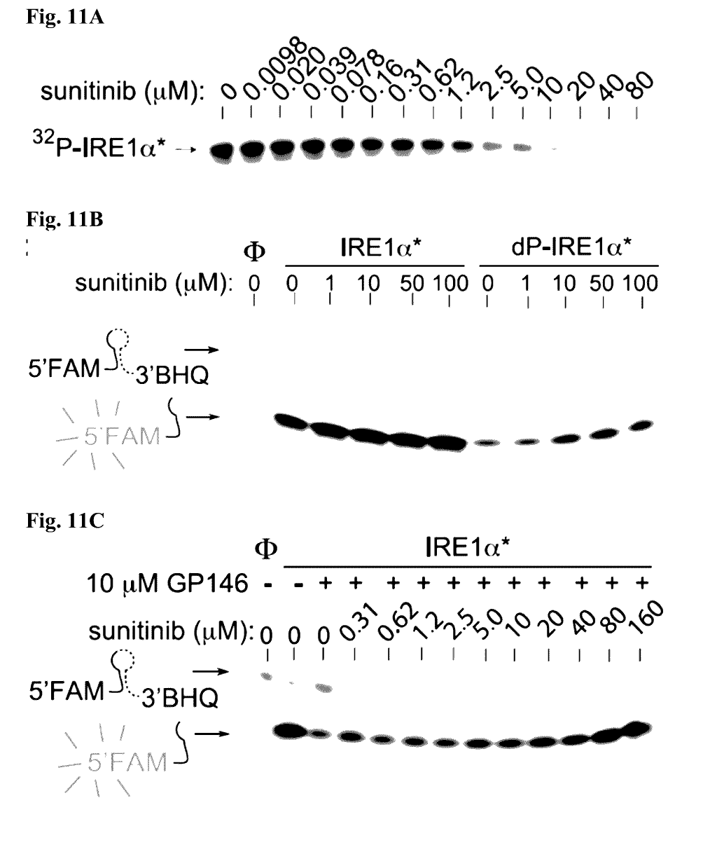

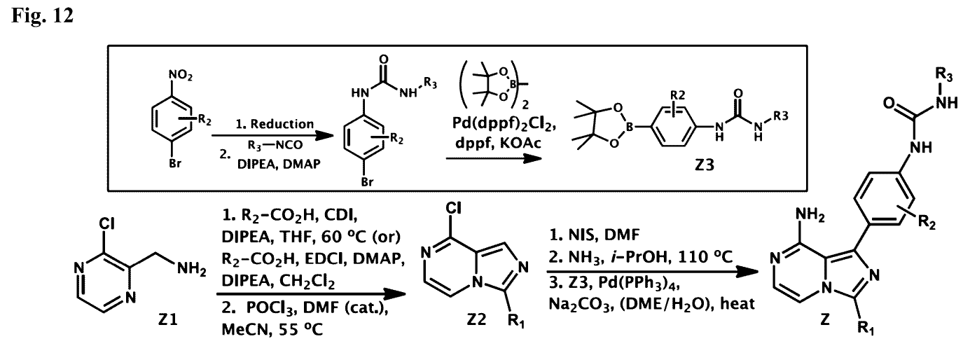

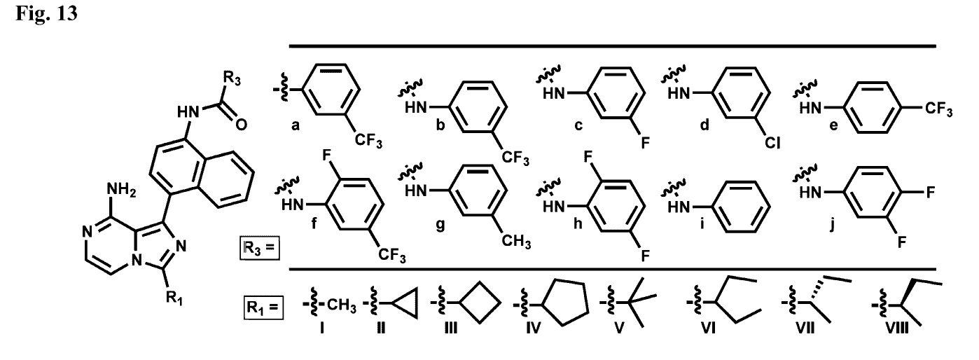

The synthetic strategy for generating inhibitors of general structure Z is shown FIG. 12. Acylation of commercially available amine Z1 with carboxylic acids (R₁—CO₂H) that have been activated with 1,1′-carbonyldiimidazole (CDI) (or activated with EDCI, DMAP), followed by cyclization with POCl₃ generates imidazopyrimidines (Z2) that are substituted at the 1-position (R₁) (Mulvihill, M. J. et al. Bioorg. Med. Chem. 16, 1359-1375 (2008); Mulvihill, M. J. et al. Bioorganic & Medicinal Chemistry Letters 17, 1091-1097 (2007)). Urea substituents are introduced at the C-3 position by iodinating the scaffold with NIS, nucleophilic substitution with ammonia in isoproponal (in a sealed reaction vessel) and palladium-mediated Suzuki couplings to urea-containing boronic esters (prepared as shown in the box) (Jin, M. et al. Bioorganic & Medicinal Chemistry Letters 21, 1176-1180 (2011); Wang, J.-X. et al. Org. Lett. 10, 2923-2926 (2008); Board, J. et al. Org. Lett. 11, 5118-5121 (2009)). An alternate synthetic route that introduces substituents at the C-3 position (R₂ substituents) without using a metal-mediated cross-coupling can also be used (Mulvihill, M. J. et al. Bioorganic & Medicinal Chemistry Letters 17, 1091-1097 (2007)). Over 40 analogs have been generated using this synthetic methodology. Representative inhibitors are shown in FIG. 13. KIRA6 (R₁=V; R₃=B), a more potent (IC₅₀(RNase)=210 nM; IC₅₀(kinase)=620 nM) analog of KIRA3, is extensively profiled in Aim 2. KIRA7 (R₁=V; R₃=C) is one of the most potent KIRA (IC₅₀(RNase)=<30 nM; IC₅₀(kinase)=35 nM) generated to date.

Irreversible KIRAs that Target Cys715 in the Activation Loop.

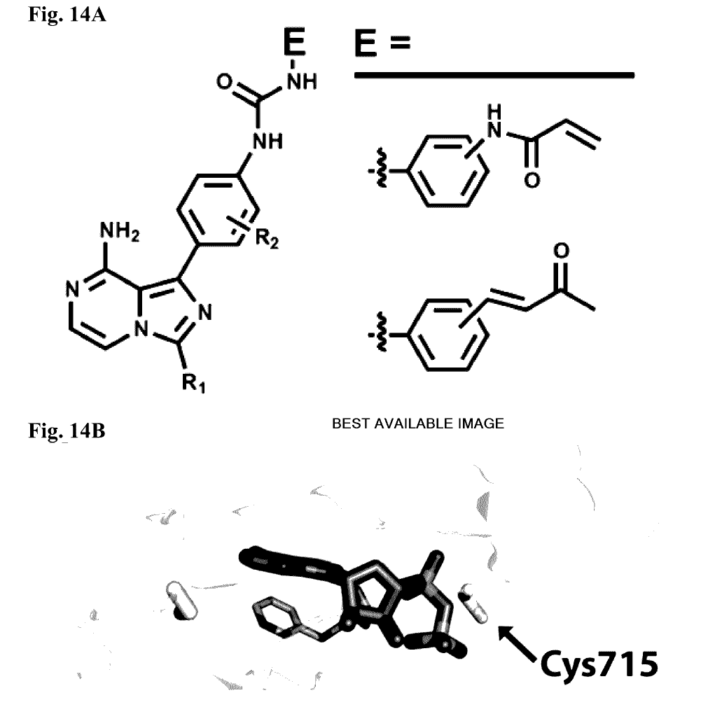

The IRE1α kinase domain possesses a cysteine residue (Cys715) two residues C-terminal to the DFG motif (FIGS. 14A-14B). Cys715 is rapidly alkylated with haloacetamide-containing ICAT reagents, and the rate is increased under KIRA3 (Zhang, J. et al. Nature reviews. Cancer 9, 28-39, (2009)). Modeling suggests that Cys715 is in close proximity to the R₃ substituent of the KIRA scaffold when the DFG motif is in the “out” conformation (the conformation the IRE1α ATP-binding site adopts when bound to KIRAs). Of the 518 kinases in the human kinome, 42 have a cysteine residue at an equivalent or adjacent position (Leproult, E. et al. J. Med. Chem. 54, 1347-1355 (2011)). However, chemical proteomic profiling studies with type II inhibitors suggest that none of these 42 kinases, except IRE1α, are able to adopt the DFG-out inactive conformation (Krishnamurty, R. et al. Nature chemical biology 9, 43-50, (2013); Brigham, J. L. et al. ACS Chem. Biol. 130123155823009). Therefore, it should be possible to selectively target Cys715 with a properly oriented electrophile displayed from the KIRA scaffold. Representative irreversible KIRAs that are being generated are shown in FIGS. 14A-14B (electrophiles will be introduced in the last synthetic step to avoid instability issues during the palladium-mediated cross coupling). A number of highly selective irreversible kinase inhibitors have been developed by targeting non-conserved cysteine residues (Kwarcinski, F. E. et al. ACS Chem. Biol. 7, 1910-1917 (2012); Barouch-Bentov, R. et al. Molecular Cell 33, 43-52 (2009); Zhang, T. et al. Chemistry & Biology 19, 140-154 (2012); Zhou, W. et al. Bioorganic & Medicinal Chemistry Letters 21, 638-643 (2011); Zhou, W. et al. Chemistry & Biology 17, 285-295 (2010); Zhou, W. et al. Nature 462, 1070-1074 (2009); Serafimova, I. M. et al. Nature Chemical Biology 8, 471-476 (2012); Henise, J. C. & Taunton, J. J. Med. Chem. 54, 4133-4146 (2011); Cohen, M. S. f et al. Science 308, 1318-1321 (2005)).

4. Analysis of the GP146-IRE1α and APY29-IRE1α Interactions.

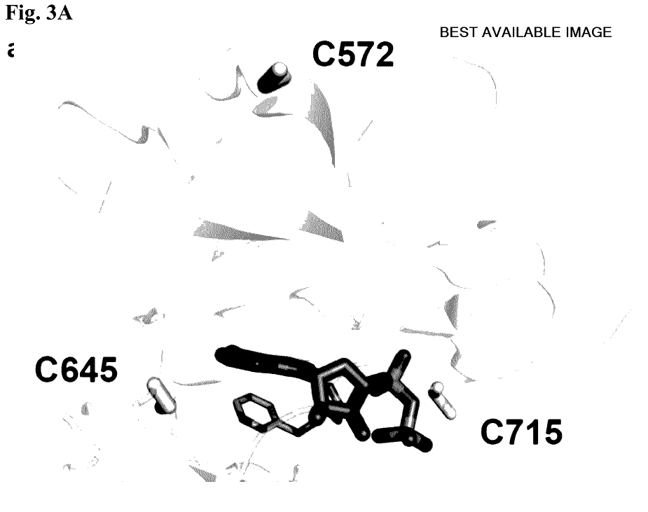

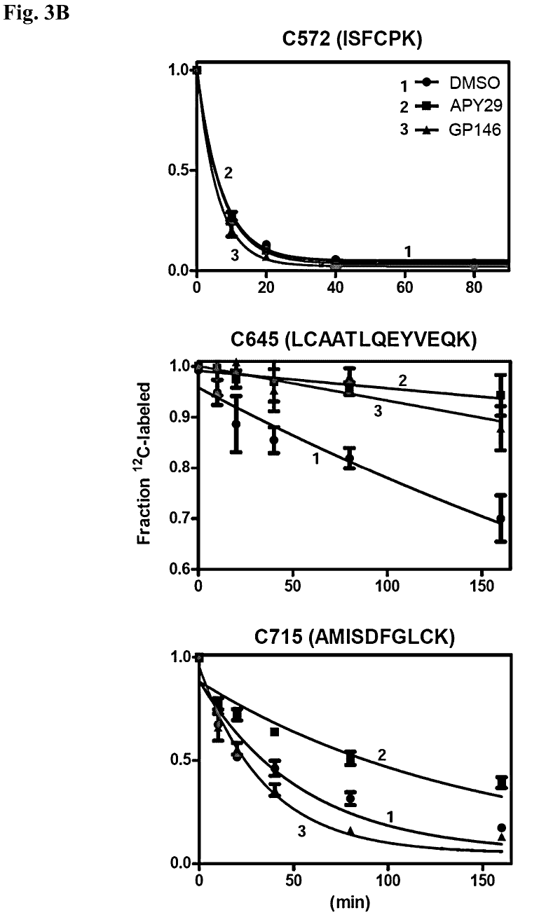

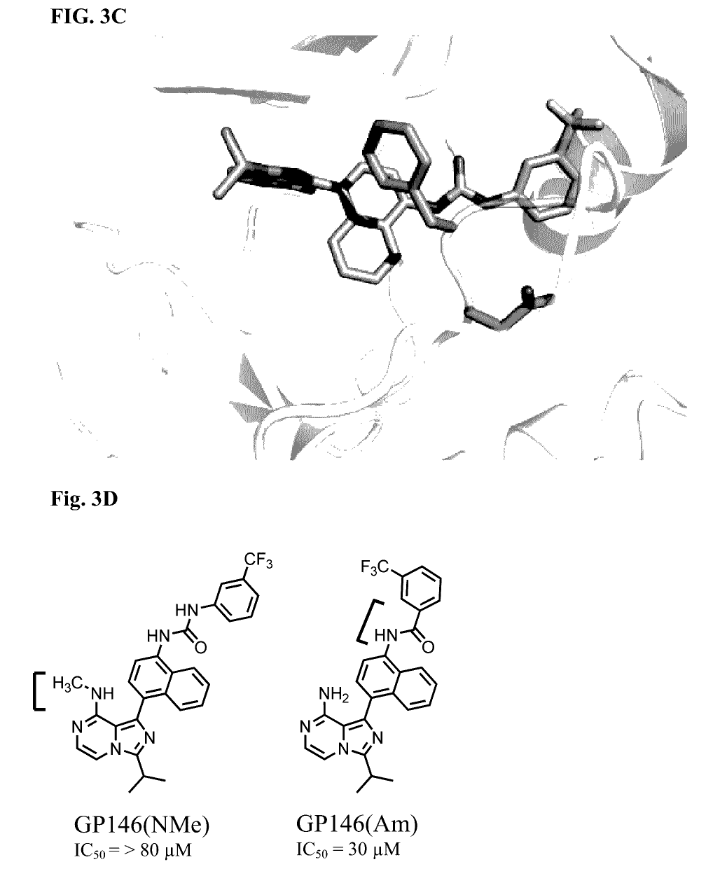

To further confirm that AYP29 and GP146 are exerting their opposing effects through the same ATP-binding site, a series of biochemical footprinting experiments were conducted (Tu, B. P. & Wang, J. C. Proc. Natl. Acad. Sci. USA 96, 4862-4867 (1999); Underbakke, E. S. et al. Angew. Chem. Int. Ed. 47, 9677-9680 (2008)). Specifically, the accessibility of three native cysteine residues within human IRE1α (Cys572, Cys645, and Cys715) to alkylating agents in the presence or absence of APY29 and GP146 was determined (FIG. 3A). For these studies, electrophilic isotope-coded affinity tag (ICAT) reagents were used to allow a ratiometric and, therefore, quantitative comparison of alkylation rates (Underbakke, E. S. et al. Angew. Chem. Int. Ed. 47, 9677-9680 (2008)). As Cys645 and Cys715 are located within the ATP-binding cleft of IRE1α, the accessibility of these residues would be expected to be affected by ligands that occupy this site, while Cys572 is a solvent-exposed residue located on the top of the N-terminal lobe of the kinase. Consistent with both APY29 and GP146 occupying the ATP-binding site of IRE1α, Cys645, which is located in the kinase hinge region, is highly shielded from alkylating agents in the presence of either inhibitor (FIG. 3B). In contrast, these inhibitors exert opposing effects on the accessibility of Cys715, with APY29 slowing the rate of alkylation and GP 146 increasing it. Cys715 is located in the activation loop of IRE1α (two residues C-terminal to the DFG-motif) and the divergent influence of APY29 and GP146 on this residue is concordant with these ligands stabilizing different conformations of the activation loop (FIG. 3B). As expected, no detectable difference in the accessibility of Cys572, which is distal to the kinase active site of IRE1α, is observed in the presence of either inhibitor.

Next, molecular docking experiments were performed to obtain a better understanding of how GP146 and APY29 interact with the ATP-binding site of human IRE1α. A model of the DFG-in ATP-binding site conformation was generated from a co-crystal structure of human IRE1α bound to ADP (PDB code 3P23, chain A) (SEQ ID NO:5) (Ali, M. M. et al. EMBO J. 30, 894-905 (2011)). As a structure of IRE1α in the DFG-out conformation has not yet been described, a homology model of this form was generated by using the activation loop of another kinase, the tyrosine kinase Abl2, as a template. Both the DFG-in and DFG-out models were optimized using multi-step all-atom minimization and explicit water molecular dynamics (MD) simulations (Bowers, K. J. et al. in Proceedings of the ACM/IEEE Conference on Supercomputing (SC06) (Tampa, Fla., USA, 2006)). Predictably, the docked structure of APY29 bound to the DFG-in conformation of human IRE1α is similar to that of this ligand bound to the yeast IRE1 enzyme (Korennykh, A. V. et al. Nature 457, 687-693 (2009)). The pyrazole ring of APY29 forms hydrogen bonds with the kinase hinge region and the pyrimidine moiety occupies the adenine pocket. Attempts to obtain a favorable pose of APY29 bound to the DFG-out conformation of IRE1α were unsuccessful, which is consistent with the ability of this ligand to exclusively stabilize the active conformation of the ATP-binding site.

The most favorable docking pose for GP146 bound to the DFG-out conformation of IRE1α is shown in FIG. 3C. In this pose, the pyrazolopyrimidine ring of this ligand forms two hydrogen bonds with the hinge region and occupies the adenine pocket. The bulky naphthyl ring of GP146 adopts an almost orthogonal conformation relative to the core scaffold and stacks against the Ile gatekeeper residue. Like other type II inhibitors, the trifluoromethylphenyl moiety of GP146 occupies the hydrophobic pocket created by movement of the Phe sidechain in the DFG-motif. While GP146 is well accommodated in the DFG-out conformation of human IRE1α, no favorable poses were observed for this inhibitor bound to the DFG-in conformation. Indeed, the docking studies predict that the only way that GP146 can bind to IRE1α without movement of the DFG motif in the activation loop is if the inhibitor disrupts canonical interactions with the hinge region of the kinase.

To further experimentally test the docking model, analogs of GP146 that contain structural elements predicted to lower inhibitor potency were generated (FIG. 3D). GP146(NMe) contains an N-methyl group that would be predicted to disrupt its interaction with the hinge region of IRE1α, and the amide linkage of GP146(Am) should not allow the trifluoromethylphenyl moiety to form as favorable interactions with the hydrophobic pocket created by movement of the DFG-motif. Consistent with the model, both GP146(NMe) and GP(146Am) show a markedly diminished ability to inhibit the RNase activity of IRE1α compared to GP146 (FIG. 3D).

5. GP146 and APY29 Divergently Affect the Oligomerization State of Ire1α

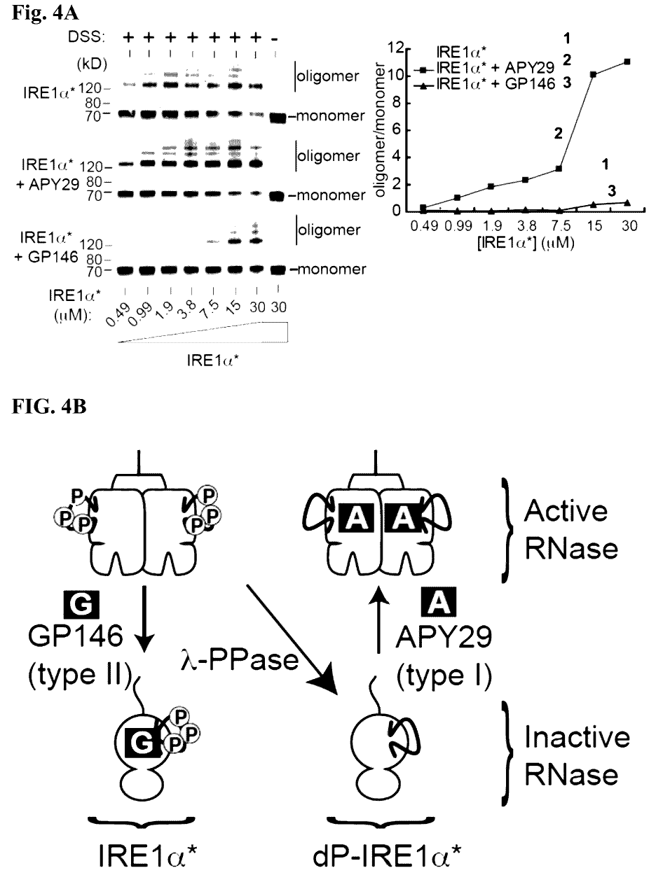

Increasing concentrations of IRE1α* were incubated with either DMSO, or saturating concentrations of APY29 or GP146 and the ratio of oligomeric—defined as all species greater than monomers (mostly dimers)—to monomeric IRE1α was determined (FIG. 4A). In the absence of ligands, IRE1α* shows a concentration-dependent increase in the oligomer/monomer ratio. The presence of APY29 further enhances—whereas GP146 decreases—this concentration-dependent increase in the IRE1α* oligomer/monomer ratio. Taken together, the in vitro data support a model in which these two classes of kinase inhibitors divergently modulate IRE1α* RNase activity by exerting opposing effects on the oligomerization state of the enzyme (FIG. 4B).

6. GP146 Blocks Both the Autophosphorylation and RNase Activities of Endogenous IRE1α In Vivo

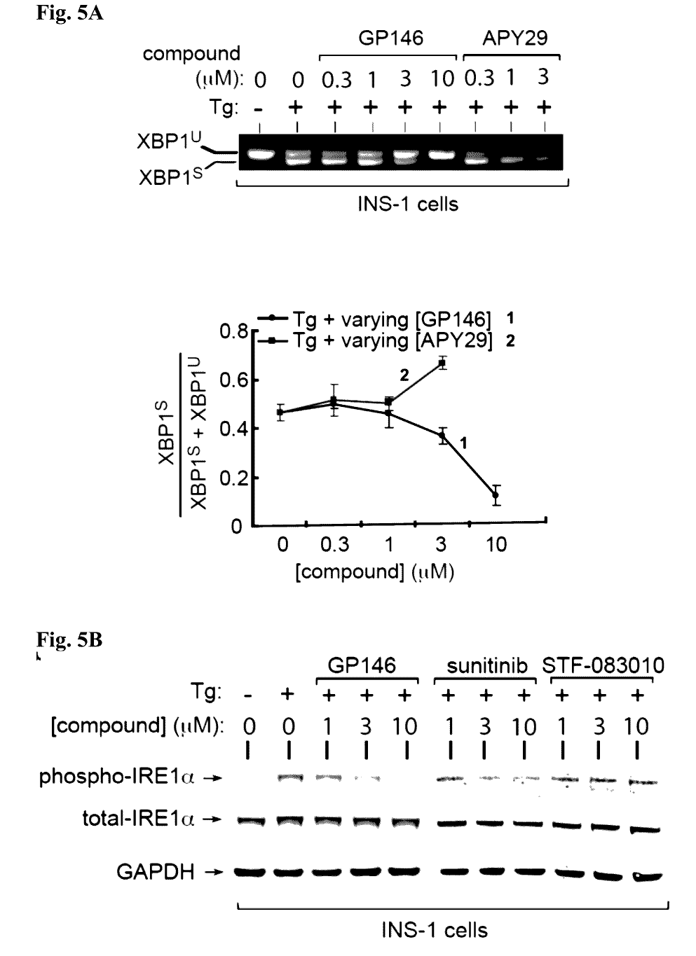

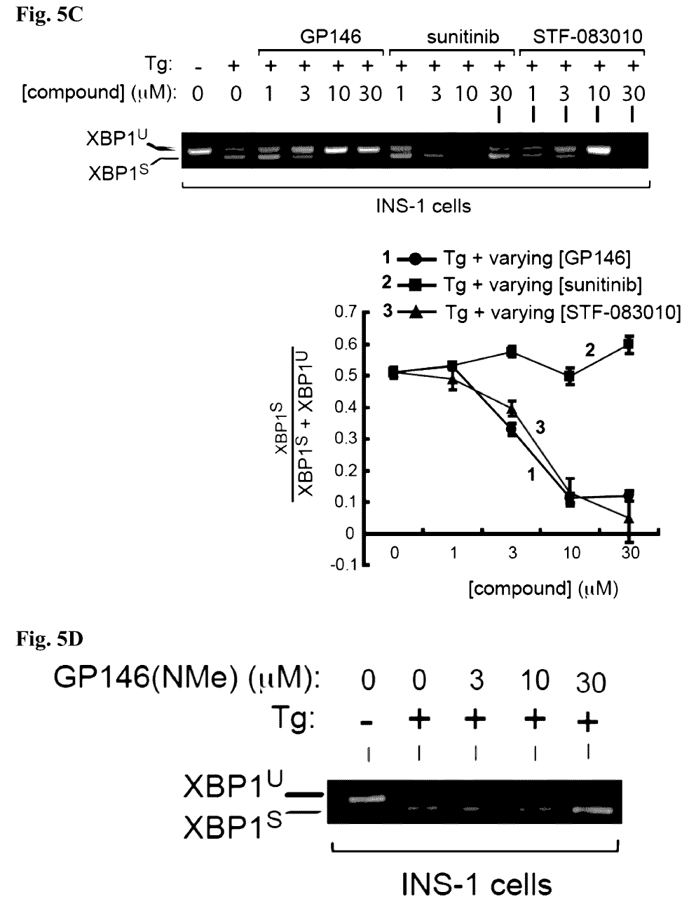

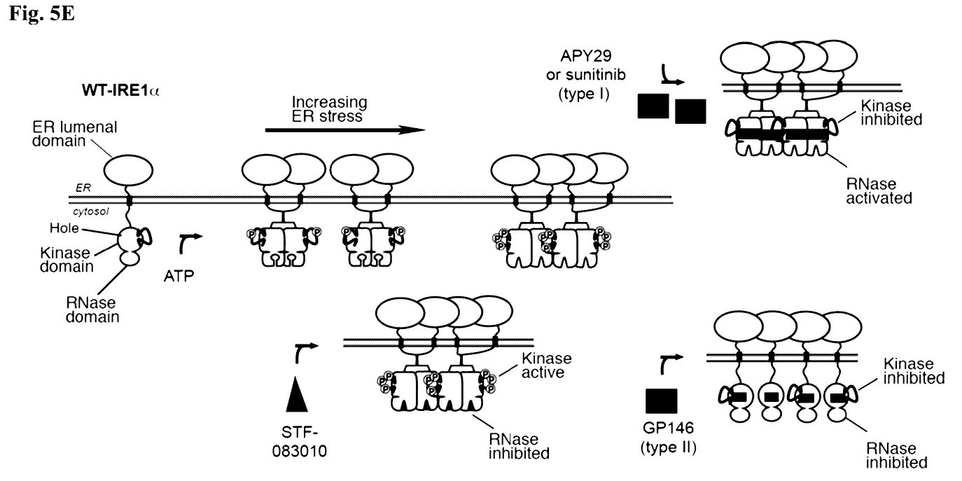

To further explore how IRE1α modulators affect the kinase and RNase activities of endogenous IRE1α under ER stress, in vivo studies using INS-1 rat insulinoma cell lines, which are derived from insulin-producing pancreatic β-cell tumors and contain large well-developed ERs, were conducted. These cells were treated with the ER SERCA ATPase pump inhibitor, thapsigargin (Tg), to induce ER stress and IRE1α activation at levels causing ˜50% splicing of cellular XBP1 mRNA (FIG. 5A). Recapitulating the in vitro results, GP146 and APY29 demonstrate opposing dose-dependent effects on ER stress-induced activation of the RNase of endogenous IRE1α (FIG. 5A). Furthermore, GP146 abrogates IRE1α autophosphorylation at a similar concentration as it blocks RNase activity (FIGS. 5B and 5C). Control compound GP146(NMe) does not block the splicing of XBP1 mRNA (FIG. 5D). Consistent with its in vitro activity, the type I inhibitor sunitinib is able to partially inhibit the kinase activity of IRE1α, but has no effect on the RNase activity of this enzyme (FIGS. 5B and 5C) at the concentrations tested. The RNase inhibitor STF-083010 was also tested in INS-1 cells that had been treated with Tg. As expected, this compound inhibits XBP1 splicing in a dose-dependent manner, but does not prevent IRE1α auto-phosphorylation (FIGS. 5B and 5C). Therefore, GP146 is the only compound identified to date that has the ability to block both enzymatic activities of IRE1α, both in vitro and in vivo (FIG. 5E).

7. In Vitro Characterization of Compounds (IRE1α*)

KIRAs are tested for ability to inhibit IRE1α* kinase and RNase activities in vitro. The XBP1 minisubstrate assay shown in FIGS. 1B-1C, is amenable to 96- or 384-well format, and is used to determine RNase IC₅₀s. In parallel, kinase IC₅₀s for all KIRAs are determined in a 96-well dot blot assay with 32γ-ATP and STK peptide substrate 2 as substrates. Time-dependence of inhibition is determined for all electrophile-containing KIRAs. RNase and kinase assays have been used to profile the KIRAs in FIG. 13, showing a strong correlation between kinase and RNase IC₅₀s. The most potent KIRAs are tested in an IRE1α* autophosphorylation assay. Compounds that exhibit an IC₅₀(RNase)<200 nM are counter-screened against a panel of kinases that are likely off-targets (Src, Abl, p38α), affect cell fate (mTor, IKKβ), down-regulate protein translation (PKR, PERK, GCN2, HRI), are down-stream targets of IRE1 signaling (Jnk1/2, MKK4, MKK7), and are representative of the entire kinome (AurA, Erk2, Cdk2, EGFR, FAK, MAP3K5, PKA). The profiling data obtained from these screens are used to design more selective KIRAs. KIRA3 and KIRA6 have been tested against 12 kinases in this panel and the only off-target inhibition observed is for the tyrosine kinases Src and Abl. Thus, the structure-based design strategy uses these two kinases as counter targets (described below).

8. Molecular Modeling.

Molecular modeling of KIRA interactions with the ATP-binding site of IRE1α: To gain insight into how KIRA3 and KIRA6 interact with the IRE1α ATP-binding site, molecular modeling experiments were used. A model of the DFG-in ATP-binding site conformation was generated from a co-crystal structure of human IRE1α bound to ADP. As a structure of IRE1α in the DFG-out conformation has not yet been described, a homology model of this form was generated by using the activation loop of another kinase, the tyrosine kinase Abl2, as a template. Both the DFG-in and DFG-out models were optimized using multi-step all-atom minimization and explicit water molecular dynamics (MD) simulations. Consistent with the observed SAR and type II pharmacophores of KIRA3 and KIRA6, these ligands are only accommodated in the ATP-binding site of IRE1α when the DFG-motif is in the “out” conformation (DFG-out). Furthermore, the most favorable docking poses for both KIRA3 and KIRA6 involve both of these ligands making all of the canonical contacts of type II inhibitors. Using this model, the predicted docking scores for the KIRAs shown in FIG. 3C are very consistent with in vitro kinase and RNase inhibition results. For example, KIRA7 (R₁=V; R₃=C: IC₅₀(RNase)=<30 nM; IC₅₀(kinase)=35 nM) and KIRA6 (R₁=V; R₃=B: IC₅₀(RNase)=210 nM; IC₅₀(kinase)=620 nM) have significantly and slightly more favorable Glide_SP and MMGBSA scores than KIRA3, respectively. Furthermore, this model has been used to design inactive KIRA3 analogs for controls in cellular assays. Currently, the model for the IRE1α “DFG-out” inactive conformation is used to filter potential analog syntheses based upon their docking scores.

The DFG-in structure of IRE1α was generated from a co-crystal structure of human IRE1α bound to ADP (PDB code 3P23, chain A) (SEQ ID NO:5) (Ali, M. M. et al. EMBO J. 30, 894-905 (2011)). The structure was prepared using the protein preparation workflow in Maestro (Schrodinger) to assign hydrogens, optimize hydrogen bonds, and to perform constraint minimization (impref). The homology model of IRE1α in the DFG-out conformation was built using the activation loop of a DFG-out template kinase (Abl2; PDB code 3GVU) (SEQ ID NO:3) (Salah, E. et al. J. Med. Chem. 54, 2359-2367 (2011)) in Prime (Schrodinger). The initial DFG-out model was optimized using the protein preparation workflow described above. Both DFG-in and DFG-out models were then optimized using a multi-step all-atom minimization and molecular dynamics (MD) simulation implemented in the software package Desmond (DE Shaw Research) (Bowers, K. J. et al. in Proceedings of the ACM IEEE Conference on Supercomputing (SC06) (Tampa, Fla., USA, 2006)). Optimizations were run using the OPLS-AA force field, the TIP4P explicit solvent model in an orthorhombic simulation box 10 Å distance in all directions and adding counter ions. Simulations were performed at 300 K and 1.01325 bar using the NPT ensemble class. All other settings were default. The production simulation time was 12 ns. Simulations were run on an IBM E-server 1350 cluster (36 nodes of 8 Xeon 2.3 GHZ cores and 12 GB of memory). Several later simulation frames were extracted from the DFG-in and DFG-out simulations based on conformational diversity and low (stable) RMSD. These frames were then used to generate the DFG-in and DFG-out models of IRE1α^{I642A}. To avoid side chain clashes, constraint (impref) minimization (in Maestro, Schrodinger) was performed for the WT and I642A structures of IRE1α. These structures were then used for further modeling.

Using the optimized DFG-in and DFG-out structures of WT and IRE1α^{I642A} described above, initial binding poses for APY29 and GP146 were generated as follows. Ligands were prepared using ligprep (Schrodinger Inc.) to generate ionization states (pH=7) and stereoisomers resulting in two representations for GP146 and four for APY29. Ligands were initially docked into the DFG-in and DFG-out models of IRE1α using the Induced Fit Docking (IFD) (Schrodinger Inc.) protocol with default settings. The IFD protocol includes a constraint receptor minimization step followed by initial flexible Glide docking of the ligand using a softened potential to generate an ensemble of poses. For each pose, the nearby receptor structure was then refined using Prime. Each ligand was then re-docked (using Glide) into its corresponding optimized low-energy receptor structure and ranked by Glide score. The best pose with highest IFD score obtained for each ligand was again subjected to MD simulation (8-10 ns production runs) for further optimization of the protein ligand complex. The MD protocol includes a multi-step procedure for minimizations and short MD runs followed by the production MD simulation. Ligand poses were observed to be stable during the production MD runs. The final frames of these simulations were then used for ligand docking after constraint (impref) minimization (Maestro, Schrodinger Inc.). The best pose for each ligand was selected based on the Glide score, known interactions and visual inspection. The 3D plots of ligand poses were produced using PyMol.

9. Duration and Magnitude of ER Stress Determine Entry into the Terminal UPR Through High-Order Oligomerization and Hyperactivation of IRE1α Kinase/RNase Catalytic Domains.

To rigorously test for cytoprotective effects, thresholds of ER stress were dilenated that when crossed push cells into apoptosis. Using rat insulinoma (INS-1) cells that have a well-developed ER and secrete insulin, the duration and magnitude of ER stress that triggers apoptosis were quantified. Two variables—concentration of ER stress inducer (e.g. Thapsigargin—Tg) and time of exposure to the agent—are directly linked to the percentage of cells entering apoptosis. Such regimes can be defined for other ER stress inducers such as the glycosylation inhibitor, tunicamycin (Tm). Increasing ER stress causes progressive increases in endogenous IRE1α phosphorylation, increases in XBP1 mRNA splicing, depletion through endonucleolytic decay of the ER-localized mRNA, Ins1, which encodes proinsulin, and induction of the pro-apoptotic transcription factor, CHOP.

These aforementioned terminal UPR signaling events can be completely mimicked simply by controlled over-expression of IRE1α, in the complete absence of upstream ER stress. Tetracycline-inducible isogenic INS-1 stable cell lines expressing WT IRE1α were generated. Induced with doxycycline (dox) the transgenic IRE1α proteins oligomerize, spontaneously trans-autophosphorylate and trigger XBP1 mRNA splicing. The transgenic systems are finely tuneable, with increasing [Dox] causing progressively greater IRE1α induction (the transgenic IRE1α protein is Myc-tagged). Phospho/Myc IRE1α ratios are finely controllable (and measureable) with increasing [Dox], as is XBP1 mRNA splicing. Mimicking dose-dependency by ER stress agents into a terminal UPR, increasing [Dox] spontaneously triggers entry into a terminal UPR by causing IRE1α kinase hyper-phosphorylation and RNase hyperactivation past a critical threshold and induction of key signature events of the terminal UPR. These include reduction of miR-17 and Ins1 mRNA, induction of CHOP mRNA, induction and proteolytic cleavage of caspases 1,2, and 3, and apoptosis as measured by Annexin-V staining.

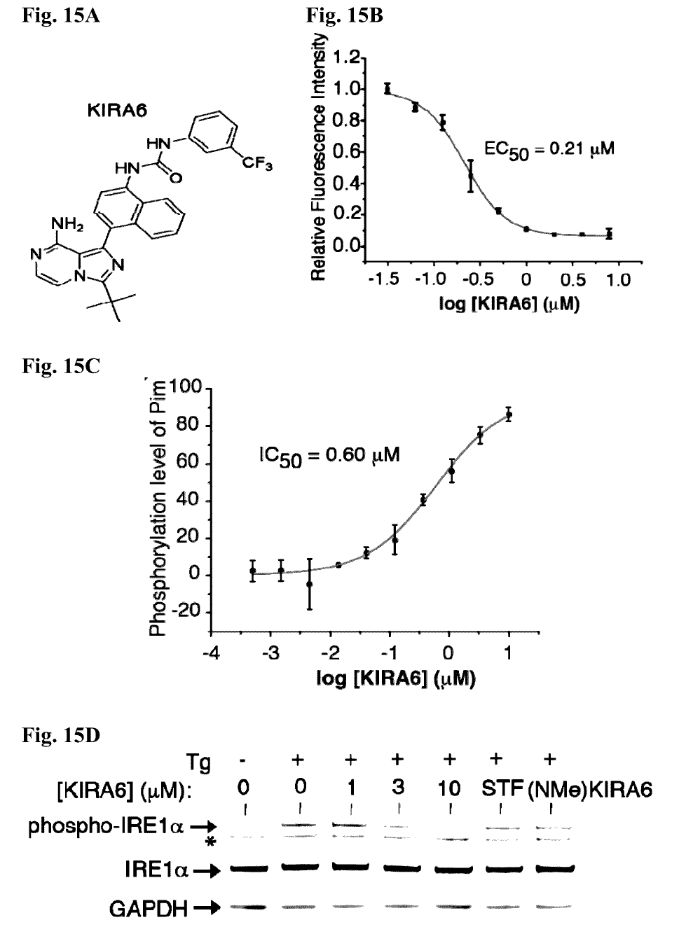

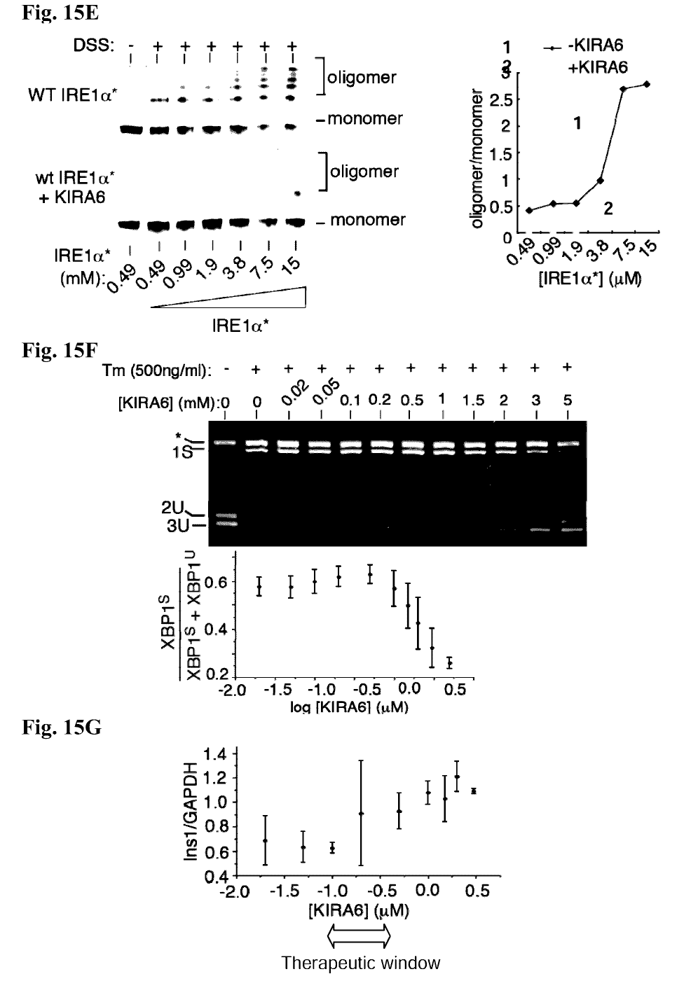

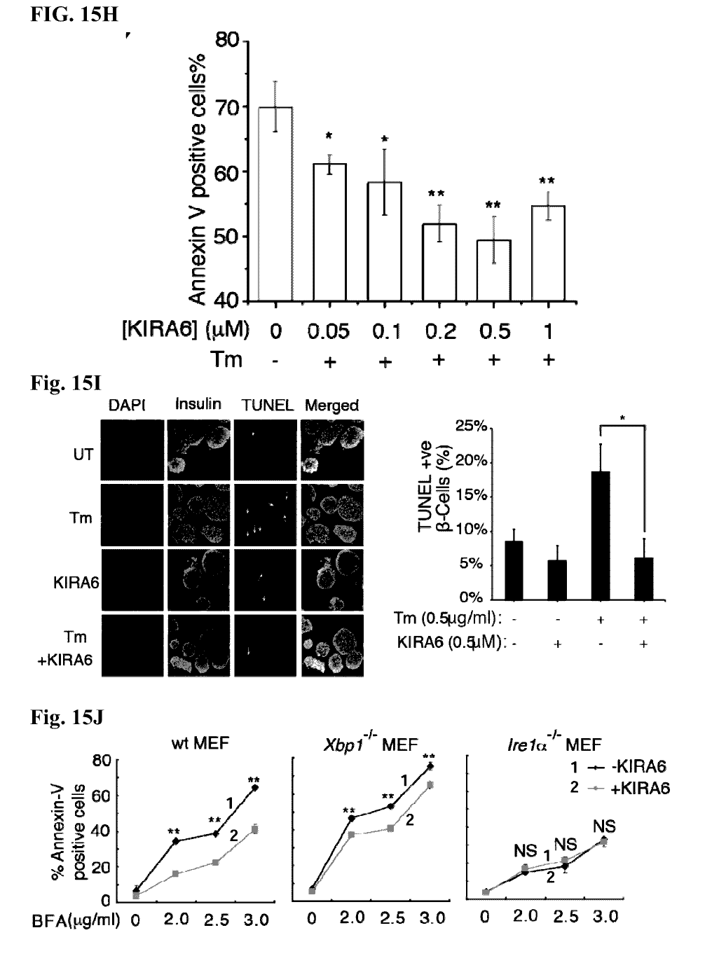

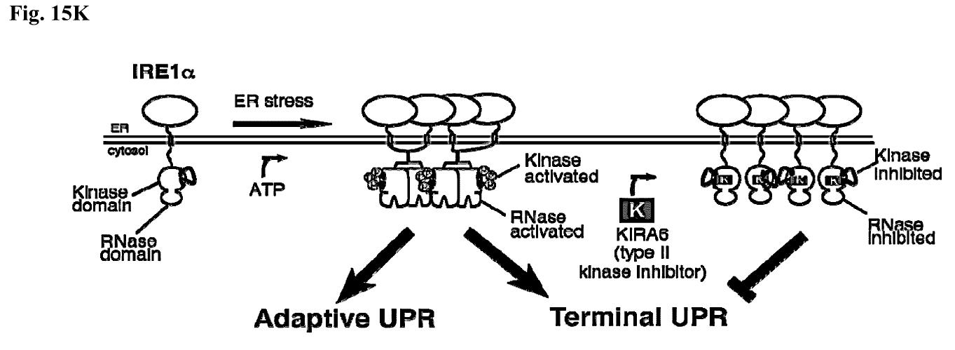

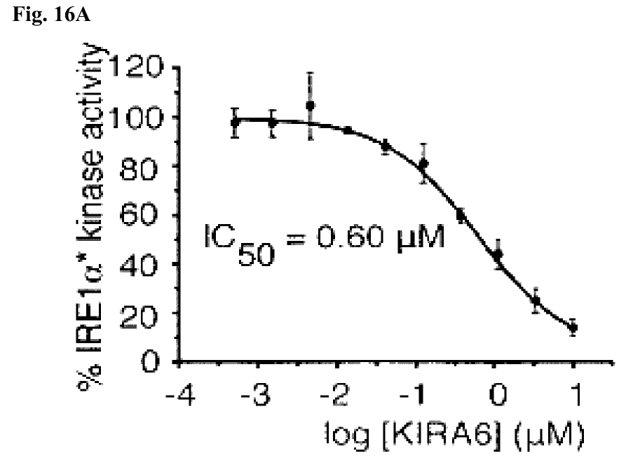

KIRAs break high-order oligomerization of IRE1α kinase domains, attenuate RNAse activity, and reduce entry of cells into the Terminal UPR. In exciting preliminary data, all these terminal UPR endpoints are curtailed by pre-treating cells with KIRA6, before exposing them to ER stress inducers. in vitro, KIRA6 inhibits IRE1α* RNase and kinase activities with similar IC₅₀s (FIGS. 15B and 15C). in vivo, KIRA6 inhibits endogenous IRE1α auto-phosphorylation in a dose-dependent manner (FIG. 15D); in contrast the aldehyde-based IRE1α RNase-inhibitor, STF, does not inhibit IRE1α auto-phosphorylation, nor does a control compound KIRA6(in).

Furthermore, in vitro, KIRA6 reduces concentration-dependent oligomerization of IRE1α* (FIG. 15E). in vivo, KIRA6 inhibits endogenous IRE1α—mediated XBP1 mRNA splicing provoked by Tm (FIG. 15F). Intriguingly, KIRA6 inhibits ER-localized endonucleolytic decay of Ins1 mRNA at lower doses of the drug than needed to inhibit XBP1 mRNA splicing (FIG. 15G); this discriminatory effect may occur because higher-order oligomers are needed to catalyze Ins1 mRNA decay, whereas dimers suffice for XBP1 mRNA splicing. Because ER-localized endonucleolytic mRNA decay promotes apoptosis—in contrast to XBP1 mRNA splicing, which promotes adaptation—this differential effect of KIRAs reveals the existence of a natural “therapeutic window” which reduces entry of INS1-1 cells into apoptosis (FIG. 15H). These cytoprotective effects of KIRA6 extend to myriad cell types, including freshly harvested pancreatic islets from C57/BL6 mice treated with Tm (FIG. 15I). KIRA6 cytoprotective effects are dependent on IRE1α because they are absent in Ire1α^{-/-} mouse embryonic fibroblasts (MEFs), but still demonstrable in WT and Xbp1^{-/-} MEFs (FIG. 15J). A model of KIRA6-mediated cytoprotection is shown (FIG. 15K); it posits that the type II kinase inhibitor, KIRA6, reduces kinase/RNAse homo-oligomerization on the cytosolic face of IRE1α, which consequently reduces RNase hyperactivity under ER stress, preventing pro-apoptotic ER-localized mRNA decay and granting cells extended reprieve from programmed cell death.

10. Allosteric Modulation of the IRE1α RNase with Novel Kinase Inhibitor.

KIRA6, a more potent version of earlier KIRAs, whose structure is shown in FIG. 15A has been developed. This compound dose-dependently reduces kinase autophosphorylation and XBP1 splicing activity of IRE1α* (WT) (FIGS. 15B-15C). In addition, KIRA6 dose-dependently inhibits IRE1α* (WT) cleavage of pre-miR-17.

11. KIRA6 Inhibits IRE1α In Vivo to Preserve Cell Viability and Function in Diverse Cells and Rodent Tissues Experiencing ER Stress.

Additional experiments focused on testing physiological effects of IRE1α kinase inhibition. APY29 showed pleiotropic toxicity, including proliferative blocks at low micromolar concentrations, precluding further in vivo testing of ON-target effects. In contrast, KIRA6 had negligible toxicity up to 10 μM, providing a favorable therapeutic index to test cytoprotection. The INS-1 lines confirmed ON-target effects: Pro-Caspase-3 cleavage upon IRE1α (WT) expression is prevented by KIRA6. Moreover, despite its inability to directly inhibit JNK activity in vitro, KIRA6 strongly inhibits JNK phosphorylation from IRE1α hyperactivation or ER stress. Also, KIRA6 dose-dependently inhibits Ins1 mRNA decay, proinsulin depletion, and apoptosis from IRE1α hyperactivation.

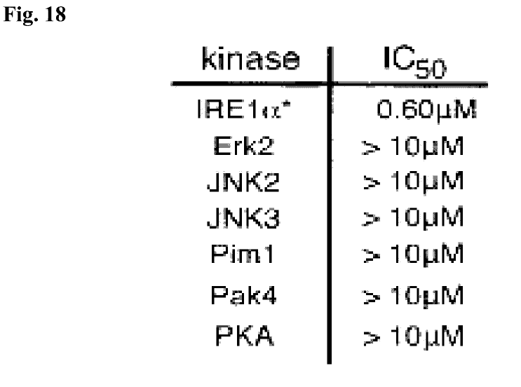

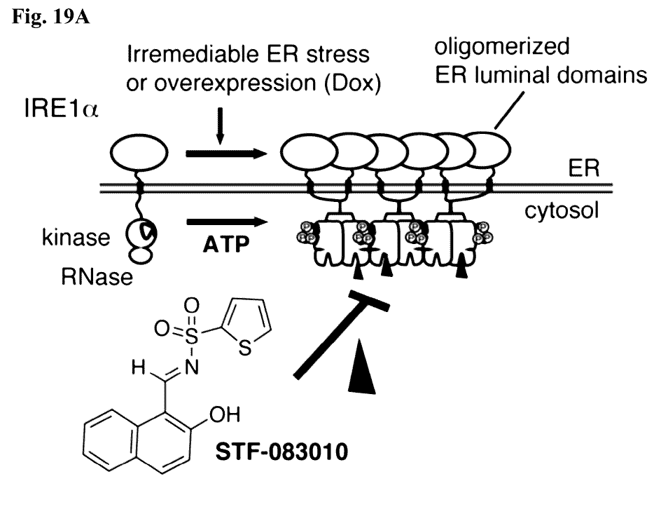

Chemical-genetic tools enabled ON-target competition tests. KIRA6: [1] reduces 1NM-PP1-induced XBP1 RNA cleavage by IRE1α* (I642G) in vitro; [2] antagonizes 1NM-PP1-induced XBP1 splicing by IRE1α (I642G) in vivo; and [3] reduces 1NM-PP1 potentiation of Ins1 mRNA decay and apoptosis during ER stress, in dose-dependent manner. KIRA6 does not inhibit the activity of a panel of Ser/Thr kinases (including JNK2 and 3) in vitro (FIG. 18). Moreover, KIRA6 does not inhibit nor secondarily promote eIF2α phosphorylation by PERK, the other UPR kinase.

Having confirmed that KIRA6 has ON-target effects, we next tested efficacy against endogenous IRE1α using the established ER stress regimes in their linear ranges straddling the apoptotic trigger point. In INS-1 cells, KIRA6 inhibits IRE1α auto-phosphorylation by Tg and XBP1 mRNA splicing by Tm in a dose-dependent manner; whereas, a control analog, (NMe)KIRA6, inhibits neither output at 10 μM.

We next tested multiple Terminal UPR endpoints and found that KIRA6: [1] Inhibits Ins1 and Ins2 mRNA decay by Tm in INS-1 cells in dose-dependent manner. We noted that the in vivo IC₅₀ of KIRA6 for Ins1 mRNA rescue is lower than that for inhibiting XBP1 splicing, and Ins2 mRNA levels recover even at 20 nM KIRA6 and exceed basal, untreated levels in dose-dependent manner. Furthermore, KIRA6: [2] Inhibits TXNIP induction by Tm in murine C57BL/6 pancreatic islets; [3] Inhibits IRE1α-dependent activation of a TXNIP 3′UTR luciferase reporter containing its two miR-17 binding sites; [4] Prevents 1L-13 secretion by Tm and Tg (but not ATP) in THP1 macrophage lines; [5] Prevents loss of INS-1 Ki67-positive cells and C57BL/6 pancreatic islet double-positive Nkx6.1/EdU β-cells under ER stress; [6] Inhibits apoptosis of INS-1 cells under BFA in dose-dependent manner; [7] Reduces TUNEL staining of β-cells in C57BL/6 and human islets under Tm; and [8] preserves glucose-stimulated insulin secretion (GSIS) in C57BL/6 islets under Tm.

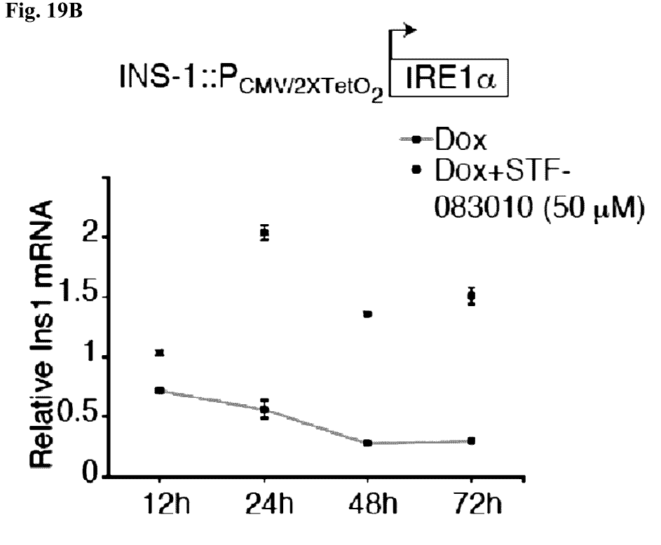

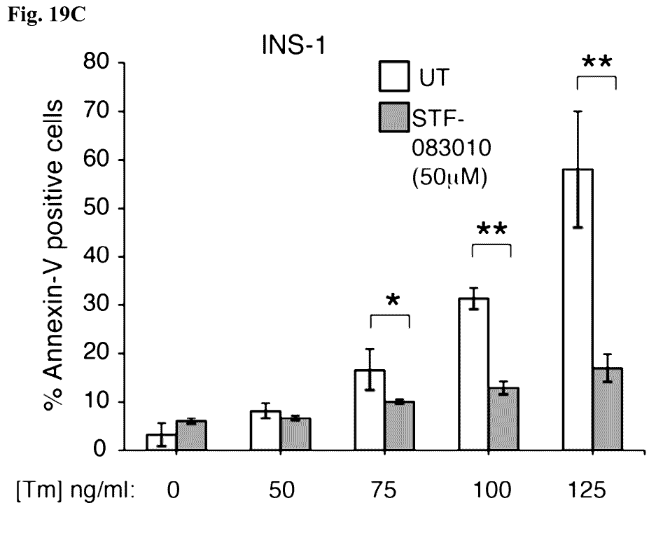

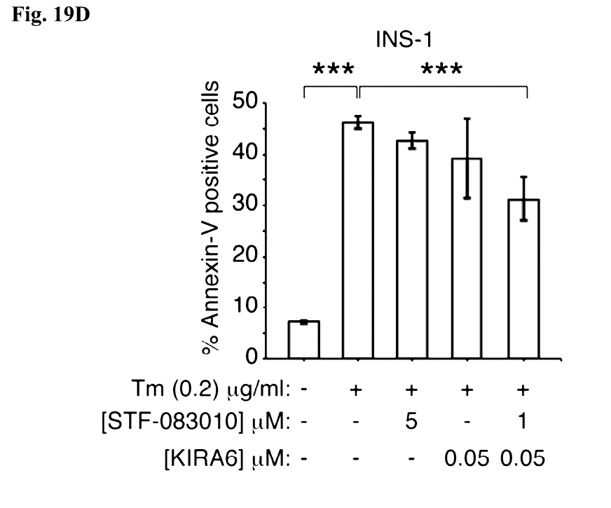

We also tested effects of STF-083010, a small molecule compound that reactively modifies Lysine 907 in the RNase active site through an aldehyde intermediate (Papandreou et al., 2011)(FIG. 19A). As with KIRA6, STF-083010 (at 50 M) also decreases Ins1 mRNA decay under IRE1α hyperactivation and apoptosis by Tm (FIGS. 19B-19C). Moreover, when used in combination at doses that are sub-therapeutic individually, STF-083010 (1 M) and KIRA6 (50 nM) afford significant cytoprotection under Tm (FIG. 19D). Together, these data further implicate IRE1α's RNase in promoting apoptosis, in this case by showing that the RNase can even be inhibited combinatorially through two distinct sites in IRE1α for cytoprotection.

To rule out the possibility that KIRA6 defeats ER stress agents upstream of IRE1α, we tested whether blocks to ER post-translational modification still persist under KIRA6. A test substrate, the null Hong Kong variant of alpha-1 anti-trypsin (NHK-A1AT), normally glycosylated and ER-retained, is deglycoslyated under Tm. NHK-A1AT clearly remains deglycoslyated under both Tm and KIRA6.

Encouraged by clear and convincing evidence that KIRA6 preserves cell viability and function in multiple cell and explant systems under diverse ER stress regimes, we next applied a higher evidentiary standard by testing disease-relevant animal models. Given compelling evidence that ER stress contributes to photoreceptor loss in many retinal diseases, including retinitis pigmentosa (RP) (Zhang et al., 2014), we tested KIRA6 in two rodent models. Transgenic rats expressing a misfolded Rhodopsin mutant (P23H) exhibit spontaneous photoreceptor degeneration and are used as a model of autosomal dominant RP (Gorbatyuk et al., 2010). Retinas of hemizygous P23H rats develop normally but lose photoreceptors beginning on postnatal day (P) 10; by P40, the outer nuclear layer (ONL), representing photoreceptor nuclei, is reduced to ˜50% of the thickness of wild-type rats (Pennesi et al., 2008). We intravitreally injected KIRA6 or an equivalent volume of carrier into either eye of individual P23H rats at P9 and P15. ONL thickness at P40 revealed partial, yet statistically significant, protection from photoreceptor loss in KIRA6-treated eyes.

Given rapid clearance of intravitreally injected small molecules (half-life<60 h), we employed a model of acute photoreceptor loss occurring over 7 days from a single intravitreal injection of Tm into adult rats (Shimazawa et al., 2007). Intravitreal co-injection of KIRA6 with Tm significantly reduces XBP1 splicing, TXNIP induction, and decay of the ER-localized photoreceptor-specific Rhodopsin mRNA. Rhodopsin mRNA may be an IRE1α RNase substrate since Rhodopsin RNA is cleaved by IRE1α*, but not RNase-dead IRE1α* (N906A), at a primary G/C site with flanking sequence similarity to scission sites in XBP1. KIRA6 inhibits Rhodopsin RNA cleavage by IRE1α* in dose-dependent manner. Concomitant with blockage of Terminal UPR outputs, co-injection of KIRA6 in the Tm model reduces photoreceptor loss as assessed by Optical Coherence Tomography (OCT) and histology.

Next, to test whether KIRA6 also provides functional protection, we established a dose-response curve to determine threshold [Tm] that cause functional retinal damage as measured by scotopic electroretinograms (ERG). Based on the results, we injected Tm at 3 μg/ml. In this regime, co-injection with KIRA6 substantially protects against loss of ERG responsiveness, significantly preserving both a- and b-wave amplitudes.

Finally, to test in vivo efficacy of systemic KIRA6, we chose the Ins2^{+/Akita} (Akita) mouse, which expresses a mutant (C96Y) proinsulin unable to complete oxidative folding, thus causing chronic ER stress, β-cell apoptosis, and diabetes in the first few weeks of life (Lerner et al., 2012). Pharmacokinetic profile of KIRA6 in BALB/c mice intraperitoneally (i.p.) dosed at 10 mg/kg showed good drug plasma AUC levels (AUC 0-24 h=14.3 μM*h) with moderate clearance (22.4 mL/min/kg). Drug half-life was 3.90 hours, Cₘₐₓ was 3.3 μM, and plasma levels at 4 and 8 hr were 1.2 μM and 0.33 μM, respectively. Initial systemic studies utilized a Tm i.p. challenge in C57BL/6 mice, with and without KIRA6 co-injection, and UPR markers measured in liver. Low dose Tm (2 μg/kg) elevates liver XBP1 splicing without decay of ER-localized Blos 1 mRNA (Hollien et al., 2009), while KIRA6 co-provision reduces XBP1 splicing. Escalation of Tm to 100 μg/kg further increases XBP1 splicing and triggers Blos 1 mRNA decay, with both markers attenuated by KIRA6.

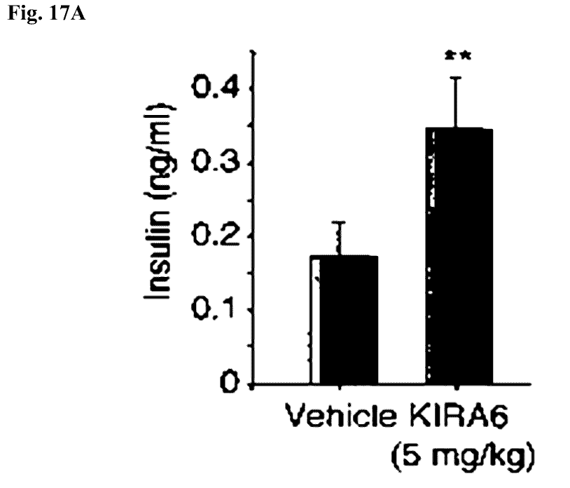

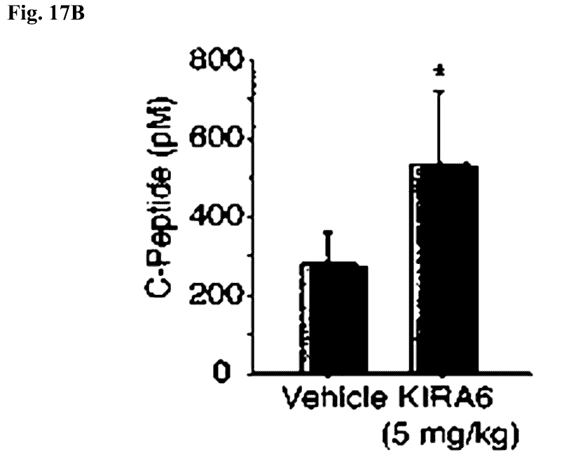

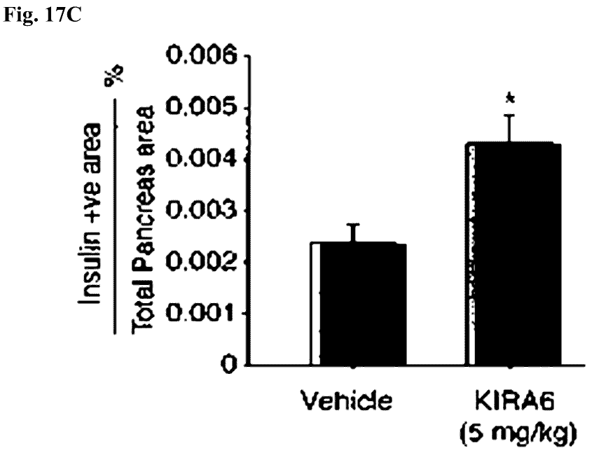

Based on low micromolar KIRA6 needed for protection in cell culture, we chose i.p. dosing regimens of 5 or 10 mg/kg b.i.d for Akita chronic efficacy studies to provide similar exposure. We injected KIRA6 into randomized 3-week old male Akita mice when their random blood glucose levels were at prediabetic range (˜200 mg/dl). In both dosing regimes, we observed significant amelioration of random glucose levels over several weeks in KIRA6-treated mice compared to vehicle, both fed ad lib. TXNIP mRNA levels decline in islets of KIRA6-treated mice within one week, without compensatory increase of CHOP mRNA (downstream of PERK). KIRA6-treated mice appeared healthy even after 49 days from initial injection and displayed no significant differences in weight from vehicles. Even 12 days after stopping injections, the 5 mg/kg KIRA6-treated mice show significantly improved random blood glucose levels and glucose tolerance tests (GTT). Even 21 days after stopping injections, KIRA6-treated mice display statistically significant doubling in both plasma insulin and C-peptide levels (FIGS. 17A-17B). H&E and insulin staining of whole pancreas sections reveals increased islet size in KIRA6 treated-animals. Insulin-positive islet areas remained significantly higher in the KIRA6-treated group 18 days after stopping injections (FIG. 17C).

A class of IRE1α inhibitors that disrupt oligomerization should reduce RNase activity and Terminal UPR events in tandem. Unique among multi-domain kinases, the mechanistic relationship between IRE1α's kinase and RNase allows divergent small molecule allosteric control (Wang et al., 2012). Whereas both are ATP-competitive, IRE1α type I kinase inhibitors increase oligomerization to increase RNase activity, while IRE1α type II kinase inhibitors decrease oligomerization to decrease RNase activity. Here we developed and tested the effects of KIRA6, a novel IRE1α type II kinase inhibitor. Given that KIRA6 has a favorable therapeutic index and shows IRE1α ON-target effects, we tested whether it would reduce cell death under ER stress. Remarkably, blocking IRE1α with KIRA6 raises the apoptotic threshold and enhances survival during ongoing upstream ER stress, indicating that destructive signaling rather than a compromised ER micro-environment per se promotes cell death. While poly-pharmacological toxicity precluded testing ON-target effects of APY29, our results justify development and testing of non-toxic type I kinase inhibitors against IRE1α mutants.

12. Tissue Culture

INS-1 cells with doxycycline (Dox)-inducible expression of wild-type and mutant mouse IRE1α were grown in RPMI, 10% fetal bovine serum, 1 mM sodium pyruvate, 10 mM HEPES, 2 mM glutamine, 50 M β-mercaptoethanol, as described previously (Han et al., 2009). To generate the Dox-inducible IRE1α human (WT) and IRE1α human cancer mutant cell lines, INS-1/FRT/TO cells were grown in the above media with 10 μg/ml blasticidin. Cells were then grown in 200 μg/ml zeocin, cotransfected with 1 g pcDNA5/FRT/TO:IRE1α human (WT), (L474R), (R635W), (S769F), (Q780*) and (P830L) mutant constructs and 1 μg FLP recombinase (pOG44) using Lipofectamine (Invitrogen). After 4 hrs, cells were switched to zeocin-free media, trypsinized 48 hrs later, and then plated in media containing hygromycin (150 g/ml), which was replaced every 3 days until colonies appeared. SV40-transformed MEFs were maintained in Dulbecco's Modified Eagle Media (DMEM) containing 5 mM glucose and supplemented with 10% heat-inactivated fetal bovine serum. Thapsigargin (Tg), Brefeldin A (BFA), and Dox were purchased from Sigma-Aldrich. Tunicamycin (Tm) was purchased from Millipore. APY29, KIRA6, 1NM-PP1 and (NMe)KIRA6 were synthesized in house.

13. Western Blots and Antibodies

For protein analysis, cells were lysed in M-PER buffer (Thermo Scientific) plus complete EDTA-free protease inhibitor (Roche) and phosphatase inhibitor cocktail (Sigma). The concentration of samples was determined using BCA Protein Assay (Thermo). Western blots were performed using 10% and 12% Bis-Tris precast gels (NuPage) on Invitrogen XCell SureLock® Mini-Cell modules. Gels were run using MES buffer and transferred onto nitrocellulose transfer membrane using an XCell II™ Blot Module. Blocking, antibody incubation, and washing were done in PBS or TBS with 0.05% Tween-20 (v/v) and 5% (w/v) non-fat dry milk or BSA, or blocking buffer (Licor-Odyssey). Antibodies used were: mouse anti-Actin (Sigma-Aldrich); rabbit anti-cleaved Caspase-2 (Abcam); mouse anti-GAPDH, anti-c-Myc, anti-rabbit, anti-proinsulin, and anti-IRE1α (Santa Cruz Biotechnology); anti-phospho-IRE1α and anti-full length Caspase-2 (Novus Biologicals); and rabbit anti-Caspase-3, anti-JNK, anti-eIF2a, anti-phospho-eIF2a, and mouse anti-phospho-JNK (Cell Signaling). Antibody binding was detected by using near-infrared-dye-conjugated secondary antibodies (Licor) on the LI-COR Odyssey scanner or visualized by capturing on a CL-XPosure film using ECL SuperSignal West Dura Extended Duration Substrate (both from Thermo Scientific).

14. RNA Isolation, Real-Time PCR (Q-PCR), and Primers

RNA was isolated from whole cells using either Qiagen RNeasy kits or Trizol (Invitrogen). TissueLyser II (Qiagen) was used for RNA isolation from liver and retina. Primers used for Q-PCR were as follows: Rat TXNIP: 5′-CTGATGGAGGCACAGTGAGA-3′ (SEQ ID NO:6) and 5′-CTCGGGTGGAGTGCTTAGAG-3′ (SEQ ID NO:7); rat GAPDH 5′-AGTTCAACGGCACAGTCAAG-3′ (SEQ ID NO:8) and 5′-ACTCAGCACCAGCATCACC-3′ (SEQ ID NO:9); rat Ins1, 5′-GTCCTCTGGGAGCCCAAG-3′ (SEQ ID NO:10) and 5′-ACAGAGCCTCCACCAGG-3′ (SEQ ID NO: 11); rat p21, 5′-TGAACCGCTGTCTTGAGATG-3′ (SEQ ID NO: 12) and 5′-TCTTGGTTGCCTCTTTTGCT-3′ (SEQ ID NO:13); mouse Blosl, 5′-CAAGGAGCTGCAGGAGAAGA-3′ (SEQ ID NO: 14) and 5′-GCCTGGTTGAAGTTCTCCAC-3′ (SEQ ID NO:15); mouse Beta-Actin, 5′-GCAAGTGCTTCTAGGCGGAC-3′ (SEQ ID NO:16) and 5′-AAGAAAGGGTGTAAAACGCAGC-3′ (SEQ ID NO: 17). For standard mRNA, 1 g total RNA was reverse transcribed using the QuantiTect Reverse Transcription Kit (Qiagen). For Q-PCR, we used SYBR green (Qiagen) and StepOnePlus Real-Time PCR System (Applied Biosystems). Thermal cycles were: 5 min at 95° C., 40 cycles of 15 s at 95° C., 30 s at 60° C. Gene expression levels were normalized to GAPDH or Actin. For TaqMan Q-PCR, cDNA was produced using a target-specific probe, TaqMan Universal PCR Master Mix, the TaqMan microRNA reverse transcription kit (both Applied Biosystems) and the Bio-Rad iCycler Thermal cycler. For both regular Q-PCR and TaqMan Q-PCR, the reactions were performed on C1000 thermal cycler and measurements were recorded on a CFX96 Real-Time PCR Detection System (both from Bio-Rad). The following targeted primers and probes sets were used in TaqMan Q-PCR: snoRNA135, RPL21 and hsa-miR-17 (all from Applied Biosystems).

15. XBP-1 mRNA Splicing

RNA was isolated from whole cells or tissues and reverse transcribed as above to obtain total cDNA. Then, XBP-1 primers were used to amplify an XBP-1 amplicon spanning the 26 nt intron from the cDNA samples in a regular 3-step PCR. Thermal cycles were: 5 min at 95° C., 30 cycles of 30 s at 95° C., 30 s at 60° C., and 1 min at 72° C., followed by 72° C. for 15 min, and a 4° C. hold. Sense primer rat XBP1.3S (5′-AAACAGAGTAGCACAGACTGC-3′) (SEQ ID NO: 18) and antisense primer rat XBP1.2AS (5′-GGATCTCTAAGACTAGAGGCTTGGTG-3′) (SEQ ID NO: 19) were used. PCR fragments were then digested by PstI, resolved on 2% agarose gels, stained with EtBr and quantified by densitometry using ImageJ (U. S. National Institutes of Health). Spliced XBP1 was also determined in mouse liver by Q-PCR using mouse XBP1 sense (5′-AGGAAACTGAAAAACAGAGTAGCAGC-3′) (SEQ ID NO:20) and antisense (5′-TCCTTCTGGGTAGACCTCTGG-3′) (SEQ ID NO:21) primers.

16. Flow Cytometry

For assaying apoptosis by Annexin V staining, cells were plated in 12-well plates overnight. Cells were then treated with various reagents for indicated time periods. On the day of flow cytometry analysis, cells were trypsinized and washed in PBS and resuspended in Annexin V binding buffer with Annexin-V FITC (BD Pharmingen™). Flow cytometry was performed on a Becton Dickinson LSRII flow cytometer.

17. Islet Staining

Islets were extracted from C57BL/6 mice using previously reported methods (Szot et al., 2007), and cultured in RPMI+10% FBS with 0.5 μg/mL Tm with or without KIRA6 (0.5 μM) or left untreated for 16 hrs. Approximately 150 islets were cultured for each condition in triplicate. Non-diabetic human islets were obtained from Prodo Labs (Irvine, Calif.) and cultured in Prodo Islet Medium (PIM from Prodo Labs). For analysis of non-ER stress treated conditions, islets were cultured in PIM for 16 hrs before harvesting. Islets were then spun, washed once with PBS, and fixed for 30 min with 4% PFA. After fixation, islets were washed twice with PBS, followed by a wash in 100% ethanol. After removal of all ethanol, 100 μL of prewarmed Histogel (Thermo Scientific) was added to the eppendorf tube and placed at 4° C. to solidify before paraffin embedding and 5 m sectioning of the islets. Islets were stained with TUNEL using ApopTag® Red In Situ Apoptosis Detection Kit (Millipore) according to the manufacturer's instructions. Islets were also co-stained with anti-TXNIP (MBL International), guinea pig anti-insulin (Zymed), DAPI (Sigma), and goat anti-guinea pig secondary (Rockland) before mounting onto slides with VectaShield (Vector Laboratories). At least 10 islets and >500 β-cell nuclei were counted per group, in triplicate. Cells were considered TUNEL positive if staining was present and colocalized with DAPI staining, indicating nuclear localization.

18. MTT Assay

INS-1 CAT, IRE1α human (WT) and IRE1α human mutant cells were seeded at 40% confluence in a 96-well plate and treated or not treated with indicated concentrations of Tg or Dox (1 μg/ml). At the indicated time points, medium was replaced with 100 μl of 1 mg/mL 3-(4,5-dimethylthiazol-2-yl)-2,5-diphenyltetrazolium bromide (MTT) (Molecular Probes) in RPMI. After incubation at 37° C. for 4 hrs, 75 μl MTT medium was removed, and 50 μl DMSO were added to dissolve precipitate. Absorbance was recorded at 540 nm using a Spectramax M5 microplate reader (Molecular Devices).

19. Superfolder GFP-IRE1α Construction and Microscopy

The first 27 amino acids of mouse IRE1α containing the signal peptide were cloned just before the first ATG of sfGFP lacking the stop codon, and the rest of the sequence of mouse IRE1α (WT) or IRE1α (I642G) was cloned in frame after the sfGFP in a pCDNA5 FRT/TO mammalian expression plasmid. INS1-FRT/TO cells were transfected as described previously (Han et al., 2009) to generate stable cell lines expressing the above constructs. After induction of INS1-sfGFP-IRE1α cells for 24 hrs with Ing/ml Dox (a sub-apoptotic dose sufficient for imaging the reporter), live cells were imaged on a widefield microscope after a further treatment with 1 mM DTT in the presence or absence of 1 μM KIRA6 (Axiovert 200; Carl Zeiss MicroImaging, Inc.) with a 63X/1.4 NA oil objective and a 450-490 nm excitation/500-550 emission bandpass filter using a Retiga 2000R camera. Composite figures were prepared using ImageJ (NIH), Photoshop CS4 and Illustrator CS4 software (Adobe). For INS1-sfGFP-IRE1α(I642G) cells, 5 μM 1NM-PP1 was used for induction in presence of Ing/ml Dox for 12 h. Live cells were imaged (Nikon Eclipse Ti-Yokogawa CSU22 spinning disk confocal) with Apo 100X/1.49 oil objective and a 491 nm excitation/525-50 emission filter. Figures were prepared using ImageJ (NIH).

20. Null Hong Kong α-1 Antitrypsin De-Glycosylation Immunoblots

pCDNA3.1-α1 hAT-NHK plasmid expressing the NHK-α1AT, was transfected into HEK293 cells. Cells were then treated with Tm plus/minus KIRA6 to check protein glycosylation status. NHK-α1AT remains glycosylated under normal conditions producing a protein band at ˜50 KDa by immunoblot. However, tunicamycin (glycosylation inhibitor) treatment inhibits glycosylation and the deglycosylated band appears at ˜42 KDa. Goat anti-human α1-antitrypsin antibody (MP biomedicals) was used for detection by immunoblot.

21. Detection of IL1-β

Human THP-1 cells were grown in RPMI-1640 with 10% FBS and 50 μM 2-mercaptoethanol, differentiated for 2 hrs with 0.5 M phorbol-12-myristate-13-acetate (Sigma), and primed for 18 hrs with 1 μg/ml ultrapure lipopolysaccharide (LPS;Sigma). Cell culture media was changed to media without LPS and treated with 0.5 μM KIRA6 for 2 hrs prior to the addition of 5 mM ATP (Roche), or 1 M Tg or 10 μg/ml Tm. After 4 hrs incubation, media supernatant was collected and assayed for hIL-1β by ELISA (Thermo Scientific).

22. TXNIP 3′UTR Reporter Luciferase Assay

Luciferase reporter containing TXNIP 3′ UTR with miR-17 binding sites was used as described (Lemer et al., 2012). To measure luciferase activity a Dual-Glo (Promega #E2920) kit was used as per manufacturer's instructions. The luciferase enzyme activity was detected on a Spectramax M5 microplate reader (Molecular Devices).

23. Glucose-Stimulated Insulin Secretion (GSIS) Assay

Freshly isolated islets from 9-week-old C57BL/6 mice were cultured in RPMI-1640 with 10% FCS, 2 mM L-glutamine, 0.1 mM 2-mercaptoethanol, and 11 mM glucose with or without Tm (500 ng/ml) for 16 hrs before the GSIS assay. KIRA6 (0.5 uM) was added 2 hrs before treating with Tm. In the GSIS assay, islets were preincubated in HEPES-buffered Krebs-Ringer bicarbonate solution (KRBH) (10 mM HEPES [pH 7.4], 129 mM NaCl, 4.7 mM KCl, 1.2 mM KH₂PO₄, 1.2 mM MgSO₄, 2 mM CaCl₂, 5 mM NaHCO₃, and 0.1% BSA) containing 2.5 mM glucose for 30 min at 37° C. Thirty islets per condition were incubated with either 2.5 mM or 16.7 mM glucose in KRBH at 37° C. for 60 min. Collected media were analyzed by anti-insulin ELISA (EMD Millipore).

24. Image Guided Optical Coherence Tomography (OCT)

Mice were anaesthetized with 1.5-3% isoflurane, eyes were dilated with 2.5% phenylephrine hydrochloride and 1% tropicamide, and corneas were kept moist with regular application of 2.5% methylcellulose. Eyes were examined using a Micron III retinal imaging system (Phoenix Research Labs). Spectral domain OCT images were acquired with a Micron Image Guided OCT System (Phoenix Research Labs) by averaging 10 to 50 scans.

25. Electroretinography (ERG)

Rats dark-adapted overnight were anesthetized with ketamine (100 mg/ml) and xylazine (20 mg/ml) via intraperitoneal injection in dim red light. Pupils were dilated with 2.5% phenylephrine hydrochloride and 1.0% tropicamide, and corneas were anesthetized with 0.5% proparacaine. Scotopic ERG recordings were performed as previously described⁴. Briefly, 10-sec flashes of white light of increasing intensities were used to induce bilateral, full-field ERGs; responses were recorded using contact lens electrodes with a UTAS-E 3000 Visual Electrodiagnostic System (LKC Technologies, Inc.). Electroretinography (ERG) Rats dark-adapted overnight were anesthetized with ketamine (100 mg/ml) and xylazine (20 mg/ml) via intraperitoneal injection in dim red light. Pupils were dilated with 2.5% phenylephrine hydrochloride and 1.0% tropicamide, and corneas were anesthetized with 0.5% proparacaine. Scotopic ERG recordings were performed as previously described⁴. Briefly, 10-μsec flashes of white light of increasing intensities were used to induce bilateral, full-field ERGs; responses were recorded using contact lens electrodes with a UTAS-E 3000 Visual Electrodiagnostic System (LKC Technologies, Inc.).

26. Islet Culture and Proliferation Assessment (EdU Staining)

C57BL/6 mouse islets were isolated as previously described (Szot et al., 2007), cultured for 5 days, followed by 2 days of indicated treatment. DMSO was utilized as control, Tm at 0.5 ug/ml, and KIRA6 at 1 μm. After 48 hrs, islets were treated with 10 mM 5-Ethynyl-2-deoxyuridine (EdU) for 3 hrs, and then immediately fixed in 4% paraformaldehyde/10 mM PBS solution for 25 min. Islets were washed 3 times with 10 mM PBS for 20 min, permeabilized with 0.3% TritonX-100 in 10 mM PBS for 3 hrs, then blocked in 5% goat serum/0.15% Triton-X 100/10 mM PBS overnight at 4° C. and washed twice with antibody dilution buffer for 15 min at room temperature. Primary antibody, rabbit anti-human NKX6.1 1:500 (Sigma-Aldrich), and secondary antibody, Cy3 conjugated goat anti-rabbit 1:500 (Sigma), were diluted in 1% BSA/0.2% Triton X-100/10 mM PBS and incubated for 24 hrs at 4° C. Click-iT® EdU Alexa Fluor® Imaging Kit (Invitrogen) was used to identify dividing cells after immunostaining. Islets were mounted with Fluoromount™ (Sigma) and imaged using a Leica SP5 confocal laser scanning microscope (Leica). The Volocity software (PerkinElmer) colocalization macro was utilized to quantify dual EdU and the unique 3-cell nuclear marker Nkx6.1.

27. Animal Analytic Studies

C567BL/6 and C57BL/6 Ins2^{WT/C96Y} (Ins2^{+/Akita}) mice were obtained from Jackson Laboratories. Glucose levels were measured from tail snips obtained between 9:00 and 11:00 AM using a glucometer (Nova Statstrip Xpress, Data Sciences International). Serum insulin and C-peptide levels were measured using mouse ultra-sensitive insulin and C-peptide ELISA (Mercodia). Animals were maintained in a specific pathogen-free animal facility on a 12-h light-dark cycle at an ambient temperature of 21° C. They were given free access to water and food. All experiments used age-matched male mice.

28. Ins2^{+/Akita} Mouse Genotyping

Akita mouse colonies were maintained and genotyped as described previously (Lemer et al., 2012).

29. Glucose Tolerance Test

Mice were fasted for 17 h before i.p. injection with glucose (2 g/kg of body weight). Blood was collected from the tail, and glucose levels were determined by using a glucometer (Nova Statstrip Xpress, Data Sciences International).

30. Mouse Injections

Male Ins2^{+/Akita} mice were injected intraperitoneally with KIRA6 in a 2 mg/ml solution made of 3% Ethanol: 7% Tween-80: 90% Saline twice a day (b.i.d). Same solution without KIRA6 is denoted as Vehicle. C567BL/6 mice were also injected with same KIRA6 solution and indicated doses of Tm for liver analysis.

31. Pancreatic Insulin-Positive β-Cell Area Determination

Pancreatic sectioning, staining and analysis were done as described previously (Chintinne et al., 2010). Briefly, whole pancreas in paraffin-embedded blocks from vehicle (n=3) and KIRA6 (n=6) treated mice were serially sectioned at intervals of 250 m. Every 10th section was stained with anti-insulin (Invitrogen). Nuclei were stained with DAPI (Sigma-Aldrich). A minimum of 2% of the total organ volume was stained and measured. The pancreas and β-cell areas were measured with the Zeiss Axiolmager Brightfield Microscope and quantified with VOLOCITY software.

32. Rat Husbandry

All studies and procedures were performed in accordance with the guidelines of the Institutional Animal Care and Use Committee at the University of California, San Francisco, and in compliance with the ARVO Statement for the Use of Animals in Ophthalmic and Vision Research. P23H rhodopsin transgenic rats (line 1) were described previously (www.ucsfeye.net/mlavailRDratmodels.shtml); wild-type Sprague-Dawley (SD) rats served as controls. All animals were housed in barrier facility free of specific pathogens on a 12 hrs light/dark cycle with food and water available ad libitum.

33. KIRA6 and KIRA6_NMe Synthesis.

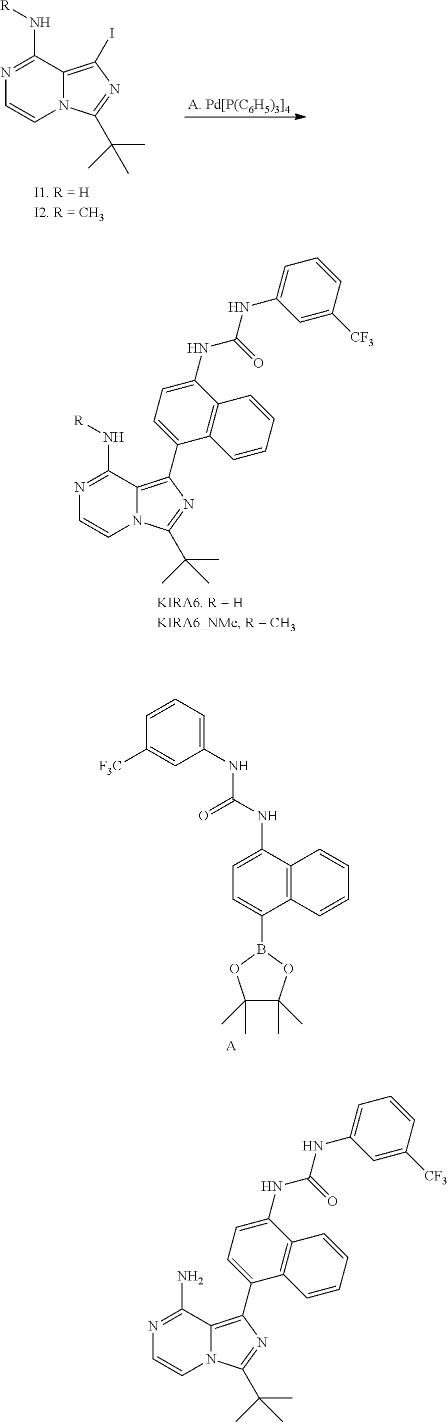

KIRA6. 1-(4-(8-amino-3-tert-butylimidazo[1,5-a]pyrazin-1-yl)naphthalen-1-yl)-3-(3-(trifluoromethyl)phenyl)urea (KIRA6). A mixture of I1 (60.0 mg, 0.120 mmol), A (66 mg, 0.15 mmol), tetrakis(triphenylphosphine)palladium (5 mg, 4 μmol) and sodium carbonate (928 mg, 0.27 mmol) was dissolved in a 3:1 mixture of DME/water (0.5 mL). The mixture was heated overnight at 85° C. The crude mixture was cooled to room temperature, diluted in a mixture of acetonitrile/water and purified by reverse phase chromatography (HPLC) to obtain 53 mg of KIRA6. TLC (CH₂Cl₂:MeOH, 95:5 v/v): R_{f}=0.4; ¹H NMR (300 MHz, MeOD): δ 8.26 (m, 1H), 8.08-7.99 (m, 2H), 7.90-7.86 (m, 1H), 7.83-7.79 (m, 1H), 7.69-7.52 (m, 5H), 7.35 (d, J=7.4 Hz, 1H), 6.98 (m, 1H), 1.65 (s, 9H); ¹³C NMR (126 MHz, MeOD): δ 154.8, 140.2, 135.7, 133.0, 132.4, 131.7, 131.6, 131.0, 130.7, 129.4, 128.8, 128.6, 128.5, 127.0, 126.6, 125.9, 125.4, 123.2, 121.9, 120.1, 118.7, 115.0, 114.4, 110.1, 33.6, 27.3; ESI-MS (m/z): [M]+ calcd. for C₂₈H₂₅F₃N₆O (M+H⁺): 519.2; found 519.4. The purity of KIRA6 was determined with two analytical RP-HPLC methods, using a Varian Microsorb-MV 100-5 C18 column (4.6 mm×150 mm), and eluted with either H₂O/CH₃CN or H₂O/MeOH gradient solvent systems (+0.05% TFA) run over 30 min. Products were detected by UV at 254 nm. KIRA6 was found to be >95% pure in both solvent systems.

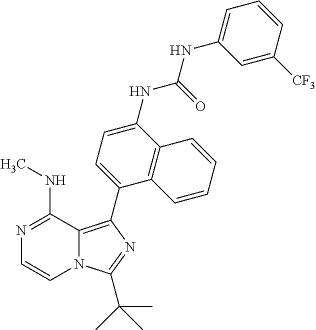 [SMILES: CNc1nccn2c(C(C)(C)C)nc(-c3ccc(NC(=O)Nc4cccc(C(F)(F)F)c4)c4ccccc34)c12]

### (NMe)KIRA6.

1-(4-(8-methylamino-3-tert-butylimidazo[1,5-a]pyrazin-1-yl)naphthalen-1-yl)-3-(3-(trifluoromethyl)phenyl)urea ((NMe)KIRA6). A mixture of 12 (14.6 mg, 0.044 mmol), A (22 mg, 0.048 mmol), tetrakis(triphenylphosphine)palladium (2.0 mg, 1.73 μmol) and sodium carbonate (13.6 mg, 0.128 mmol) was dissolved in a 3:1 mixture of DME/water (0.22 mL). The mixture was heated overnight at 85° C. The crude mixture was cooled to room temperature, diluted in a mixture of acetonitrile/water and purified by reverse phase chromatography (HPLC) to obtain 10.2 mg of (NMe)KIRA6. TLC (CH₂Cl₂:MeOH, 95:5 v/v): R_{f}=0.4; 1H NMR (300 MHz, Chloroform-d) δ 9.06 (m, 1H), 8.63 (m, 1H), 8.19 (d, J=6.7 Hz, 1H), 8.05 (m, 1H), 7.68 (s, 1H), 7.56-7.46 (m, 3H), 7.18 (m, 3H), 7.01-6.95 (m, 1H), 3.08 (s, 3H), 1.58 (s, 9H); ESI-MS (m/z): [M]+ calcd. for C₂₉H₂₇F₃N₆O (M+H⁺): 533.2; found 533.6. The purity of (NMe)KIRA6 was determined with two analytical RP-HPLC methods, using a Varian Microsorb-MV 100-5 C18 column (4.6 mm×150 mm), and eluted with either H₂O/CH₃CN or H₂O/MeOH gradient solvent systems (+0.05% TFA) run over 30 min. Products were detected by UV at 254 nm. (NMe)KIRA6 was found to be >95% pure in both solvent systems.

34. In Vitro IRE1α* Protein Preparation, Crosslinking, RNase and Kinase Assay.

A construct containing the cytosolic kinase and RNase domains of human IRE1α (residues 469-977, IRE1α*) or equivalent IRE1α mutants was expressed in SF9 insect cells using the Bac-to-Bac baculovirus expression system (Invitrogen) as described (Wang et al., 2012). X-PPase (NEB)-treated dephosphorylated IRE1α* (dP-IRE1α*) was also prepared and all the crosslinking experiments were performed as described (Wang et al., 2012). The RNase assay using 5′FAM-3′BHQ-labeled XBP1 single stem-loop minisubstrate (5′FAM-CUGAGUCCGCAGCACUCAG-3′BHQ, from Dharmacon) (SEQ ID NO:22), and the IRE1α*auto-phosphorylation kinase assay were both done as described (Wang et al., 2012). The ability of KIRA6 to inhibit the catalytic activity of Erk2, JNK2, JNK3, PKA, and Piml was determined using previously reported assay conditions (Hill et al., 2012). Internally ³²P-labelled RNAs (mouse XBP1 RNA and Ins2 RNA, as described in (Han et al., 2009), and rat rhodopsin mRNA) were also used as substrates. For time-course determination, IRE1α* proteins (16 nM) were incubated with or without 10 μM 1NM-PP1 for 20 min prior to addition of 20 nM radio-labeled RNAs. Reactions were quenched by addition of 4 M urea at different time points and then were subjected to urea 6% PAGE analysis. For the endpoint readings of APY29-mediated rescue of IRE1α* P830L RNase activity, 160 nM of IRE1α* (wt or P830L mutant) was incubated with 20 μM APY29 and mixed with 13 nM radio-labeled mouse Ins2 RNA for 1 hr reaction and subsequently resolved by urea 6% PAGE. Determination of KIRA6-mediated inhibition of wt IRE1α* was done by incubating 16 nM IRE1α* and 20 nM radio-labeled mouse Ins2 RNA or 0.8 μM IRE1α* and 40 nM radio-labeled miR17 as described (Upton et al., 2012), or 0.33 μM IRE1α* and 20 nM radio-labeled rat rhodopsin mRNA. The cleavage buffer and general manipulation are the same as described (Wang et al., 2012). cDNA for rat rhodopsin was purchased from Invitrogen and amplified by PCR using primers: 5′-GAAATTAATACGACTCACTATAGGGGGTCCAGGTACATCCCCGAG-3′ (forward) (SEQ ID NO:23) and 5′-TTAGGCTGGAGCCACCTGGCT-3′ (reverse) (SEQ ID NO:24). RNA was in vitro transcribed, cleaved by IRE1α*, and the cleavage sites mapped as described (Wang et al., 2012).

| SEQ ID NO: 1 | |
|---|---|
| MSYYHHHHHHDYDIPTTENLYFQGAMDPE hq qqqlqhqqfq | 480 |
| kelekiqllq qqqqqlpfhp pgdtaqdgel ldtsgpyses sgtsspstsp rasnhslcsg | 540 |
| ssaskagssp sleqddgdee tsvvivgkis fcpkdvlghg aegtivyrgm fdnrdvavkr | 600 |
| ilpecfsfad revqllresd ehpnviryfc tekdrqfqyi aielcaatlq eyveqkdfah | 660 |
| lglepitllq qttsglahlh slnivhrdlk phnilismpn ahgkikamis dfglckklav | 720 |
| grhsfsrrsg vpgtegwiap emlsedcken ptytvdifsa gcvfyyvise gshpfgkslq | 780 |
| rqanillgac sldclhpekh edviarelie kmiamdpqkr psakhvlkhp ffwslekqlq | 840 |
| ffqdvsdrie kesldgpivk qlerggravv kmdwrenitv plqtdlrkfr tykggsvrdl | 900 |
| lramrnkkhh yrelpaevre tlgslpddfv cyftsrfphl lahtyramel csherlfqpy | 960 |
| yfheppepqp pvtpdal | |
| SEQ ID NO: 2 | |
| hq qqqlqhqqfq | 480 |
| kelekiqllq qqqqqlpfhp pgdtaqdgel ldtsgpyses sgtsspstsp rasnhslcsg | 540 |
| ssaskagssp sleqddgdee tsvvivgkis fcpkdvlghg aegtivyrgm fdnrdvavkr | 600 |
| ilpecfsfad revqllresd ehpnviryfc tekdrqfqyi aielcaatlq eyveqkdfah | 660 |
| lglepitllq qttsglahlh slnivhrdlk phnilismpn ahgkikamis dfglckklav | 720 |
| grhsfsrrsg vpgtegwiap emlsedcken ptytvdifsa gcvfyyvise gshpfgkslq | 780 |
| rqanillgac sldclhpekh edviarelie kmiamdpqkr psakhvlkhp ffwslekqlq | 840 |
| ffqdvsdrie kesldgpivk qlerggravv kmdwrenitv plqtdlrkfr tykggsvrdl | 900 |
| lramrnkkhh yrelpaevre tlgslpddfv cyftsrfphl lahtyramel csherlfqpy | 960 |
| yfheppepqp pvtpdal | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a disease in a patient in need of such treatment, said method comprising administering a first amount of an Ire1 kinase modulating compound, or a pharmaceutically acceptable salt thereof, and a second amount of an Ire1 ribonuclease modulating compound, or a pharmaceutically acceptable salt thereof, to said patient, wherein the disease is a neurodegenerative disease, demyelinating disease, cancer, eye disease, fibrotic disease, or diabetes, wherein the first amount and the second amount are together a combined synergistic amount.

2. The method of claim 1, wherein the neurodegenerative disease is retinitis pigmentosa, amyotrophic lateral sclerosis, retinal degeneration, macular degeneration, Parkinson's Disease, Alzheimer Disease, Huntington's Disease, Prion Disease, Creutzfeldt-Jakob Disease, or Kuru.

3. The method of claim 1, wherein the demyelinating disease is Wolfram Syndrome, Pelizaeus-Merzbacher Disease, Transverse Myelitis, Charcot-Marie-Tooth Disease, or Multiple Sclerosis.

4. The method of claim 1, wherein the eye disease is retinitis pigmentosa, retinal degeneration, macular degeneration, or Wolfram Syndrome.

5. The method of claim 1, wherein the fibrotic disease is idiopathic pulmonary fibrosis (IPF), myocardial infarction, cardiac hypertrophy, heart failure, cirrhosis, acetominophen (Tylenol) liver toxicity, hepatitis C liver disease, hepatosteatosis (fatty liver disease), or hepatic fibrosis.

6. The method of claim 1, wherein said Ire1 kinase modulating compound has the formula: 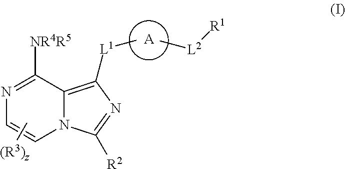
wherein,
ring A is substituted or unsubstituted cycloalkylene, substituted or unsubstituted heterocycloalkylene, substituted or unsubstituted arylene, or substituted or unsubstituted heteroarylene;
L¹ is a bond or unsubstituted C₁-C₅ alkylene;
L² is a bond, —NR^{6a}—, —O—, —S—, —C(O)—, —S(O)—, —S(O)₂—, —NR^{6a}C(O)—, —C(O)NR^{6b}—, —C(O)(CH₂)_{z2}—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently hydrogen, oxo, halogen, —CX^{b}₃, —CN, —SO₂Cl, —SOₙ₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ and R⁵ are independently hydrogen or unsubstituted C₁-C₆ alkyl;
R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl;
each occurrence of the symbols n, n1, and n2 is independently an integer from 0 to 4;
each occurrence of the symbols m, m1, m2, v, v1, and v2 is independently an integer from 1 to 2;
the symbol z is an integer from 0 to 2;
the symbol z2 is an integer from 1 to 4;
each occurrence of the symbols X, X^{a}, and X^{b} is independently a halogen.

7. The method of claim 6, wherein the Ire1 kinase modulating compound has the formula: 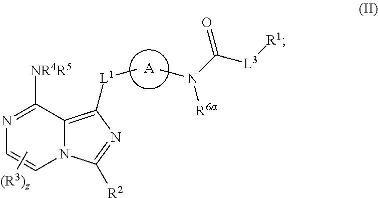
and
L³ is a bond, —NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

8-18. (canceled)

19. The method of claim 6, wherein the Ire1 kinase modulating compound is selected from the group consisting of:

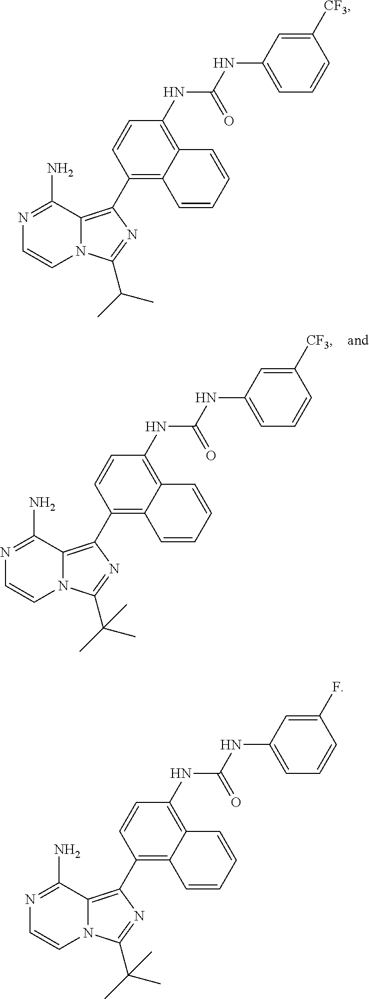 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "and" ; CC(C)(C)c1nc(-c2ccc(NC(=O)Nc3cccc(F)c3)c3ccccc23)c2c(N)nccn12]

20. (canceled)

21. (canceled)

22. The method of claim 1, wherein said Ire1 ribonuclease modulating compound is STF-083010, MKC-3946, 4μ8C, 3-methoxy-6-bromosalicylaldehyde, a salicylaldehyde, or toyocamycin.

23. (canceled)

24. (canceled)

25. A method of modulating the activity of an Ire1 protein, said method comprising contacting said Ire1 protein with an Ire1 kinase modulating compound, or a pharmaceutically acceptable salt thereof, and an Ire1 ribonuclease modulating compound, or a pharmaceutically acceptable salt thereof, wherein said Ire 1 kinase is contacted with a combined synergistic amount of said Ire1 kinase modulating compound and said Ire1 ribonuclease modulating compound.

26. The method claim 25, wherein a cell or an organism comprises said Ire1 protein.

27. (canceled)

28. The method of claim 26, wherein said organism has a disease associated with said Ire1 protein activity.

29. The method of claim 25, wherein said disease is a neurodegenerative disease, demyelinating disease, cancer, eye disease, fibrotic disease, or diabetes.

30. The method claim 25, wherein said Ire1 kinase modulating compound has the formula: 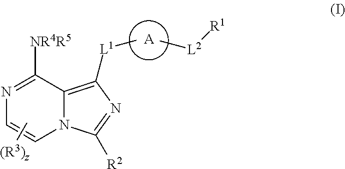
wherein,
ring A is substituted or unsubstituted cycloalkylene, substituted or unsubstituted heterocycloalkylene, substituted or unsubstituted arylene, or substituted or unsubstituted heteroarylene;
L¹ is a bond or unsubstituted C₁-C₅ alkylene;
L² is a bond, —NR^{6a}—, —O—, —S—, —C(O)—, —S(O)—, —S(O)₂—, —NR^{6a}C(O)—, —C(O)NR^{6b}—, —C(O)(CH₂)_{z2}—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently hydrogen, oxo, halogen, —CX^{b}₃, —CN, —SO₂Cl, —SOₙ₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{10b}, —NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ and R⁵ are independently hydrogen or unsubstituted C₁-C₆ alkyl;
R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl;
each occurrence of the symbols n, n1, and n2 is independently an integer from 0 to 4;
each occurrence of the symbols m, m1, m2, v, v1, and v2 is independently an integer from 1 to 2;
the symbol z is an integer from 0 to 2;
the symbol z2 is an integer from 1 to 4;
each occurrence of the symbols X, X^{a}, and X^{b} is independently a halogen.

31. The method of claim 30, wherein the Ire1 kinase modulating compound is selected from the group consisting of:

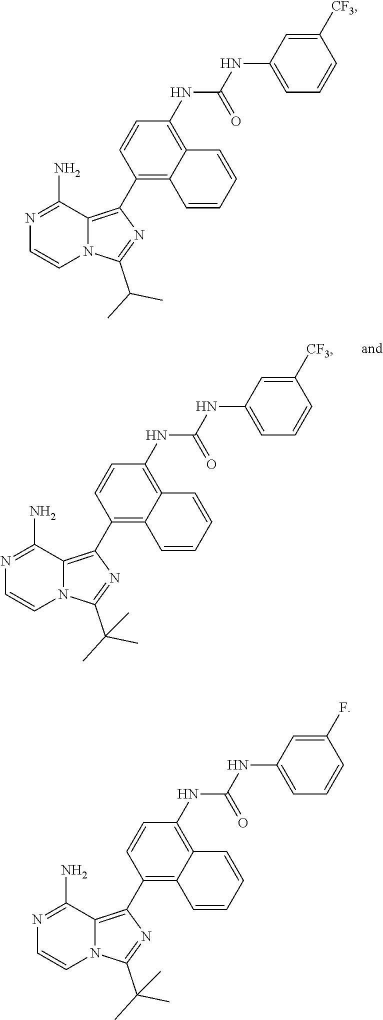 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(F)c3)c3ccccc23)c2c(N)nccn12]

32. (canceled)

33. (canceled)

34. The method of claim 25, wherein said Ire1 ribonuclease modulating compound is STF-083010, MKC-3946, 4μ8C, 3-methoxy-6-bromosalicylaldehyde, a salicylaldehyde, or toyocamycin.

35. (canceled)

36. (canceled)

37. (canceled)

38. A pharmaceutical combination comprising a first amount of an Ire1 kinase modulating compound and a second amount of an Ire1 ribonuclease modulating compound, wherein the first amount and the second amount are together a combined synergistic amount.

39. The pharmaceutical composition of claim 38, wherein said Ire1 kinase modulating compound has the formula: 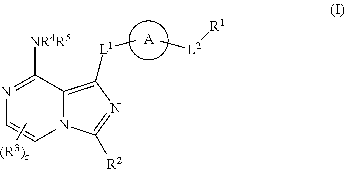
wherein,
ring A is substituted or unsubstituted cycloalkylene, substituted or unsubstituted heterocycloalkylene, substituted or unsubstituted arylene, or substituted or unsubstituted heteroarylene;
L¹ is a bond or unsubstituted C₁-C₅ alkylene;
L² is a bond, —NR^{6a}—, —O—, —S—, —C(O)—, —S(O)—, —S(O)₂—, —NR^{6a}C(O)—, —C(O)NR^{6b}—, —C(O)(CH₂)_{z2}—, —NR^{6a}C(O)O—, —NR^{6a}C(O)NR^{6b}—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, oxo, halogen, —CX₃, —CN, —SO₂Cl, —SOₙR¹⁰, —SOᵥNR⁷R⁸, —NHNH₂, —ONR⁷R⁸, —NHC═(O)NHNH₂, —NHC═(O)NR⁷R⁸, —N(O)ₘ, —NR⁷R⁸, —C(O)R⁹, —C(O)—OR⁹, —C(O)NR⁷R⁸, —OR¹⁰, —NR⁷SOₙR¹⁰, —NR⁷C═(O)R⁹, —NR⁷C(O)OR⁹, —NR⁷OR⁹, —OCX₃, —OCHX₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, oxo, halogen, —CX^{a}₃, —CN, —SO₂Cl, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)OR^{9a}, —C(O)NR^{7a}R^{8a}, —OR^{10a}, —NR^{7a}SOₙ₁R^{10a}, —NR^{7a}C═(O)R^{9a}, —NR^{7a}C(O)OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently hydrogen, oxo, halogen, —CX^{b}₃, —CN, —SO₂Cl, —SOₙ₂R^{10b}, —SOᵥ₂NR^{7b}R^{8b}, —NHNH₂, —ONR^{7b}R^{8b}, —NHC═(O)NHNH₂, —NHC═(O)NR^{7b}R^{8b}, —N(O)ₘ₂, —NR^{7b}R^{8b}, —C(O)R^{9b}, —C(O)—OR^{9b}, —C(O)NR^{7b}R^{8b}, —OR^{b}, NR^{7b}SOₙ₂R^{10b}, —NR^{7b}C═(O)R^{9b}, —NR^{7b}C(O)OR^{9b}, —NR^{7b}OR^{9b}, —OCX^{b}₃, —OCHX^{b}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ and R⁵ are independently hydrogen or unsubstituted C₁-C₆ alkyl;
R⁷, R⁸, R⁹, R¹⁰, R^{6a}, R^{7a}, R^{8a}, R^{9a}, R^{10a}, R^{6b}, R^{7b}, R^{8b}, R^{9b} and R^{10b} are independently hydrogen, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ and R⁸ substituents bonded to the same nitrogen atom may optionally be joined to form a substituted or unsubstituted heterocycloalkyl or substituted or unsubstituted heteroaryl; R^{7a} and R^{8a} substituents bonded to the same nitrogen atom may optionally be joined to form a substituted or unsubstituted heterocycloalkyl or substituted or unsubstituted heteroaryl; R^{7b} and R^{8b} substituents bonded to the same nitrogen atom may optionally be joined to form a substituted or unsubstituted heterocycloalkyl or substituted or unsubstituted heteroaryl;
each occurrence of the symbols n, n1, and n2 is independently an integer from 0 to 4;
each occurrence of the symbols m, m1, m2, v, v1, and v2 is independently an integer from 1 to 2;
the symbol z is an integer from 0 to 2;
the symbol z2 is an integer from 1 to 4;
each occurrence of the symbols X, X^{a}, and X^{b} is independently a halogen.

40. The pharmaceutical composition of claim 39, wherein the Ire1 kinase modulating compound is selected from the group consisting of:

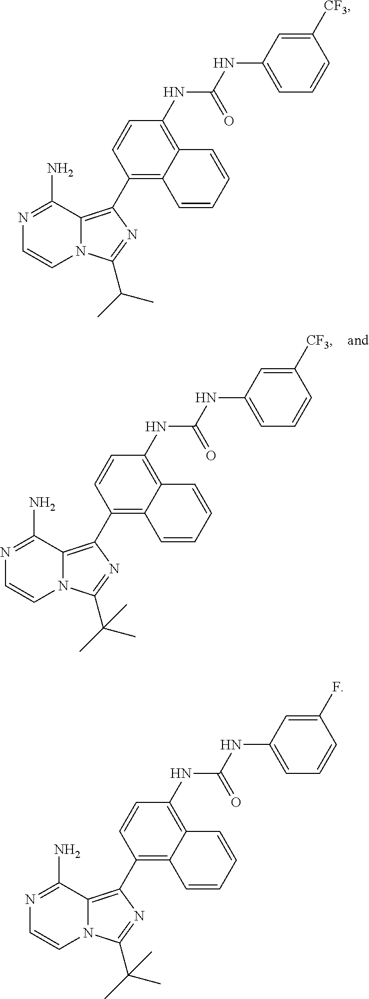 [SMILES: CC(C)c1nc(-c2ccc(NC(=O)Nc3cccc(C(F)(F)F)c3)c3ccccc23)c2c(N)nccn12 ; CC(C)(C)c1nc(-c2ccc(NC(=O)Nc3cccc(C(F)(F)F)c3)c3ccccc23)c2c(N)nccn12 ; "and" ; CC(C)(C)c1nc(-c2ccc(NC(=O)Nc3cccc(F)c3)c3ccccc23)c2c(N)nccn12]

41. (canceled)

42. (canceled)

43. The pharmaceutical composition of claim 38, wherein said Ire1 ribonuclease modulating compound is STF-083010, MKC-3946, 4μ8C, 3-methoxy-6-bromosalicylaldehyde, a salicylaldehyde, or toyocamycin.

44. (canceled)

45. (canceled)


SEQUENCE LISTING (26 sequences)

SEQ ID NO: 1 - protein, 538 aa, Homo sapiens
MSYYHHHHHHDYDIPTTENLYFQGAMDPEHQQQQLQHQQFQKELEKIQLLQQQQQQLPFHPPGDTAQDGELLDTSGPYSESSGTSSPSTSPRASNHSLCSGSSASKAGSSPSLEQDDGDEETSVVIVGKISFCPKDVLGHGAEGTIVYRGMFDNRDVAVKRILPECFSFADREVQLLRESDEHPNVIRYFCTEKDRQFQYIAIELCAATLQEYVEQKDFAHLGLEPITLLQQTTSGLAHLHSLNIVHRDLKPHNILISMPNAHGKIKAMISDFGLCKKLAVGRHSFSRRSGVPGTEGWIAPEMLSEDCKENPTYTVDIFSAGCVFYYVISEGSHPFGKSLQRQANILLGACSLDCLHPEKHEDVIARELIEKMIAMDPQKRPSAKHVLKHPFFWSLEKQLQFFQDVSDRIEKESLDGPIVKQLERGGRAVVKMDWRENITVPLQTDLRKFRTYKGGSVRDLLRAMRNKKHHYRELPAEVRETLGSLPDDFVCYFTSRFPHLLAHTYRAMELCSHERLFQPYYFHEPPEPQPPVTPDAL

SEQ ID NO: 2 - protein, 509 aa, Homo sapiens
HQQQQLQHQQFQKELEKIQLLQQQQQQLPFHPPGDTAQDGELLDTSGPYSESSGTSSPSTSPRASNHSLCSGSSASKAGSSPSLEQDDGDEETSVVIVGKISFCPKDVLGHGAEGTIVYRGMFDNRDVAVKRILPECFSFADREVQLLRESDEHPNVIRYFCTEKDRQFQYIAIELCAATLQEYVEQKDFAHLGLEPITLLQQTTSGLAHLHSLNIVHRDLKPHNILISMPNAHGKIKAMISDFGLCKKLAVGRHSFSRRSGVPGTEGWIAPEMLSEDCKENPTYTVDIFSAGCVFYYVISEGSHPFGKSLQRQANILLGACSLDCLHPEKHEDVIARELIEKMIAMDPQKRPSAKHVLKHPFFWSLEKQLQFFQDVSDRIEKESLDGPIVKQLERGGRAVVKMDWRENITVPLQTDLRKFRTYKGGSVRDLLRAMRNKKHHYRELPAEVRETLGSLPDDFVCYFTSRFPHLLAHTYRAMELCSHERLFQPYYFHEPPEPQPPVTPDAL

SEQ ID NO: 3 - protein, 292 aa, Homo sapiens
MGHHHHHHSSGVDLGTENLYFQSMDKWEMERTDITMKHKLGGGQYGEVYVGVWKKYSLTVAVKTLKEDTMEVEEFLKEAAVMKEIKHPNLVQLLGVCTLEPPFYIVTEYMPYGNLLDYLRECNREEVTAVVLLYMATQISSAMEYLEKKNFIHRDLAARNCLVGENHVVKVADFGLSRLMTGDTYTAHAGAKFPIKWTAPESLAYNTFSIKSDVWAFGVLLWEIATYGMSPYPGIDLSQVYDLLEKGYRMEQPEGCPPKVYELMRACWKWSPADRPSFAETHQAFETMFHDS

SEQ ID NO: 4 - protein, 286 aa, Gallus gallus
GHMQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGEMGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKDPEERPTFEYLQAFLEDYFTSTEPQYQPGENL

SEQ ID NO: 5 - protein, 432 aa, Homo sapiens
MGSSPSLEQDDGDEETSVVIVGKISFCPKDVLGHGAEGTIVYRGMFDNRDVAVKRILPECFSFADREVQLLRESDEHPNVIRYFCTEKDRQFQYIAIELCAATLQEYVEQKDFAHLGLEPITLLQQTTSGLAHLHSLNIVHRDLKPHNILISMPNAHGKIKAMISDFGLCKKLAVGRHSFSRRSGVPGTEGWIAPEMLSEDCKENPTYTVDIFSAGCVFYYVISEGSHPFGKSLQRQANILLGACSLDCLHPEKHEDVIARELIEKMIAMDPQKRPSAKHVLKHPFFWSLEKQLQFFQDVSDRIEKESLDGPIVKQLERGGRAVVKMDWRENITVPLQTDLRKFRTYKGGSVRDLLRAMRNKKHHYRELPAEVRETLGTLPDDFVCYFTSRFPHLLAHTYRAMELCSHERLFQPYYFHEPPEPQPPVTPDAL

SEQ ID NO: 6 - DNA, 20 bp, Artificial sequence
  Synthetic polynucleotide
ctgatggaggcacagtgaga

SEQ ID NO: 7 - DNA, 20 bp, Artificial sequence
  Synthetic polynucleotide
ctcgggtggagtgcttagag

SEQ ID NO: 8 - DNA, 20 bp, Artificial sequence
  Synthetic polynucleotide
agttcaacggcacagtcaag

SEQ ID NO: 9 - DNA, 19 bp, Artificial sequence
  Synthetic polynucleotide
actcagcaccagcatcacc

SEQ ID NO: 10 - DNA, 18 bp, Artificial sequence
  Synthetic polynucleotide
gtcctctgggagcccaag

SEQ ID NO: 11 - DNA, 17 bp, Artificial sequence
  Synthetic polynucleotide
acagagcctccaccagg

SEQ ID NO: 12 - DNA, 20 bp, Artificial sequence
  Synthetic polynucleotide
tgaaccgctgtcttgagatg

SEQ ID NO: 13 - DNA, 20 bp, Artificial sequence
  Synthetic polynucleotide
tcttggttgcctcttttgct

SEQ ID NO: 14 - DNA, 20 bp, Artificial sequence
  Synthetic polynucleotide
caaggagctgcaggagaaga

SEQ ID NO: 15 - DNA, 20 bp, Artificial sequence
  Synthetic polynucleotide
gcctggttgaagttctccac

SEQ ID NO: 16 - DNA, 20 bp, Artificial sequence
  Synthetic polynucleotide
gcaagtgcttctaggcggac

SEQ ID NO: 17 - DNA, 22 bp, Artificial sequence
  Synthetic polynucleotide
aagaaagggtgtaaaacgcagc

SEQ ID NO: 18 - DNA, 21 bp, Artificial sequence
  Synthetic polynucleotide
aaacagagtagcacagactgc

SEQ ID NO: 19 - DNA, 26 bp, Artificial sequence
  Synthetic polynucleotide
ggatctctaagactagaggcttggtg

SEQ ID NO: 20 - DNA, 26 bp, Artificial sequence
  Synthetic polynucleotide
aggaaactgaaaaacagagtagcagc

SEQ ID NO: 21 - DNA, 21 bp, Artificial sequence
  Synthetic polynucleotide
tccttctgggtagacctctgg

SEQ ID NO: 22 - RNA, 19 nt, Artificial sequence
  Synthetic polynucleotide
cugaguccgcagcacucag

SEQ ID NO: 23 - DNA, 45 bp, Artificial sequence
  Synthetic polynucleotide
gaaattaatacgactcactatagggggtccaggtacatccccgag

SEQ ID NO: 24 - DNA, 21 bp, Artificial sequence
  Synthetic polynucleotide
ttaggctggagccacctggct

SEQ ID NO: 25 - DNA, 4005 bp, Homo sapiens
tgcctagtcagttctgcgtccgctgaggctcggtcaccgcctcgctgtcgtcgcggcgcccccgccccgtcctctgtccgtaccgcccccggagccagggccgagtcctcgccatgccggcccggcggctgctgctgctgctgacgctgctgctgcccggcctcgggatttttggaagtaccagcacagtgacgcttcctgaaaccttgttgtttgtgtcaacgctggatggaagtttgcatgctgtcagcaagaggacaggctcaatcaaatggactttaaaagaagatccagtcctgcaggtcccaacacatgtggaagagcctgcctttctcccagatcctaatgatggcagcctgtatacgcttggaagcaagaataatgaaggcctgacgaaacttccttttaccatcccagaattggtgcaggcatccccatgccgaagttcagatggaatcctctacatgggtaaaaagcaggacatctggtatgttattgacctcctgaccggagagaagcagcagactttgtcatcggcctttgcagatagtctctgcccatcaacctctcttctgtatcttgggcgaacagaatacaccatcaccatgtacgacaccaaaacccgagagctccggtggaatgccacctactttgactatgcggcctcactgcctgaggacgacgtggactacaagatgtcccactttgtgtccaatggtgatgggctggtggtgactgtggacagtgaatctggggacgtcctgtggatccaaaactacgcctcccctgtggtggccttttatgtctggcagcgggagggtctgaggaaggtgatgcacatcaatgtcgctgtggagaccctgcgctatctgaccttcatgtctggggaggtggggcgcatcacaaagtggaagtacccgttccccaaggagacagaggccaagagcaagctgacgcccactctgtatgttgggaaatactctaccagcctctatgcctctccctcaatggtacacgagggggttgctgtcgtgccccgcggcagcacacttcctttgctggaagggccccagactgatggcgtcaccattggggacaagggggagtgtgtgatcacgcccagcacggacgtcaagtttgatcccggactcaaaagcaagaacaagctcaactacttgaggaattactggcttctgataggacaccatgaaaccccactgtctgcgtctaccaagatgctggagagatttcccaacaatctacccaaacatcgggaaaatgtgattcctgctgattcagagaaaaagagctttgaggaagttatcaacctggttgaccagacttcagaaaacgcacctaccaccgtgtctcgggatgtggaggagaagcccgcccatgcccctgcccggcccgaggcccccgtggactccatgcttaaggacatggctaccatcatcctgagcaccttcctgctgattggctgggtggccttcatcatcacctatcccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctgagcgagggcggcccctctgttctggtggccccagctgtgactgagggcctggtcaccacaattagagcttgatgcctcccggctttgcagggagaccaggcttcccaaaccaagtgccttgagctgcctgctctgcagcccacagaggacagtgctgaccccaggaagtgggagaagtggcccctcgtgacctacagggaactgggaagatgctggccccaaaagccttacggtcatgatgtctgcaaaggagggcctcagagacagcgcgagtagcacccccagccatctactggataaacttgcttcagactttttaaattcctgcttaatgtcagtctacaggcctttcaggaagggagaggagggaatcgtacattttgcttgcgtgctgggacagctaggctgagatgcaccaagtacagccttcactggagaccggaattgagaggtgggggatgctgaggagggggaggacggagttcagagggtgtcgtcctgcagtgtgagatttctcattgatcacagatgtgcccagagtagcccaggtcactgttaactagtgtttctgcagaggcagcaggagccatgagcatgaggtgtggcattagggactggtcagctatgcatgctggcaggtggggttgtgtctgcaggtctcagaaatgaagaggctgctctgttctggaggcagccgtggcccagtgccagtggccagaacagtggcctttggtgggtgtgtcccgggccatctcggggtggtgctcaggagcgcctggggcaagaggtaaagagttccctggccttcaaggagagcagcgaagacccagacaggggccagccttcaggaccagagggaggccgccgaatgggaccctcctggtcaccaggagaaagccctgggccagcgagtaggcagtcaaactccttcgtccccaaggccggtggaacaagaggct

SEQ ID NO: 26 - protein, 977 aa, Homo sapiens
MPARRLLLLLTLLLPGLGIFGSTSTVTLPETLLFVSTLDGSLHAVSKRTGSIKWTLKEDPVLQVPTHVEEPAFLPDPNDGSLYTLGSKNNEGLTKLPFTIPELVQASPCRSSDGILYMGKKQDIWYVIDLLTGEKQQTLSSAFADSLCPSTSLLYLGRTEYTITMYDTKTRELRWNATYFDYAASLPEDDVDYKMSHFVSNGDGLVVTVDSESGDVLWIQNYASPVVAFYVWQREGLRKVMHINVAVETLRYLTFMSGEVGRITKWKYPFPKETEAKSKLTPTLYVGKYSTSLYASPSMVHEGVAVVPRGSTLPLLEGPQTDGVTIGDKGECVITPSTDVKFDPGLKSKNKLNYLRNYWLLIGHHETPLSASTKMLERFPNNLPKHRENVIPADSEKKSFEEVINLVDQTSENAPTTVSRDVEEKPAHAPARPEAPVDSMLKDMATIILSTFLLIGWVAFIITYPLSMHQQQQLQHQQFQKELEKIQLLQQQQQQLPFHPPGDTAQDGELLDTSGPYSESSGTSSPSTSPRASNHSLCSGSSASKAGSSPSLEQDDGDEETSVVIVGKISFCPKDVLGHGAEGTIVYRGMFDNRDVAVKRILPECFSFADREVQLLRESDEHPNVIRYFCTEKDRQFQYIAIELCAATLQEYVEQKDFAHLGLEPITLLQQTTSGLAHLHSLNIVHRDLKPHNILISMPNAHGKIKAMISDFGLCKKLAVGRHSFSRRSGVPGTEGWIAPEMLSEDCKENPTYTVDIFSAGCVFYYVISEGSHPFGKSLQRQANILLGACSLDCLHPEKHEDVIARELIEKMIAMDPQKRPSAKHVLKHPFFWSLEKQLQFFQDVSDRIEKESLDGPIVKQLERGGRAVVKMDWRENITVPLQTDLRKFRTYKGGSVRDLLRAMRNKKHHYRELPAEVRETLGSLPDDFVCYFTSRFPHLLAHTYRAMELCSHERLFQPYYFHEPPEPQPPVTPDAL